(19) United States
(12) Patent Application Publication
(10) Pub. No.: US 2023/0399302 A1
(43) Pub. Date: Dec. 14, 2023

(54) PROCESS FOR THE PREPARATION OF HETEROARYL-SUBSTITUTED SULFUR(VI) COMPOUNDS

(71) Applicant: H. LEE MOFFITT CANCER CENTER AND RESEARCH INSTITUTE, INC., Tampa, FL (US)

(72) Inventors: Justin M. Lopchuk, Tampa, FL (US); Zachary P. SHULTZ, Lakeland, FL (US); Thomas SCATTOLIN, San Diego, CA (US)

(21) Appl. No.: 18/034,439

(22) PCT Filed: Oct. 29, 2021
(86) PCT No.: PCT/US2021/057262

§ 371 Date: Apr. 28, 2023

Related U.S. Application Data
(60) Provisional application No. 63/107,016, filed on Oct. 29, 2020.

(51) Int. Cl. C07D 239/38; C07D 405/04; C07D 403/04; C07D 405/12; C07D 401/12; C07D 417/06; C07D 409/04; C07D 411/04; C07D 417/04; C07D 239/30; C07D 239/42; C07D 471/04; C07D 241/12; C07D 213/34; C07D 213/803; C07D 213/80; C07D 213/61; C07D 215/12; C07D 487/04; C07D 473/00; C07D 495/04; C07D 277/64

(52) U.S. Cl. CPC C07D 239/38; C07D 405/04; C07D 403/04; C07D 405/12; C07D 401/12; C07D 417/06; C07D 409/04; C07D 411/04; C07D 417/04; C07D 239/30; C07D 239/42; C07D 471/04; C07D 241/12; C07D 213/34; C07D 213/803; C07D 213/80; C07D 213/61; C07D 215/12; C07D 487/04; C07D 473/00; C07D 495/04; C07D 277/64

(57) ABSTRACT

The present disclosure provides processes for the synthesis of organic compounds, in particular processes for the synthesis of heteroaryl-substituted sulfur(VI) compounds by nucleophilic aromatic substitution.

### CROSS-REFERENCE TO RELATED APPLICATIONS

This application claims the benefit of priority to U.S. Provisional Application No. 63/107,016, filed Oct. 29, 2020, the disclosure of which is incorporated herein by reference in its entirety.

### STATEMENT REGARDING FEDERALLY SPONSORED RESEARCH OR DEVELOPMENT

This invention was made with government support under Grant No. R35GM142577 awarded by the National Institutes of Health. The Government has certain rights in the invention.

### TECHNICAL FIELD

This disclosure relates to processes for the synthesis of organic compounds, and more particularly to methods for the synthesis of heteroaryl-substituted sulfur(VI) compounds.

### BACKGROUND

The ability of sulfur to adopt a range of oxidation states (II-VI) with defined molecular geometries has led to many advancements in the discovery sciences.¹⁻⁴ From materials to medicines, sulfur-containing functional groups are pervasive across disciplines. The more common S(VI) functional groups, such as sulfones and sulfonamides, have attracted the most attention—finding their way into many drug discovery programs and greater than 70 FDA approved drugs.⁵ More recently, the exploration of historically neglected sulfonimidoyl S(VI) functional groups, containing S═N and S—N bond(s), has provided novel clinical candidates for a variety of indications as well as important agrochemicals (FIG. 1A).⁶⁻¹²

Sulfoximines, the mono-aza S═N variants of sulfones (found in 1-5),¹³⁻¹⁸ have recently been accepted in medicinal chemistry as bioisosteres or viable replacement groups for carboxylic acids, alcohols sulfones and sulfonamides (FIG. 1B).^{6,19} Additionally, sulfonimidamides can serve as bioisosteres for amines, sulfones and sulfonamides.^{9,12} The unique H-bond donor and acceptor properties of the sulfonimidoyl groups allow them to mimic a wide range of other functionality while commonly providing other advantages, such as a chiral environment and increases in aqueous solubility.^{9,20} Recent enthusiasm over the physiochemical properties of sulfoximines (and other sul-fonimidoyl groups)^{8,9,19} has led to an exponential increase in their use to improve pharmacokinetic (PK) and pharmacodynamic (PD) properties during lead optimization studies.^{6-8,21}

A specific example of this was demonstrated by AstraZeneca during the discovery and subsequent development of their ATR inhibitor, ceralasertib (1).¹³ Amid the final optimization stage of the drug discovery program, a sulfone was replaced for a sulfoximine. The resulting introduction of a sulfoximine led to an increased aqueous solubility while maintaining potency, which allowed for the advancement of 1 to the clinic where it is currently undergoing multiple phase II clinical trials.²² Other discovery programs at Bayer,^{13,14,18,23} Pfizer,²⁴ Genentech,^{25,26} Hoffman-La Roche,²⁷ Novartis,²⁸ Nestle Skin Health²⁹ and Corteva Agriscience,³⁰ have been actively researching neglected S(VI) functional groups with respect to methods for their installation and incorporation into lead scaffolds.

Furthermore, the novelty of sulfonimidoyl groups, with their inherent stereochemical and additional spatial vectors capable of modifications, provides ample opportunities for new intellectual property (IP) development. An increase in patent applications and issuances within the last ten years is a growing testament to the untapped potential of sulfoximines (2,074 total patents since their first report in 1953, 1,536 of those coming in the last decade)³¹, sulfonimidamides (140 total patents since their first report in 1967, 121 of those coming in the last decade)³², sulfondiimines (33 total patents since their first report in 1967, 18 of those coming in the last decade)³³ and related functional groups. Pioneering work by Bolm,³⁴⁻³⁶ Bull,³⁷⁻³⁹ Johnson,⁴⁰ Luecking,⁴¹⁻⁴³ Maruoka,^{44,45} Sharpless,⁴⁶⁻⁴⁸ Willis⁴⁹⁻⁵¹ and others for the creation and modifications of S(VI) functionality has given rise to new possibilities in the field.⁵²⁻⁵⁴ However, despite the increase in methods to access neglected sulfonimidoyl-containing compounds, there has been relatively little advancement toward the modular installation of these groups to pharmaceutical scaffolds.

Owing to the growing attention of higher order sulfur-based functional groups as bioisosteres and PK modulators in the pharmaceutical sciences, the unmet need for their incorporation into medicinally relevant structures with an emphasis on asymmetric control must be addressed. Traditional methods to introduce α-substituted sulfonimidoyl units relies on S—C bond disconnection (a) (FIG. 1C) involving a laborious 4-6 step synthetic sequence from carboxylic acids or esters.^{12-14,16-18} Limitations of disconnection (a) include the lengthy step count, challenging asymmetric control at the stereogenic S-center and the difficulty of late-stage modifications at sulfur—the last of which explains the high prevalence of methyl substituted sulfoximines and sulfondiimines. Other methods for the synthesis of α-arylated sulfoximines rely on exotic transition metal-catalyzed systems and conditions accompanied by limited scope with respect to both sulfoximines and aryl coupling partners—especially with regards to heterocycles.^{41,55} There is a clear need for new methods for the synthesis of heteroaryl substituted sulfur(VI) compounds. The present disclosure addresses these as well as other needs.

### SUMMARY

In accordance with the purposes of the disclosed materials and methods, as embodied and broadly defined herein, the disclosed subject matter, in one aspect, related to processes for the synthesis of compounds as well as compounds made by said processes.

Thus, in one aspect, a process is provided for the preparation of a compound of Formula I

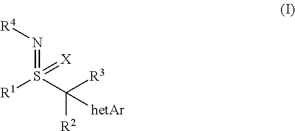

comprising reacting a compound of Formula II

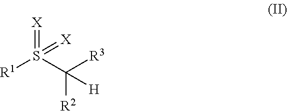

with a compound of Formula III

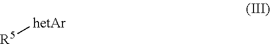

in the presence of a base;

wherein all variables are as defined herein.

Further provided are compounds of Formula I prepared by the processes described further herein.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF DRAWINGS

FIG. 1A shows biologically active heterocycles containing α-substituted sulfonimidoyl functional groups.

FIG. 1B shows the structural and physiochemical features of sulfonimidoyls.

FIG. 1C shows different disconnections to install sulfonimidoyl functional groups.

FIG. 2A shows synthetic routes to access S(VI) nucleophiles.

FIG. 2B shows optimization of sulfonimidoyl S_{N}Ar conditions with heterocycles. All reactions were performed on 0.25 mmol scale. ^{a}Isolated yields. ^{b}4,6-Dichloro-2-(methylthio)pyrimidine was used. ^{c}2-Fluoropyridine was used. ^{d}Bz transfer to α-position of sulfoximine observed. THF was used with NaHMDS. Dioxane was used with NaH and 15-crown-5 ether.

FIG. 3A shows synthetic route analysis of BAY 1251152.

FIG. 3B shows synthesis of BAY 1251152 via sulfonimidoyl SNAr approach.

FIG. 4A shows synthetic route analysis of antibacterial 4 and CDK9 inhibitor 2.

FIG. 4B shows the synthesis of pyridyl sulfoximine units via sulfonimidoyl S_{N}Ar.

FIG. 5A shows various routes towards ceralasertib (1) by AstraZeneca along with two sulfonimidoyl S_{N}Ar routes.

FIG. 5B shows target-oriented gram scale enantiospecific synthesis of 1.

FIG. 5C shows medicinal chemistry route to 1 via sulfonimidoyl S_{N}Ar outlining points of diversity.

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of”.

As used in the specification and the appended claims, the singular forms “a,” “an” and “the” include plural referents unless the context clearly dictates otherwise. Thus, for example, reference to “a compound”, “a base”, or “a solvent”, includes, but is not limited to, two or more such compounds, bases, solvents, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not.

### Chemical Definitions

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine.

Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙ-alkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one embodiment, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one embodiment, the alkanoyl group is optionally substituted as described herein.

“Haloalkoxy” indicates a haloalkyl group as defined herein attached through an oxygen bridge (oxygen of an alcohol radical).

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 3, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

“Protecting group”, as used herein, refers to any convenient functional group that allows to obtain chemoselectivity in a subsequent chemical reaction. Protecting groups are described, for example, in Greene & Wuts, eds., “Protecting Groups in Organic Synthesis”, 2nd ed. New York; John Wiley & Sons, Inc., 1991. For a particular compound and/or a particular chemical reaction, a person skilled in the art knows how to select and implement appropriate protecting groups and synthetic methods. Examples of amine protecting groups include acyl and alkoxycarbonyl groups, such as t-butoxycarbonyl (BOC), and [2-(trimethylsilyl)ethoxy]methyl (SEM). Examples of carboxyl protecting groups include (1-6C)alkyl groups, such as methyl, ethyl and t-butyl. Examples of alcohol protecting groups include benzyl, trityl, silyl ethers, and the like.

“Leaving group”, as used herein, refers to a molecule or a molecular fragment (e.g., an anion) that is displaced in a chemical reaction as stable species taking with it the bonding electrons. Examples of leaving groups include arylsulfonyloxy group or an alkylsulfonyloxy group, such as a mesylate or a tosylate group. Common anionic leaving groups also include halides such as Cl—, Br—, and I—.

As used herein, the term “composition” is intended to encompass a product comprising the specified ingredients in the specified amounts, as well as any product which results, directly or indirectly, from combination of the specified ingredients in the specified amounts.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

### Processes for the Preparation of Heteroaryl-Substituted Sulfur(VI) Derivatives

The present disclosure provides processes for the preparation of heteroaryl-substituted sulfur (VI) compounds, more particularly sulfoximines, sulfondiimines, sulfonimidamides, or derivatives thereof. More specifically, the present disclosure provides processes for the synthesis of heteroaryl-substituted sulfoxamine, sulfondiimine, or sulfonimidamide derivatives by a nucleophilic aromatic substitution reaction of an alkyl-substituted sulfoxamine, sulfondiimine, or sulfonimidamide with a heteroaryl compound substituted with a leaving group, for example a halo or sulfonate group, in the presence of a base.

Thus, in one aspect, a process is provided for the preparation of a compound of Formula I

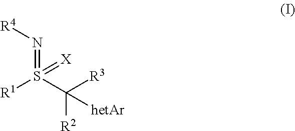

comprising reacting a compound of Formula II

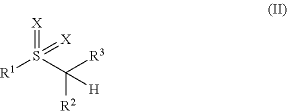

with a compound of Formula III

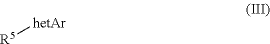

in the presence of a base;
wherein:
X is independently selected at each occurrence from O or NR⁴;
R¹ is selected from C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), (5- to 10-membered monocyclic or bicyclic heteroaryl)(C₀-C₃alkyl), or NR⁶R⁷, each of which may be optionally substituted as allowed by valence with one or more Y or Z groups;
R² and R³ are independently selected from hydrogen, C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), or (5- to 10-membered monocyclic or bicyclic heteroaryl)(C₀-C₃alkyl), each of which may be optionally substituted as allowed by valence with one or more Y or Z groups;
R⁴ is selected at each occurrence from hydrogen, C₁-C₆alkyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), (5- to 10-membered monocyclic or bicyclic heteroaryl)(C₀-C₃alkyl), C(═O)R⁸, or Si(R⁹)(R¹⁰)(R¹¹), each of which may be optionally substituted as allowed by valence with one or more Y or Z groups;
or R⁴ is a nitrogen protecting group;
or R¹ and R² may be brought together with the atoms to which they are attached to form a cycloalkyl or heterocycle ring, each of which may be optionally substituted as allowed by valence with one or more Y or Z groups;
or R² and R³ may be brought together with the carbon to which they are attached to form a cycloalkyl or heterocycle ring, each of which may be optionally substituted as allowed by valence with one or more Y or Z groups;
or R¹ and R⁴ may be brought together with the atoms to which they are attached to form a monocyclic or bicyclic heterocycle or heteroaryl ring, each of which may be optionally substituted as allowed by valence with one or more Y or Z groups;
R⁵ is a leaving group, for example a halo, a sulfonate, or a sulfone group;
hetAr is a monocyclic or bicyclic 6- to 10-membered heteroaryl group having one or more ring heteroatoms selected from N, O, and S, wherein at least one of the ring heteroatoms is N, and wherein hetAr may be optionally substituted as allowed by valence with one or more Y or Z groups;
R⁶ and R⁷ are each selected from hydrogen, C₁-C₆alkyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), and (5- to 10-membered monocyclic or bicyclic heteroaryl)(C₀-C₃alkyl), each of which may be optionally substituted with one or more Y or Z groups;
R⁸ is selected from hydrogen, C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), (5- to 10-membered monocyclic or bicyclic heteroaryl)(C₀-C₃alkyl), —OR^{a}, or —NR^{a}R^{b}, each of which may be optionally substituted as allowed by valence with one or more Y or Z groups;
R⁹, R¹⁰, and R¹¹ are independently selected from hydrogen, C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), and (5- to 10-membered monocyclic or bicyclic heteroaryl)(C₀-C₃alkyl), each of which may be optionally substituted as allowed by valence with one or more Y or Z groups;
Y is selected at each occurrence from halo, nitro, cyano, azido, oxo, C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), (5- to 10-membered monocyclic or bicyclic heteroaryl)(C₀-C₃alkyl), —OR^{a}, —SR^{a}, —NR^{a}R^{b}, —S(O)R^{c}, and —S(O)₂R^{c}, each of which may be optionally substituted with one or more Z groups as allowed by valence;
R^{a} and R^{b} are independently selected at each occurrence from hydrogen, C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), and (5- to 10-membered monocyclic or bicyclic heteroaryl)(C₀-C₃alkyl), each of which may be optionally substituted as allowed by valence with one or more Z groups;
R^{c} is independently selected at each occurrence from hydrogen, C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), (5- to 10-membered monocyclic or bicyclic heteroaryl)(C₀-C₃alkyl), —OR^{a}, —SR^{a}, and —NR^{a}R^{b}, each of which may be optionally substituted as allowed by valency with one or more Z groups; and
Z is independently selected at each occurrence from alkyl, haloalkyl, alkoxy, alkenyl, alkynyl, aryl, heteroaryl, aldehyde, amino, carboxylic ester, ether, halo, hydroxyl, ketone, cyano, silyl, sulfo-oxo, sulfonyl, sulfoxide, sulfonamide, thiol, or combinations thereof.

In alternative embodiments of the above process, hetAr may comprise a 5- to 10-membered aryl group substituted with at least one electron withdrawing group (for example, at least one halo group) and which may be further optionally substituted as allowed by valence with one or more Y or Z groups.

In some embodiments, the compound of Formula II is a compound of Formula II-a:

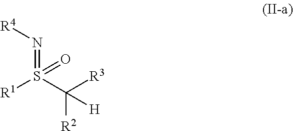

wherein all variables are as defined herein.

In some embodiments, the compound of Formula II is a compound of Formula II-b:

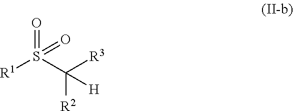

wherein all variables are as defined herein.

In some embodiments, the compound of Formula II is a compound of Formula II-c:

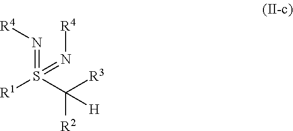

Wherein all variables are as defined herein.

In some embodiments, R¹ is C₁-C₆alkyl optionally substituted as allowed by valence with one or more Y or Z groups. In some embodiments, R¹ is NR⁶R⁷ optionally substituted as allowed by valence with one or more Y or Z groups. In some embodiments, R¹ is (6- to 10-membered monocyclic or bicyclic aryl)(C₀-C₃alkyl) optionally substituted as allowed by valence with one or more Y or Z groups. In some embodiments, R¹ is (5- to 10-membered monocyclic or bicyclic heteroaryl)(C₀-C₃alkyl) optionally substituted as allowed by valence with one or more Y or Z groups.

In some embodiments, R⁵ is a halo group, for example, fluoro, chloro, bromo, or iodo. In some embodiments, R⁵ is a sulfonate group, for example methanesulfonate, 4-methylphenylsulfonate, or trifluoromethylsulfonate. In some embodiments, R⁵ is a sulfone group, for example a methylsulfonyl group.

In some embodiments, hetAr is selected from the group consisting of pyridyl, pyrimidinyl, triazinyl, quinolinyl, or quinoxalinyl optionally substituted as allowed by valence with one or more Y or Z groups.

In alternative embodiments of the above process, hetAr may comprise a 5- to 10-membered aryl group substituted with at least one electron withdrawing group (for example, at least one halo group one haloalkyl group, one nitro group, one cyano group, or one carbonyl group) and which may be further optionally substituted as allowed by valence with one or more Y or Z groups.

In alternative embodiments of the above process, the compound of Formula II comprises a sulfone, for example but not limited to dimethyl sulfoxide of phenyl methyl sulfoxide.

In some embodiments, R⁴ is a nitrogen protecting group. Representative examples of nitrogen protecting groups include, but are not limited to, methoxymethyl, methylthiomethyl, p-methoxybenzyloxymethyl, p-nitrobenzyloxymethyl, t-butoxymethyl, 2-methoxyethoxymethyl, 1-ethoxyethyl, allyl, p-methoxybenzyloxycarbonyl (Moz), p-nitrobenzyloxycarbonyl (PNZ), trimethylsilyl, diethylisopropylsilyl, triphenylsilyl, formyl, chloroacetyl, methanesulfonyl, tosyl, benzylsulfonyl, methoxymethylcarbonyl, benzyloxycarbonyl, carboxybenzyl (Cbz), t-butyloxycarbonyl (BOC), 9-fluorenylmethylcarbonyl, N-phenylcarbamoyl, and 4,4′-dimethoxytrityl.

Representative examples of compounds of Formula II which may be used in the disclosed processes include, but are not limited to:

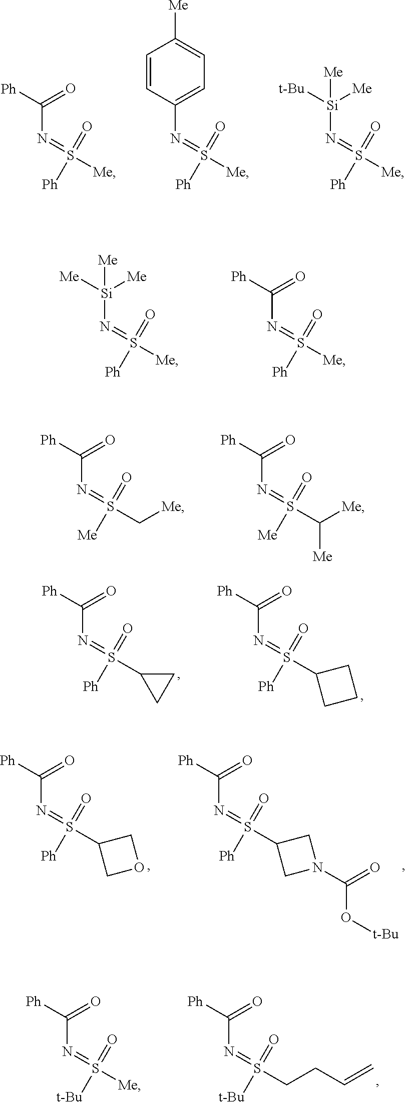

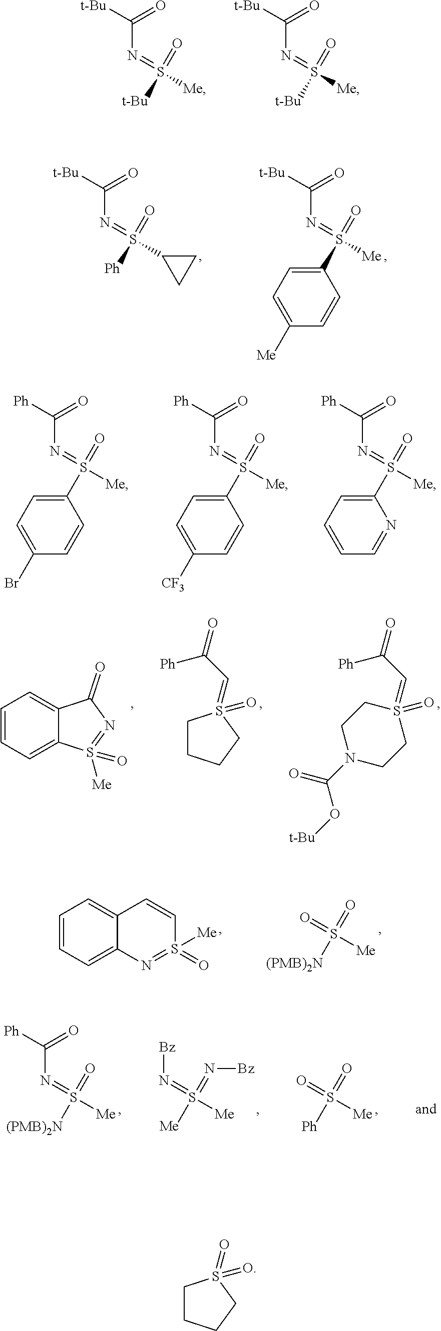

Further representative examples of compounds of Formula II which may be used in the disclosed processes include, but are not limited to:

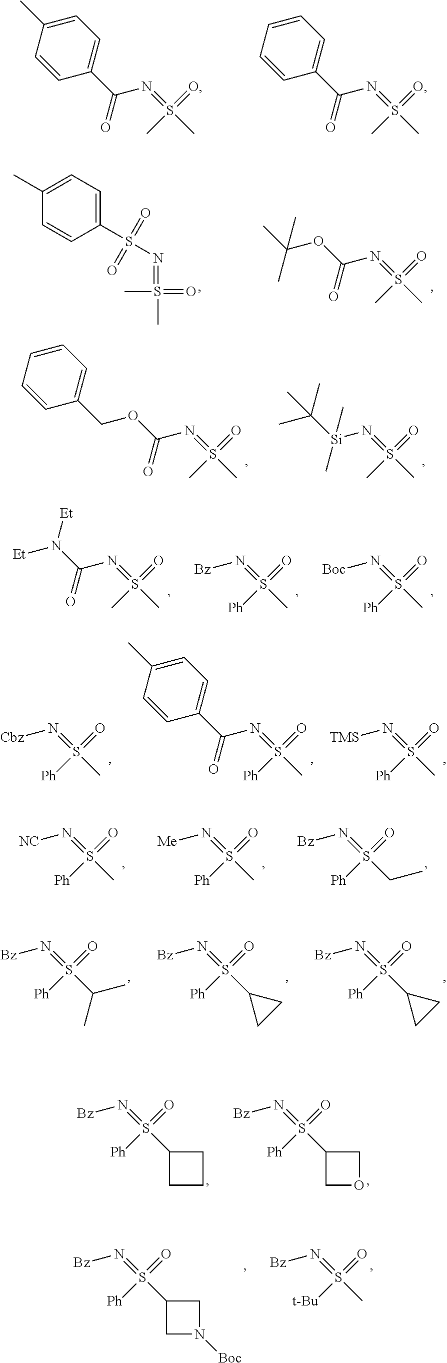

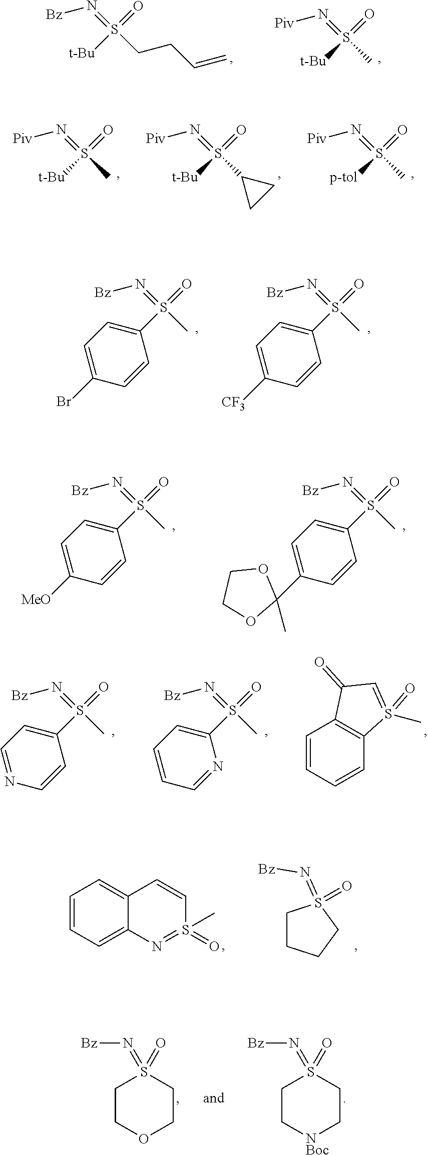

Representative examples of compounds of Formula III which may be used in the disclosed processes include, but are not limited to:

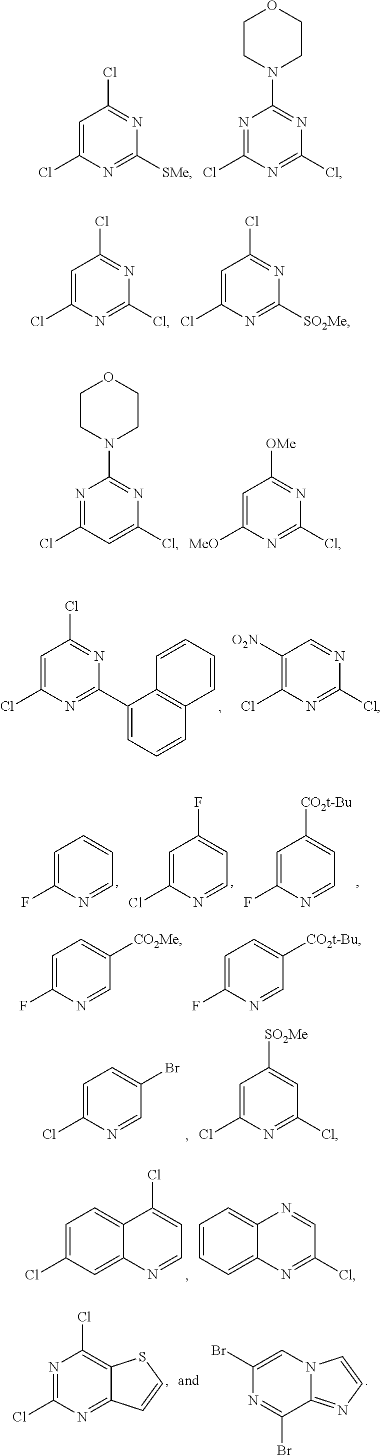

Further representative examples of compounds of Formula III which may be used in the disclosed processes include, but are not limited to:

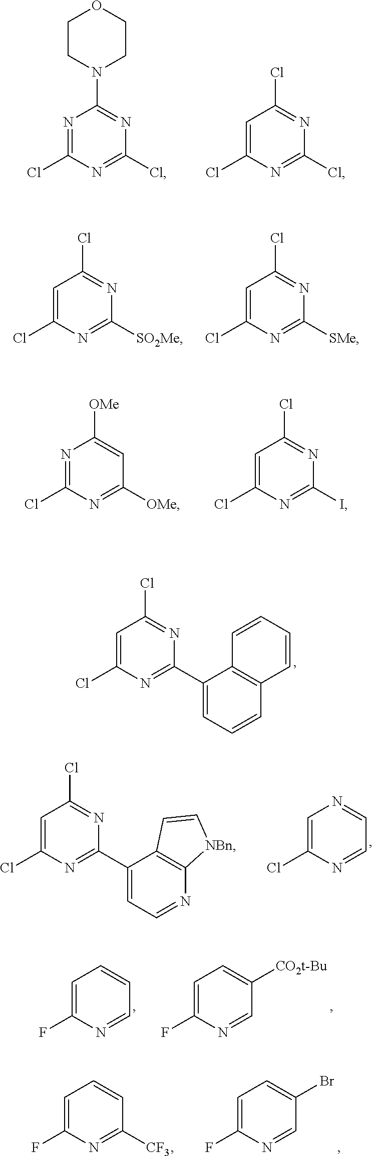

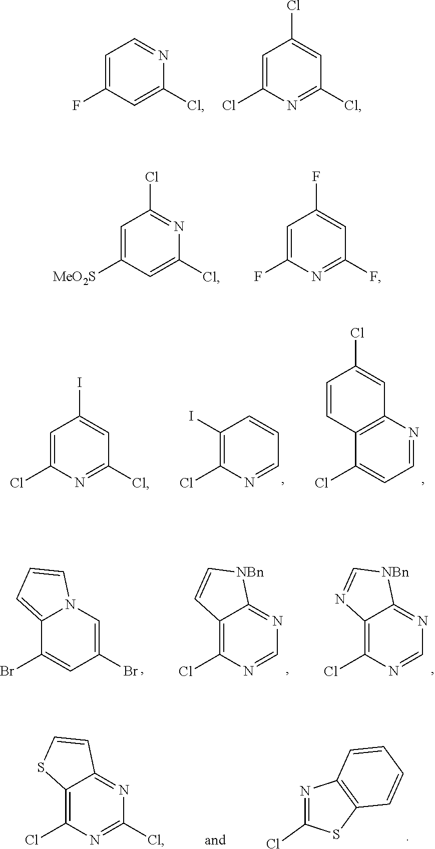

In alternative embodiments, representative examples of compounds of Formula III include, but are not limited to:

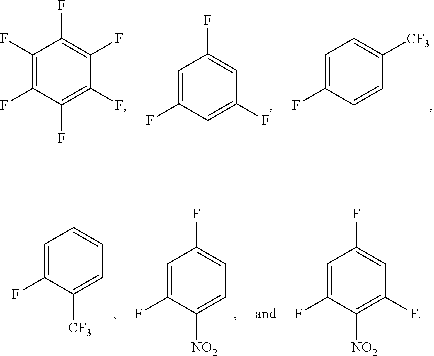

The base as used in the present process is typically a base having a sufficient pK_{b} to deprotonate the hydrogen atom pictured in Formula II as described herein. In some embodiments, the base may comprise an alkali or alkaline earth metal amide salt. In some embodiments, the base may comprise an alkali or alkaline earth metal salt of bis(trimethylsilyl)amide. In some embodiments, the base may comprise sodium bis(trimethylsilyl)amide, lithium bis(trimethylsilyl)amide, or potassium bis(trimethylsilyl)amide. In some embodiments, the base is sodium bis(trimethylsilyl)amide. Appropriate amide bases which may also be used may be readily identified by a person of ordinary skill in the art.

In other embodiments, the base may comprise a metal hydride salt, for example sodium hydride, potassium hydride, or cesium hydride. In such embodiments, a crown ether may be further included in the reaction mixture to help facilitate dissociation of the metal cation from the hydride base. Representative examples of crown ethers which may be used include 15-crown-5, 18-crown-6, and 21-crown-7.

In some embodiments, the reaction described herein may further be run in the presence of a solvent. The solvent may typically comprise one which is unreactive with the selection of base for use in the process. In typical embodiments, the solvent may comprise an ethereal solvent, for example tetrahydrofuran, diethyl ether, or 1,4-dioxane. Other suitable solvents may be used as would be apparent to a person of skill in the art.

In some embodiments, the base may comprise an alkali or alkaline earth metal salt and the solvent may comprise tetrahydrofuran. In some embodiments, the base may comprise sodium bis(trimethylsilyl)amide and the solvent may comprise tetrahydrofuran. In such embodiments, the process may be performed at a temperature ranging from about −78° C. to about 25° C.

In some embodiments, the base may comprise a metal hydride salt, the reaction mixture may further comprise a crown ether, and the solvent may comprise 1,4-dioxane. In some embodiments, the base may comprise sodium hydride, the reaction mixture may further comprise 15-crown-5, and the comprise 1,4-dioxane. In such embodiments, the process may be performed at a temperature ranging from about 25° C. to about 80° C.

Variations on compounds of Formula II or Formula III as used in the processes described herein can include the addition, subtraction, or movement of the various constituents as described for each compound. Similarly, when one or more chiral centers is present in a molecule the chirality of the molecule can be changed. Additionally, the synthesis of the compounds of Formula II or Formula III for use in the process can involve the protection of various chemical groups, and further the compounds of Formula I prepared by the disclosed may be subsequently deprotected as appropriate. The use of protection and deprotection, and the selection of appropriate protecting groups would be readily known to one skilled in the art. The chemistry of protecting groups can be found, for example, in Peter G. M. Wuts, Greene's Protective Groups in Organic Synthesis, 5^{th} Ed., Wiley & Sons, 2014.

The described processes, or reactions to produce the compounds used in the described processes, can be carried out in solvents indicated herein, or in solvents which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art.

For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

In another aspect, compounds of Formula III are also provided prepared by the processes described herein.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods the results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts temperature, etc.), but some errors and deviations should be accounted for. Unless indicated otherwise, parts are parts by weight, temperature is in degrees Celsius or is at ambient temperature, and pressure is at or near atmospheric. There are numerous variations and combinations of reaction conditions, e.g, component concentrations, temperatures, pressures, and other reaction ranges and conditions that can be used to optimize the product purity and yield obtained from the described process. Only reasonable and routine experimentation will be required to optimize such process conditions.

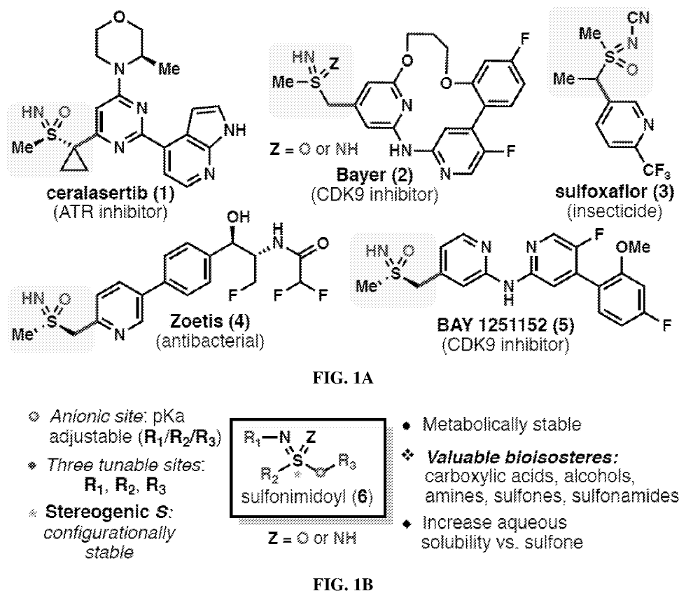

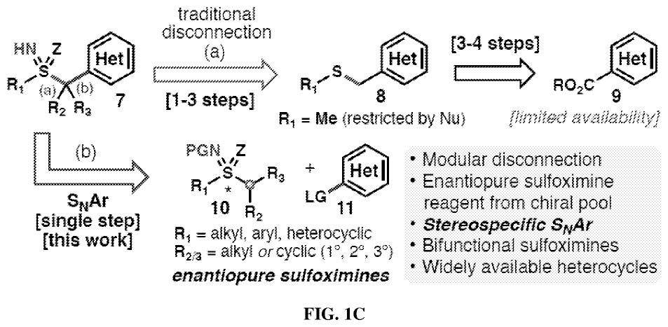

To address the aforementioned limitations and provide a modular approach to aid in discovery efforts, a straightforward solution was sought that can be applied to readily available pharmaceutically relevant heterocyclic building blocks. Disconnection (b) (FIG. 1C) outlines an S_{N}Ar approach for the installation of α-substituted sulfonimidoyl functionality to widely available electrophilic heterocycles. While scattered reports of sulfones undergoing S_{N}Ar exist,⁵⁶⁻⁵⁸ this alternative disconnection will provide discovery chemists with a multifunctional method for the introduction of highly oxidized neglected sulfur moieties of varying complexity in a single step. The utilization of enantiopure sulfonimidoyl nucleophiles grants an enantiospecific entry into α-heteroarylated products that will transform the targeted synthesis process, relieving the pressure of relying on chiral separation techniques and loss of material.

### Results and Discussion

### Development and Scope

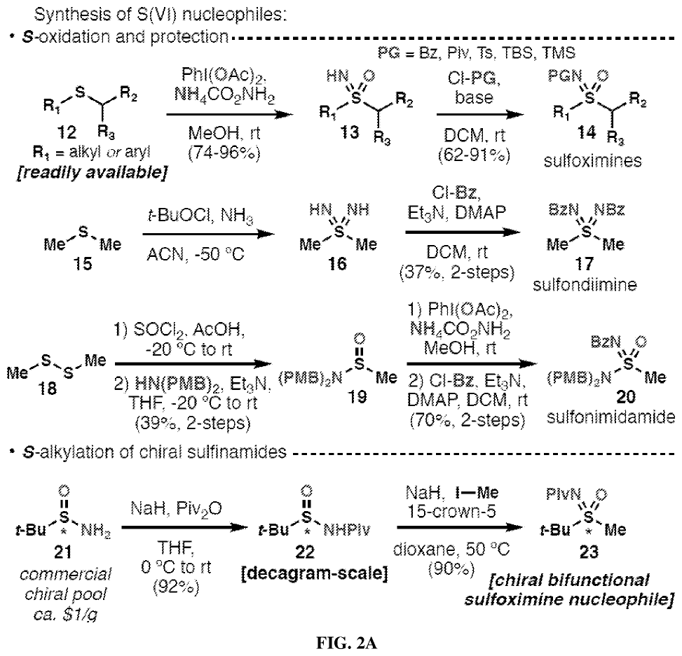

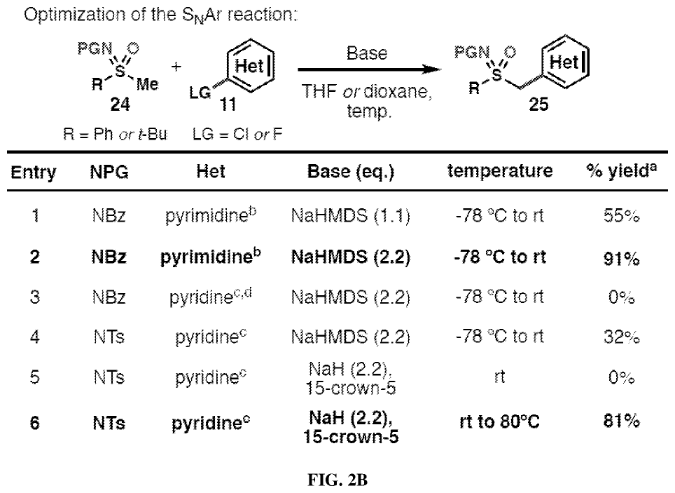

The requisite sulfoximines were accessible from the direct oxidation/imination of sulfides or sulfoxides via Bull and Luisi's sulfoximine synthesis,³⁹ followed by N—H protection (FIG. 2A). Although the modern method developed by Willis for the synthesis of sulfondiimines could be used to access dimethyl sulfondiimine^{16,50} the direct imination of dimethyl sulfide with t-BuOCl and NH₃ was chosen for this application.⁵⁹ Orthogonally protected sulfonimidamide 20 was made available from a 4 step procedure starting from disulfide 18. Chiral alkyl sulfoximines can be made readily available via Maruoka's S-alkylation strategy from N-Piv protected sulfinamide 22.⁴⁴ Enantiopure t-Bu methyl sulfoximine 23 provides a bench-stable chiral bifunctional sulfoximine linchpin, which will be highlighted in the synthetic applications below. To maintain the practical nature of an S_{N}Ar approach, we sought a general, operationally simple and scalable procedure that could be carried with minimal manipulations.

A variety of bases and reaction conditions were screened with respect to the S(VI) nucleophile 24 and electrophilic heterocycles 11 (FIG. 2B, entries 1-6). Two different procedures, cryogenic (NaHMDS, −78° C., entries 1-4) and thermal (NaH/15-crown-5, 50-80° C., entry 6), were found to be compatible with a wide range of electrophilic heterocycles. For heterocycles containing more than one electrophilic site, trace amounts of bis-S_{N}Ar product were observed and can be circumvented by adjusting addition rate of NaHMDS. Both methods were designed for ease of use by pre-mixing the nucleophile and electrophile followed by the addition of base. Due to the increased acidity of the α-H in the S_{N}Ar product 25 relative to the S(VI) nucleophile 24, at least 2 equivalents of base (entry 2) are required for 1° and 2° nucleophiles (1.1-1.5 equivalent for 3°) to obtain full conversions and high yields. Thermal conditions were employed for less electron-deficient heterocycles (entry 6), such as pyridines, that were unable to undergo the S_{N}Ar reaction at room temperature (heating the reaction mixtures with NaHMDS lead to decomposition and side product formation). In nearly all cases, a 1:1 stoichiometry of nucleophile and electrophile provides good to excellent yields under the optimized reaction conditions.

With two procedures in hand that provide reactivity with electron-deficient and (relatively) more electron-rich heterocycles, the S(VI) nucleophile scope was investigated. Symmetrical pyrimidine 26 was chosen to be the model heterocyclic electrophile due to its electronic nature as well as a practical scaffold for further synthetic manipulation. Various protecting groups including hydrolytically cleavable groups (28 N-COp-tol, 29 N-Bz,), commonly used tosyl (N-Ts, 30), N-Boc (31), N-Cbz (32) and silyl groups (N-TBS, 33; N-TMS, 39) were all compatible to provide good to excellent yields. When an N-TMS protecting group is employed, silyl group cleavage is observed under the work-up conditions to provide free sulfoximine 40 (N—H) in 77% yield, allowing for the introduction and deprotection of a sulfoximine unit to heterocycles in a single step. N-Cyano (N—CN, 41) sulfoximines, a commonly used imino N-substituent,⁶⁰ undergo the S_{N}Ar smoothly in high yields (86%).

A myriad of N-Bz protected sulfoximines were screened to provide a wide nucleophile scope as seen below. Sulfonimidoyl S_{N}Ar is not limited to the previously mentioned primary sulfoximines. Both secondary (43, 51) and tertiary dimethyl sulfoximine (44) examples resulted in the desired S_{N}Ar product in high yields. Cyclic α-substituted nucleophiles, such as cyclopropyl (45, 46), cyclobutyl (47) oxetane (48) and azetidine (49) provided sterically congested heterocyclic sulfoximines with increased molecular complexity in a single step. Enantiopure sulfoximines (52-55) were investigated and determined to undergo an enantiospecific S_{N}Ar reaction with pyrimidine 26 on gram-scale and in high yields (75-90%). The utility of this transformation is two-fold: 1) introduction of an asymmetric sulfoximine unit without erosion of enantiopurity (as determined by chiral HPLC) and 2) capability of further modifications at sulfur upon t-Bu cleavage, providing a platform for late-stage diversifications.

Arene-substituted sulfoximines containing electron donating and withdrawing groups were well tolerated (56-58). The presence of other acidic functionality (O—H or N—H) and enolizable groups (MeCO—R) were not compatible. Protection of these reactive groups, such as acetonide 59, provides access to masked carbonyl groups that can be later manipulated. Heterocycle-containing sulfoximines including pyridyl substituents (60, 61), a saccharin analog (62), and benzothiazine oxide (63) can be appended to other heterocyclic moieties in good yields (65-82%). In the cases of 62 and 63, method B was required due to the poor solubility of the nucleophiles at low temperatures. Cyclic aliphatic sulfoximine 64 and those containing heteroatoms (65, 66) were also compatible and gave diastereomeric mixtures (ca. 4:1 to 1:1) in good yields (62-78%).

With an established sulfoximine scope, we turned our attention to neglected sulfonimidamides and sulfondiimines to determine their compatibility under our optimized S_{N}Ar conditions. Orthogonally-protected sulfonimidamide 67 proved to be a suitable nucleophile that give the desired S_{N}Ar product in 82% yield—the first example of the direct installation of this functional group to a heterocycle. The bis N-Bz protected sulfondiimine 68 was also a suitable nucleophile using both method A (65% yield) and method B (92% yield). Classical oxidized sulfur groups, such as sulfonamides, sulfones and sulfoxides afforded the expected S_{N}Ar products in 77-99% yields (69-73). The six different gram-scale examples demonstrate the scalability of the method without a diminishment in yield.

Next, the electrophilic scope with regards to common place heterocyclic scaffolds in drug discovery was investigated. Three different sulfoximines were used to interrogate electrophilic reactivity, each with a different protecting group that proved critical. For most electron deficient systems (e.g. triazines and pyrimidines), a benzoyl (N-Bz) protecting group sufficed. For less reactive substrates, benzoyl transfer to the sulfoximine α-carbon was observed. To eliminate protecting group transfer, more robust PGs (N-Piv, N-Ts, N-Boc) were used. The use of both methods A and B allowed for an extensive electrophile scope that delivered a large variety of sulfoximine-containing heterocycles.

Electron-deficient ring systems known to readily undergo S_{N}Ar chemistry were first examined. Substituted 1,3,5-triazine (76) served as an excellent substrate along with pyrimidines that were substituted with electron donating groups (80, 81). When 2,4,6-trichloropyrimidine was used as an electrophile, a mixture of regioisomers (1:2, 77:78) was observed. Regioselective nu-cleophilic substitution on pyrimidine ring systems was achieved by a leaving group (LG) switch from chloro to SO₂Me to provide C-2 selective S_{N}Ar products 78 and 79 in high yields (93% and 89%). Trifunctional 4,6-dichloro-2-iodopyrimidine provided C-4 selective displacement of a chloro over iodo leaving group to give 82 and 83 as the major products on gram-scale. The utility of both S_{N}Ar products, 82 and 83, will be further demonstrated in the forthcoming synthetic applications. Other 2-substituted pyrimidines decorated with naphthyl (84) and azaindole (85) substituents resulted in the desired S_{N}Ar products in high yields (79-89%). The diazine scope is not limited to pyrimidines; 2-chloropyrazine also served as a suitable electrophile in good yield (86, 71%). Commercial and readily available pyridines were thoroughly explored. Initial attempts at affecting the sulfonimidoyl S_{N}Ar with 2-fluoropyridine under both cryogenic and thermal conditions using N-Bz protected sulfoximines proved unfruitful, due to Bz transfer to the sulfoximine starting material. To our delight, switching to more robust PGs (N-Ts, 87; N-Boc, 88) attenuates their reactivity allowing the S_{N}Ar to prevail.

An electron-deficient pyridine bearing a t-Bu ester (CO₂t-Bu) at the 3-position provided 89 in 72% yield. Trifluoromethyl-substituted pyridines proved troublesome under the optimized reaction conditions, reflective by a 22% isolated yield of 90. However, other halogenated pyridines underwent the S_{N}Ar smoothly to afford an array of highly useful pyridine products (91-95). Preferential displacement of fluoro over chloro was demonstrated with 2-chloro-4-fluoropyridine granting site-selective 4-substitution product 92. When 2,4,6-trichloropyridine was used, C-2 selectivity was observed in a modest, but still serviceable, 6.6:1 ratio of 93 (isolated r.r.). Site selectivity can be reversed by the replacement of chloro with —SO₂Me at the 4-position, where 94 was obtained in high yield (79%) as the sole regioisomer. It should be noted that when SO₂Me is used as a LG in the less reactive pyridine series, dimerization of the electrophile via S_{N}Ar with the sulfone is observed as a side-product (not observed with pyrimidines). Conversely, 2,4,6-trifluoropyridine was less selective for the 2-position (1.9:1 r.r.) to give 95 and 96 in 50% and 27% yields respectively. In the case where iodide could act as a LG, demonstrated by 2,6-dichloro-4-iodopyridine, a 19:1 regioselectivity was observed favoring substitution at the 2-position to provide 97. In addition, 2-chloro-3-iodopyridine was subjected to method B to give 3-iodo pyridyl sulfoximine 98 (44% yield) capable of further functionalization. The polyhalopyridine substrates examined provide unique opportunities for downstream modifications, via further S_{N}Ar and/or cross-coupling chemistry, which may serve as important intermediates for future discovery efforts.

Other medicinally relevant heterocyclic scaffolds, including 4,7-dichloroquinoline of the malaria drug chloroquine, were found to be suitable electrophiles for sulfonimidoyl S_{N}Ar. The first reported sulfoximine chloroquine analog 99 was made accessible in good yield (73%) and will be further investigated biologically. 6,8-Dibromoimidazo[1,2-a]pyrazine served as an excellent electrophile to afford 100 in high yield (84%). Appropriately halogenated and protected pyrrolo[2,3-d]pyrimidines, and purines were all compatible electrophiles under thermal sulfonimidoyl S_{N}Ar conditions to provide 101-103-enabling a route to novel sulfoximine nucleotide analogs. Sulfur containing heterocycles such as 2,4-dichlorothieno[2,3-d]pyrimidine, commonly found in PI3K inhibitors and non-nucleoside reverse transcriptase inhibitors,^{61,62} along with 2-chlorobenzothiazole proved to be suitable S_{N}Ar substrates that gave 104 and 105 in good yields respectively (71-84%).

Although this work focuses on providing a straightforward and robust method for the installation of methylene-linked sulfonimidoyl functional groups to pharmaceutically relevant heterocycles, a brief exploration of arene compatibility was warranted to understand the full scope of sulfonimidoyl S_{N}Ar chemistry. Hexafluorobenzene was reactive under cryogenic S_{N}Ar conditions (110, 89% yield) while 1,3,5-trifluorobenzene was not—more forceful thermal conditions were required (111, 77% yield). Interestingly, and in contrast to the pyridine example 90, 2- and 4-fluorotrifluorobenzene underwent an S_{N}Ar with method B to provide 112 and 113. To our surprise, and with modifications to the general procedure, fluoronitrobenzenes were able to serve as electrophiles to give sulfoximines 114 and 115. Based on a short arene screen, the scope of this transition metal-free method for the installation of S(VI) functionality is not limited to activated heterocycles but is also suitable for numerous electron deficient arene substrates.

### Synthetic Applications

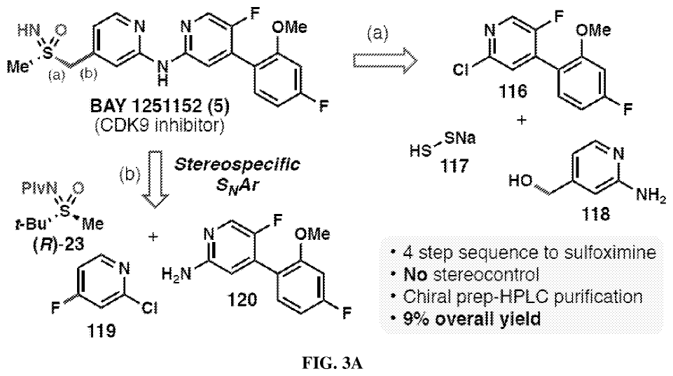

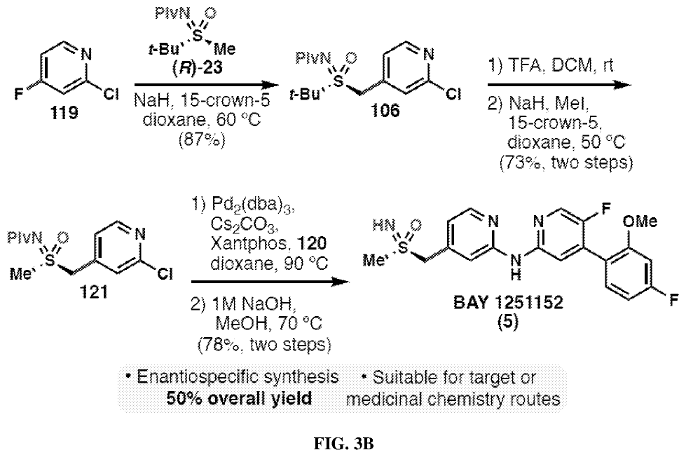

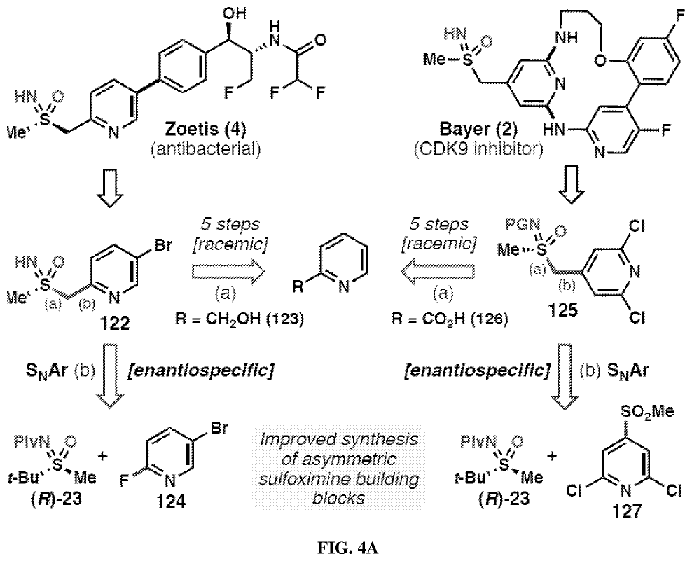

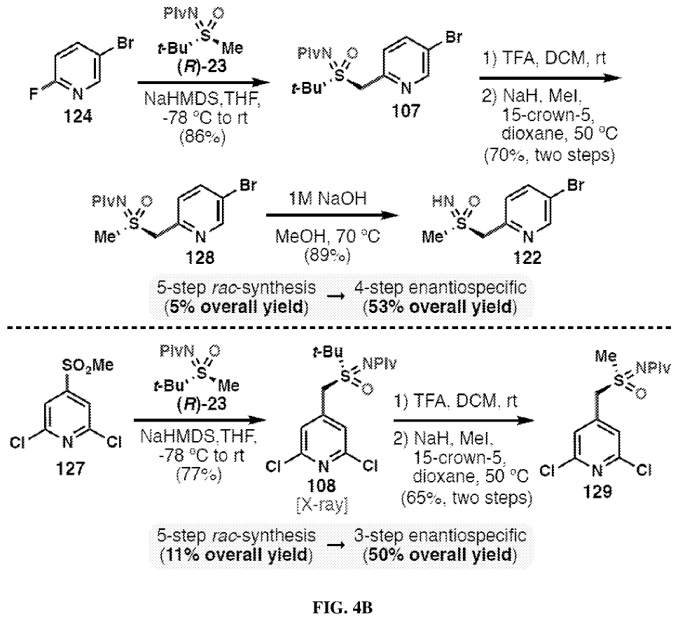

Current synthetic strategies to access α-substituted sulfoximines, and other neglected S(VI) groups, are typically arduous. Nucleophilic substitutions at activated benzylic positions by alkyl thiolates followed by oxidation to the desired sulfoximine are the most common routes, as demonstrated in the synthesis of BAY 1251152 (5, FIG. 3A, right).¹⁸ The recent disclosure of S-alkylations of sulfinamides with benzylic halides developed by Maruoka can circumvent the oxidation steps (1-2 steps), while providing enantiopure sulfoximine products.⁴⁴ In order to apply an S-alkylation strategy to access sulfoximines, the requisite benzylic halides (Br or I) are required via 2-3 step functionalizations of carboxylic acids or esters and is mainly limited to primary halides. An alternative S_{N}Ar strategy provides increased modularity and a large selection of electrophilic partners while maintaining enantiospecificity.

Bayer relied on the traditional approach, a thiolation/oxidation sequence (4 steps), to access the sulfoximine used in the synthesis of BAY 1251152. Their route resulted in a racemic mixture that was separated by preparative chiral HPLC in order to access the desired target. A more modular approach utilizing a stereospecific sulfoximine installation would expedite the target synthesis and aid in future analog discovery. Sulfonimidoyl S_{N}Ar was employed in the development of a concise synthesis of BAY 1251152 that is amenable for target and medicinal chemistry applications.

Beginning with commercially available 2-chloro-4-fluoro-pyridine (119) and our chiral bifunctional sulfoximine (R)-23, S_{N}Ar method B gave enantiomerically pure pyridyl sulfoximine 106 in high yield (87%, FIG. 3B). The two-step procedure for S-alkylation developed by Maruoka was employed to install the desired methyl (Me) sulfoximine. A previously reported method for the Buchwald-Hartwig coupling of 2-aminopyridine 120¹⁸ was adopted allowing coupling to 2-chloropyridyl sulfoximine 121. Deprotection of the N-Piv with NaOH furnished BAY 1251152 in 78% yield over two steps from 121. With the new sulfonimidoyl disconnection, the desired target was made accessible in 5 steps and 50% overall yield of the desired enantiomer from known starting materials—a more than five-fold increase in overall yield of BAY 1251152.

For the synthesis of Zoetis' pyridyl sulfoximine, commercially available 5-bromo-2-fluoropyridine (124) and chiral sulfoximine (R)-23 underwent a smooth S_{N}Ar using method A (107, 86% yield). The two-step S-functionalization sequence provided methyl pyridyl sulfoximine 128 in 70% yield. Deprotection of the pivaloyl group under basic hydrolysis conditions gave the desired free sulfoximine 122 in 53% overall yield as a single enantiomer. A reported Suzuki coupling of the requisite boronic ester with rac-122 provides access to 4.¹⁷ The improved route increased the overall yield of Zoetis' chiral sulfoximine intermediate from 5% to 53% overall yield and decreased the step count while providing a diversifiable intermediate for analog development (N-Piv sulfinamide after t-Bu cleavage).

Bayer's macrocyclic CDK9 inhibitor 2 was made accessible from (rac)-2,6-dichloropyridyl sulfoximine 125 where the protecting group was Cbz or Boc—introduced from the imination step.¹⁴ A pivaloyl protected 2,6-dichloropyridyl sulfoximine should serve the same purpose and is expected to be compatible with the subsequent chemistry for the synthesis of macrocycle 2. To prepare the appropriately protected sulfoximine, pyridyl sulfone 127 and chiral sulfoximine (R)-23 were subjected to S_{N}Ar method A giving enantiopure t-Bu sulfoximine 108. The two step S-alkylation sequence resulted in the desired methyl pyridyl sulfoximine intermediate 129 in 50% overall yield in 3 steps from readily available starting materials. The overall yield was improved from 11% to 50% while decreasing the step count by two. This expedient 3 step synthesis of the chiral sulfoximine building block for Bayer's macrocycle CDK9 inhibitor aids in targeted synthetic efforts while providing another platform for analog development.

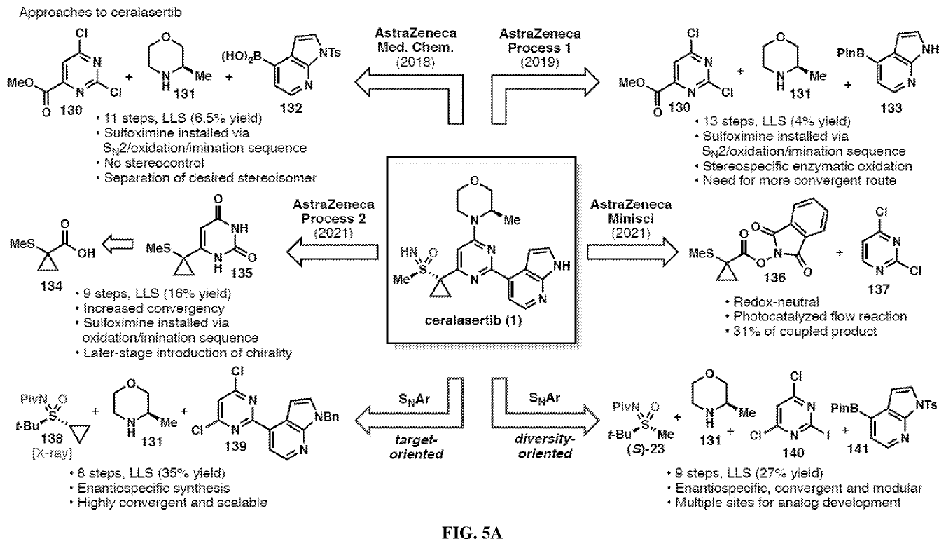

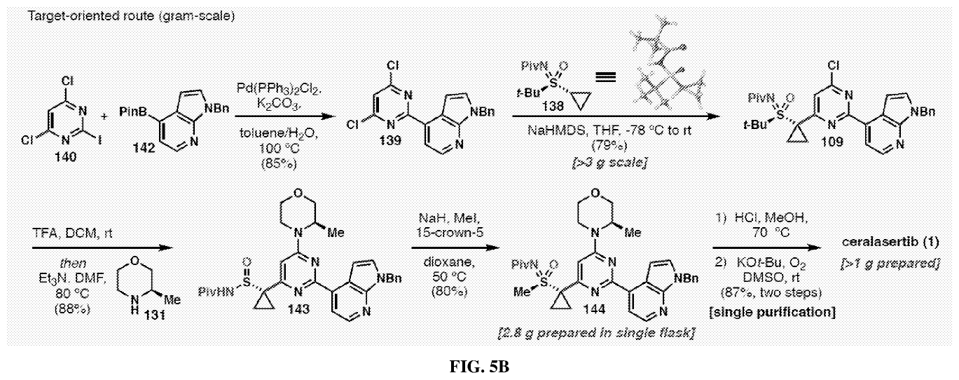

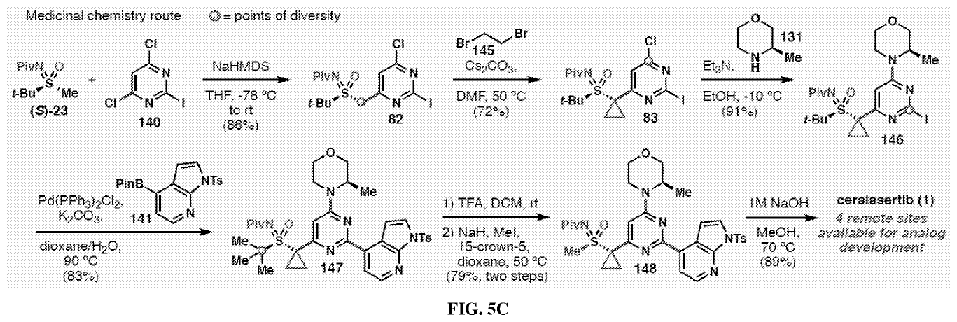

One of the most noteworthy developments pertaining to the use of sulfonimidoyl functional groups in medicinal chemistry is the development of the AstraZeneca's ATR inhibitor ceralasertib (1). In order to provide sufficient quantities of 1 for evaluation in clinical trials, the process chemistry team at Astra-Zeneca (AZ) had to revamp the medicinal chemistry route that was disclosed in 2018 (FIG. 5A, top left).¹³ Owing to the traditional method of α-substituted sulfoximine installation, the medicinal chemistry group produced 1 (and analogs) as mixtures of diastereomers that were separated by column chromatography or iterative recrystallizations. In order to overcome the cumbersome purifications and to increase overall yield of the desired diastereomer, AZ's process team subsequently (FIG. 5A, top right) utilized an enantioselective enzymatic oxidation to introduce the chirality at sulfur (as a sulfoxide) that was later imitated to give the desired sulfoximine functionality.⁶³ One of the major drawbacks with their 13 step process synthesis was the installation of the cyclopropyl ring system found in 1.

As the AZ process team described: “The manufacture of AZD6738 remains a challenge for the future of this medicine, due to the difficult nature of installing the dense functionality around the pyrimidine core. Longer term it would be beneficial to have a more convergent approach with a higher yielding route”.⁶³ During the development of our sulfonimidoyl S_{N}Ar method, AZ's process team disclosed an improved route (FIG. 5A, middle left)⁶⁴ and an attempt at a photocatalyzed flow approach via a Minisci reaction (FIG. 5A, middle right)⁶⁵. The newly developed process route to 1 (FIG. 5A, middle left) addressed the issue of installing a cyclopropyl methyl sulfide moiety from 134 and 135 in a single step, while still relying on an enantioselective enzymatic oxidation/imination sequence to access the sulfoximine. To address the remaining drawbacks of the AZ's process route, we developed an enantiospecific convergent approach utilizing our S_{N}Ar method to generate the core structure of ceralasertib (1) from enantiopure t-butyl cyclopropyl sulfoximine 138 and pyrimidine 139 (FIG. 5A, bottom left). Concurrently, an alternative medicinal chemistry-oriented synthesis of 1 was developed (FIG. 5A, bottom right) to provide multiple points of diversity that would aid in analog development for related scaffolds of 1.

The target-oriented, gram-scale synthesis of 1 (FIG. 5B) began with a Suzuki coupling between pyrimidine 140 and boronic ester 142 to deliver the azaindole pyrimidine core 139. Utilizing method A, sulfoximine 138 and pyrimidine 139 gratifyingly provided the congested pyrimidine core of 1 in a single step on multi-gram scale (109, 79% yield, >3 g prepared in a single flask). A one-pot t-butyl cleavage/S_{N}Ar sequence with morpholine 131 gave rise to protected sulfinamide 143 in 88% yield. The desired methyl sulfoximine arose from an S-alkylation with methyl iodide to afford 144. Lastly, bis-deprotection of N-Piv and N-Bn was realized after extensive reaction screening—various reductive (Pd using H₂ sources with pressures up to 100 psi as well as Na° reductions) and acid mediated hydrolytic (AlCl₃, FeCl₃, FeBr₃) conditions were all explored. A two-step optimized deprotection via acid hydrolysis of N-Piv followed by oxidative N-Bn cleavage resulted in 1 (>1 g) with an 87% yield over two steps and one final purification. Further development of protecting group strategy may be required for a more ideal manufacturing route of ceralasertib.

Conversely, the diversity-oriented synthetic route highlights four distinct sites and advanced intermediates that can be exploited for analog development (FIG. 5C). Enantiopure t-Bu pyrimidyl sulfoximine 82 was made accessible on gram-scale from trifunctional pyrimidine 140 in 86% yield using method A. At this stage, an alkylation at the benzylic position, a second S_{N}Ar on the pyrimidine or a cross-coupling are all options. Functionalization of the benzylic position was chosen as the second step via α-kylation with 1,2-dibromoethane (145) to give 83 in good yield (72%). A second S_{N}Ar was achieved using morpholine 131 with good regiocontrol (ca. 9:1) and high yield (91%) at low reaction temperatures—providing advanced intermediate 146 and leaving two diversification sites remaining.

The two step S-alkylation sequence using 2-iodopyrimidine 146 could be employed at this point, leaving the last diversification site at the 2-position of the pyrimidine capable of eastern region analog development. However, we decided to push the boundaries of the t-butyl sulfoximine to determine if it were stable under typical cross-coupling conditions. Gratifyingly, 146 underwent a Suzuki coupling with boronic ester 141 to give fully protected t-Bu sulfoximine 147 in 83% yield. An S-alkylation sequence provided methyl sulfoximine 148 that was subsequently deprotected to give ceralasertib 1, fully demonstrating the feasibility of the diversity-oriented route. The disclosed improvements made to incorporate congested sulfoximine moieties provides an alternative approach to 1 and related scaffolds. By utilizing the S_{N}Ar approach with tri-functional pyrimidine 140, the points of diversity were increased by two, the overall yield increased by 20%, and the step count decreased by two.

### General Experimental

Reagents were purchased at the highest commercial quality and used without further purification, unless otherwise stated. Anhydrous tetrahydrofuran (THF), 1,4-dioxane (dioxane), acetonitrile (MeCN), dichloromethane (DCM), and dimethylformamide (DMF) were obtained by passing the previously degassed solvent through an activated alumina column (PPT Glass Contour Solvent Purification System). Anhydrous dimethylsulfoxide (DMSO) and dimethylacetamide (DMA) was purchased from Acros Organics. NaHMDS (2M in THF) was purchased from Acros Organics with an AcroSeal™, stored under argon and typically used within 6 months. All glassware was flame-dried under vacuum before use. Yields refer to chromatographically and spectroscopically (¹H NMR) homogeneous material, unless otherwise stated. Reactions were monitored by LC-MS or thin layer chromatography (TLC) carried out on 250 μm SiliCycle SiliaPlates (TLC Glass-Backed TLC Extra Hard Layer, 60 Å), using shortwave UV light as the visualizing agent and p-anisaldehyde, phosphomolybdic acid (PMA) or KMnO₄ with heat as developing agents.

Flash column chromatography was performed with a Biotage Isolera One (ZIP or SNAP Ultra cartridges) or with traditional glass flash columns using SiliCycle SiliaFlash® P60 (particle size 40-63 μm). NMR spectra were recorded on a Bruker Ascend™ 500 MHz instrument or Bruker Neo600 600 MHz spectrometer and were calibrated using residual undeuterated solvent as an internal reference (CDCl3: 7.26 ppm ¹H NMR, 77.16 ppm ¹³C NMR; DMSO-d₆: 2.50 ppm ¹H NMR, 39.5 ppm ¹³C NMR). The following abbreviations were used to explain NMR peak multiplicities: s=singlet, d=doublet, t=triplet, q=quartet, dd=doublet of doublet, ddd=doublet of doublet of doublet, dddd=doublet of doublet of doublet of doublet, ddddd=doublet of doublet of doublet of doublet of doublet, tt=triplet of triplet, ddt=doublet of doublet of triplet, m=multiplet, br=broad, hept=heptet. High resolution mass spectra (HRMS) were recorded on an Agilent 6230 LC-MS TOF mass spectrometer. Enantiomeric excess (ee) was determined using a Varian Prostar HPLC with a 210 binary pump and a 335 diode array detector. Optical rotations were measured using a JASCO P-2000 polarimeter with a cell length of 1 dm. Melting points were recorded on a Chemglass DMP 100 melting point apparatus and were uncorrected.

### Handling of Reagents

All synthesized sulfonimidoyl nucleophiles (except t-Bu sulfoximines) and heterocycles were stored under ambient conditions. Sulfoximines and S_{N}Ar products containing t-Bu substituents were stored at −80° C. It is important to note that t-Bu sulfoximines stored at room temperature performed equally as well as those stored at −80° C., however, most S_{N}Ar products containing t-Bu substituents and α-H decomposed (e.g. compounds 52 and 82) over 14 days at room temperature. All intermediates for targeted syntheses and medicinal chemistry routes were stored at −20° C.

### Reaction Optimization

TABLE 1
Reaction optimization of sulfonimidoyl S_{N}Ar

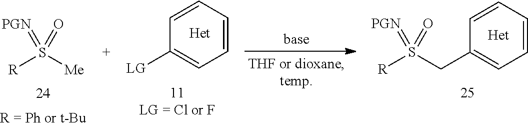

| Entry | NPG | Het | Base (eq.) | Temperature | % yield^{a} |
|---|---|---|---|---|---|
| 1 | NBz | pyrimidine^{b} | NaHMDS | −78° C. to rt | 55% |
| | | | (1.1) | | |
| 2 | NBz | pyrimidine^{b} | NaHMDS | −78° C. to rt | 91% |
| | | | (2.2) | | |
| 3 | NBz | pyridine^{c} | NaHMDS | −78° C. to rt | 0% |
| | | | (2.2) | | |
| 4 | NTs | pyridine^{c} | NaHMDS | −78° C. to rt | 32% |
| | | | (2.2) | | |
| 5 | NTs | pyridine^{c} | NaH (2.2), | rt | 0% |
| | | | 15-crown-5 | | |
| 6 | NTs | pyridine^{c} | NaH (2.2), | rt to 80° C. | 81% |
| | | | 15-crown-5 | | |
| 7 | NBz | pyrimidine^{b} | K₂CO₃ | rt to 80° C. | 0% |
| 8 | NBz | pyrimidine^{b} | Cs₂CO₃ | rt to 80° C. | 0% |
| 9 | NBz | pyrimidine^{b} | LiHMDS (2.2) | −78° C. to rt | 66% |
| 10 | NBz | pyrimidine^{b} | KHMDS (2.) | −78° C. to rt | 72% |
| 11 | NBz | pyrimidine^{b} | KOt-Bu | rt to 80° C. | 33%^{d} |
| 12 | NTs | pyridine^{c} | NaH (2.2) | rt to 80° C. | 31% |

All reactions were performed on 0.25 mmol scale. ªIsolated yields. ^{b}4,6-dichloro-2-(methylthio)pyrimidine was used. ^{c}2-fluoropyridine was used. ^{d}Yield determined by LC-MS. THF was used as the solvent when cryogenic conditions were used. Dioxane was used as the solvent when heating was required.

### Sulfonimidoyl S_{N}Ar Scope:

### General S_{N}Ar Procedure for Method A (GP-4):

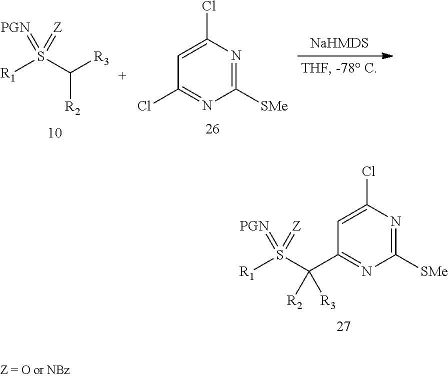

In a septum capped 2-dram reaction vial or culture vial (for 0.25 mmol scale) equipped with a stir bar and argon balloon was added 10 (1 eq.), 26 (1 eq.) and THF (0.1 M) then cooled to −78° C. NaHMDS (2M in THF, 2.2 eq. for 10 and 2° 10 and 1.1-1.5 eq. for 3° 10) was added then stirred at −78° C. for 1 hour before gradually warming to room temperature (not all substrates require warming to room temperature, vide infra). Once complete (monitored by TLC and LC-MS), reactions were quenched with saturated aqueous NH₄Cl and water, extracted with EtOAc (4 times), combined organic layers dried over Na₂SO₄, filtered and concentrated. Further purification by silica gel column chromatography using hexanes/EtOAc provided the desired sulfonimidoyl S_{N}Ar products (27). Note: All reactions were performed on 0.25 mmol scales unless otherwise stated. Pyrimidine 26 was purchased from Combi-Blocks (red powder) and was purified by silica gel column chromatography using hexanes/EtOAc (10% EtOAc, isocratic) to give a clear crystalline solid prior to use.

### Reaction Monitoring for Method A:

All reactions following GP-4 were set-up as described above and monitored at different time-points by taking aliquots via syringe at 15 minutes, 30 minutes and 1 hour (occasionally longer). If the reaction did not progress after 1 hour at −78° C., it was typically warmed to −40° C. then to room temperature while aliquots were taken at −40° C., 0° C. and room temperature. Each aliquot was analyzed by TLC and LC-MS to determine reaction progression. It is important to note that the aliquot temperature increases once removed from the reaction vial before quenching, which could affect reaction progress monitoring. When a reaction was deemed complete, the reaction vial was typically removed from the dry ice bath before quenching with saturated aqueous NH₄Cl, but not always warmed to room temperature (vide infra).

### General S_{N}Ar Procedure for Method B (GP-5):

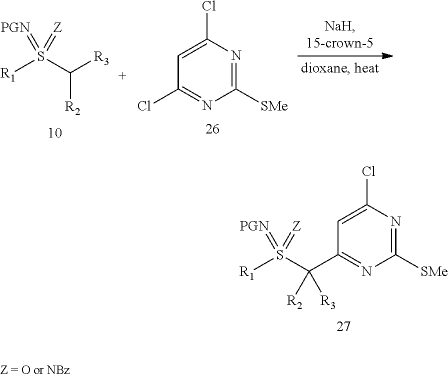

In a septum capped 2-dram reaction vial (for 0.25 mmol scale) equipped with a stir bar and argon balloon was added 10 (1 eq.), 26 (1 eq.), 15-crown-5 ether (2.2 eq. for 1° and 2° 10 and 1.1 eq. for 3° 10) and dioxane (0.1 M). NaH (2.2 eq. for 1° and 2° 10 and 1.1 eq. for 3° 10) was added then stirred at room temperature for 3 minutes before heating to 50-80° C. (depending on the substrate). Once complete (monitored by TLC and LC-MS), reactions were quenched using saturated aqueous NH₄Cl and water. Aqueous layer extracted with EtOAc (4 times), combined organic layers dried over Na₂SO₄, filtered and concentrated. Further purification by silica gel column chromatography using hexanes/EtOAc or DCM/MeOH provided the desired sulfonimidoyl S_{N}Ar products. Note: All reactions were performed on 0.25 mmol scales unless otherwise stated. When N-Ts sulfoximine was used, the mixture was heated to 60° C. to fully dissolve the sulfoximine prior to the addition of NaH.

### Reaction Monitoring for Method B:

All reactions following GP-5 were set-up as described above and monitored at different time-points by taking aliquots via syringe at 1 hour, 3 hours and 15 hours (occasionally longer). Each aliquot was analyzed by TLC and LC-MS to determine reaction progression. Initial attempts were usually conducted at 60° C.—temperature was elevated to increase reaction progression if required.

### Nucleophile Scope:

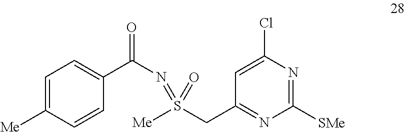 [SMILES: CSc1nc(Cl)cc(CS(C)(=O)=NC(=O)c2ccc(C)cc2)n1]

GP-4 was used (−78° C. for 1 h, warmed to room temperature over 1 hour). Purified by silica gel column chromatography using hexanes/EtOAc (0% to 40% EtOAc gradient) to give 28 (3.72 g, 10.1 mmol, 93% yield) as an amorphous solid. Note: NaHMDS was added over two minutes. *Crude sulfoximine provided 28 (0.582 g, 1.57 mmol, 76% yield).

TLC: R_{f}=0.4 (hexanes/EtOAc, 20% EtOAc, UV). ¹H NMR: (500 MHz, CDCl₃) δ 7.96 (d, J=8.2 Hz, 2H), 7.21 (d, J=7.0 Hz, 3H), 4.97 (d, J=13.4 Hz, 1H), 4.83 (d, J=13.4 Hz, 1H), 3.36 (s, 3H), 2.55 (s, 3H), 2.40 (s, 3H) ppm. ¹³C NMR: (126 MHz, CDCl₃) δ 174.30, 162.21, 158.55, 143.11, 132.34, 129.41, 128.86, 118.25, 59.91, 40.52, 21.65, 14.36 ppm. HRMS: Calc'd for C₁₅H₁₇ClN₃O₂S₂ [M+H⁺] 370.0445; found: 370.0446.

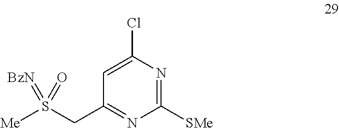

GP-4 was used (−78° C. for 1 h, warmed to −40° C. over 30 minutes). Purified by silica gel column chromatography using hexanes/EtOAc (0% to 30% EtOAc gradient) to give 29 (0.078 g, 0.219 mmol, 88% yield) as a clear colorless oil

TLC: R_{f}=0.22 (hexanes/EtOAc, 30% EtOAc, UV). ¹H NMR: (600 MHz, CDCl₃) δ 8.09-8.04 (m, 2H), 7.55-7.49 (m, 1H), 7.40 (t, J=7.7 Hz, 2H), 7.21 (s, 1H), 4.97 (d, J=13.4 Hz, 1H), 4.84 (d, J=13.4 Hz, 1H), 3.38 (s, 3H), 2.54 (s, 3H) ppm. ¹³C NMR: (151 MHz, CDCl₃) δ 174.37, 162.25, 158.46, 134.99, 132.49, 129.33, 128.13, 118.24, 59.87, 40.52, 14.35 ppm. HRMS: Calc'd for C₁₄H₁₅ClN₃O₂S₂ [M+H⁺] 356.0289; found: 356.0289.

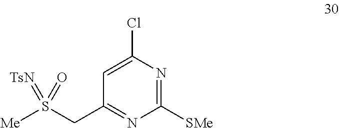

GP-4 was used (−78° C. for 1.5 hours, warmed to room temperature then stirred for 3 hours). Purified by silica gel column chromatography using hexanes/EtOAc (0% to 40% EtOAc gradient) to give 30 (0.087 g, 0.214 mmol, 86% yield) as a clear colorless oil that solidified upon standing.

TLC: R_{f}=0.75 (hexanes/EtOAc, 60% EtOAc, UV)¹H NMR: (500 MHz, CDCl₃) δ 7.83 (d, J=8.3 Hz, 2H), 7.34 (s, 1H), 7.28 (d, J=8.1 Hz, 2H), 4.88 (d, J=13.7 Hz, 1H), 4.84 (d, J=13.7 Hz, 1H), 3.27 (s, 3H), 2.56 (s, 3H), 2.41 (s, 3H) ppm. ¹³C NMR: (126 MHz, CDCl₃) δ 174.37, 162.60, 157.95, 143.31, 140.11, 129.42, 126.62, 118.64, 61.90, 42.02, 21.58, 14.38 ppm. HRMS: Calc'd for C₁₄H₁₇ClN₃O₂S₃ [M+H⁺] 406.0115; found: 406.0120.

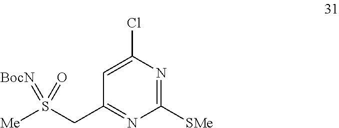

GP-4 was used (−78° C. for 1 hour, warmed to 0° C. then stirred for 1 hour). Purified by silica gel column chromatography using hexanes/EtOAc (0% to 40% EtOAc gradient) to give 31 (0.081 g, 0.230 mmol, 92% yield) as a clear colorless oil that solidified upon standing.

TLC: R_{f}=0.47 (hexanes/EtOAc, 40% EtOAc, UV)¹H NMR: (500 MHz, CDCl₃) δ 7.19 (s, 1H), 4.99 (d, J=13.5 Hz, 1H), 4.73 (d, J=13.4 Hz, 1H), 3.20 (s, 3H), 2.55 (s, 3H), 1.49 (s, 9H) ppm. ¹³C NMR: (126 MHz, CDCl₃) δ 174.41, 162.39, 158.75, 158.30, 118.01, 81.17, 59.03, 40.72, 28.07, 14.35 ppm. HRMS: Calc'd for C₁₂H₁₈ClN₃O₃S₂Na [M+Na⁺] 374.0370; found: 374.0374.

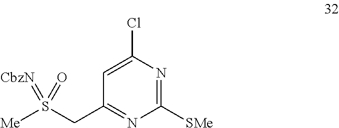

GP-4 was used (−78° C. for 1 hour, warmed to 0° C. then stirred for 2 hour). Purified by silica gel column chromatography using hexanes/EtOAc (0% to 40% EtOAc gradient) to give 32 (0.082 g, 0.213 mmol, 85% yield) as a clear colorless oil that solidified upon standing.

TLC: R_{f}=0.34 (hexanes/EtOAc, 40% EtOAc, UV)¹H NMR: (500 MHz, CDCl₃) δ 7.36 (d, J=7.1 Hz, 5H), 7.10 (s, 1H), 5.18-5.10 (m, 2H), 4.83 (d, J=13.6 Hz, 1H), 4.77 (d, J=13.6 Hz, 1H), 3.25 (s, 3H), 2.55 (s, 3H) ppm. ¹³C NMR: (126 MHz, CDCl₃) δ 174.43, 162.45, 158.77, 158.34, 135.87, 128.58, 128.30, 128.28, 118.01, 68.19, 59.26, 40.65, 14.36 ppm. HRMS: Calc'd for C₁₅H₁₇ClN₃O₃S₂ [M+H⁺] 386.0394; found: 386.0391.

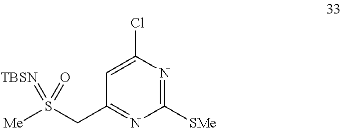

GP-4 was used (−78° C. for 1.5 h, warmed to room temperature then stirred for 4 hours). Purified by silica gel column chromatography using hexanes/EtOAc (0% to 20% EtOAc gradient) to give 33 (0.073 g, 0.199 mmol, 80% yield) as a light-yellow oil.

TLC: R_{f}=0.36 (hexanes/EtOAc, 20% EtOAc, UV). ¹H NMR: (500 MHz, CDCl₃) δ 7.12 (s, 1H), 4.23 (dd, J=13.1, 0.8 Hz, 1H), 4.14 (dd, J=13.0, 0.9 Hz, 1H), 2.92 (t, J=0.8 Hz, 3H), 2.56 (s, 3H), 0.85 (s, 9H), 0.06 (d, J=4.6 Hz, 6H) ppm. ¹³C NMR: (126 MHz, CDCl₃) δ 173.76, 161.47, 161.00, 117.82, 66.24, 45.61, 25.81, 17.83, 14.33, −2.38, −2.47 ppm. HRMS: Calc'd for C₁₃H₂₅ClN₃OS₂Si [M+H⁺] 366.0891; found: 366.0895.

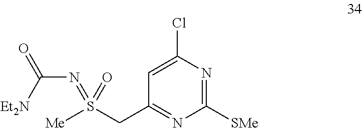

GP-4 was used (−78° C. for 1 h, warmed to room temperature then stirred for 1.5 hours). Purified by silica gel column chromatography using hexanes/EtOAc (0% to 40% EtOAc gradient) to give 34 (0.082 g, 0.233 mmol, 93% yield) as a clear colorless oil.

TLC: R_{f}=0.24 (hexanes/EtOAc, 40% EtOAc, UV). ¹H NMR: (500 MHz, CDCl₃) δ 7.18 (s, 1H), 4.94 (d, J=13.1 Hz, 1H), 4.68 (d, J=13.2 Hz, 1H), 3.34-3.15 (m, 4H), 3.11 (s, 3H), 2.50 (s, 3H), 1.06 (t, J=6.5 Hz, 3H), 0.94 (t, J=7.1 Hz, 3H) ppm. ¹³C NMR: (126 MHz, CDCl₃) δ 173.96, 162.02, 159.63, 159.17, 118.17, 59.99, 42.33, 40.82, 40.43, 14.35, 13.85, 13.53 ppm. HRMS: Calc'd for C₁₂H₂₀ClN₄O₂S₂ [M+H⁺] 351.0711; found: 351.0710.

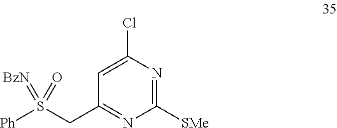

GP-4 was used (−78° C. for 1 h, warmed to 0° C. then stirred for 1.5 hours). Purified by silica gel column chromatography using hexanes/EtOAc (0% to 20% EtOAc gradient) to give 35 (0.095 g, 0.227 mmol, 91% yield) as a clear colorless oil.

TLC: R_{f}=0.43 (hexanes/EtOAc, 40% EtOAc, UV). ¹H NMR: (500 MHz, CDCl₃) δ 8.16 (dd, J=8.4, 1.4 Hz, 2H), 7.84 (dd, J=8.5, 1.3 Hz, 2H), 7.74-7.67 (m, 1H), 7.61-7.52 (m, 3H), 7.44 (t, J=7.7 Hz, 2H), 7.12 (s, 1H), 5.07 (d, J=13.0 Hz, 1H), 5.02 (d, J=12.9 Hz, 1H), 2.15 (d, J=0.8 Hz, 3H) ppm. ¹³C NMR: (126 MHz, CDCl₃) δ 174.58, 173.79, 161.55, 158.42, 135.60, 135.30, 134.38, 132.57, 129.49, 129.39, 128.64, 128.19, 117.97, 62.05, 13.89 ppm. HRMS: Calc'd for C₁₉H₁₇ClN₃O₂S₂ [M+H⁺] 418.0445; found: 418.0445.

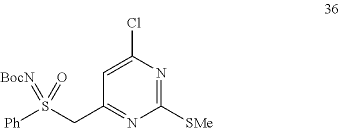

GP-4 was used (−78° C. for 1 h, warmed to 0° C. then stirred for 2 hours). Purified by silica gel column chromatography using hexanes/EtOAc (0% to 35% EtOAc gradient) to give 36 (0.084 g, 0.203 mmol, 81% yield) as a clear colorless oil that solidified upon standing.

TLC: R_{f}=0.59 (hexanes/EtOAc, 40% EtOAc, UV). ¹H NMR: (500 MHz, CDCl₃) δ 7.77 (dd, J=8.5, 1.2 Hz, 2H), 7.69-7.65 (m, 1H), 7.57-7.51 (m, 2H), 4.96 (d, J=13.1 Hz, 1H), 4.78 (d, J=13.1 Hz, 1H), 2.16 (s, 3H), 1.47 (s, 9H) ppm. ¹³C NMR: (126 MHz, CDCl₃) δ 173.77, 161.63, 158.37, 157.99, 135.35, 134.23, 129.22, 128.83, 117.88, 81.25, 61.79, 28.08, 13.89 ppm. HRMS: Calc'd for C₁₇H₂₀ClN₃O₃S₂Na [M+Na⁺] 436.0527; found: 436.0530.

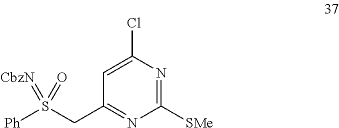

GP-4 was used (−78° C. for 1 h, warmed to 0° C. then stirred for 1.5 hours). Purified by silica gel column chromatography using hexanes/EtOAc (0% to 40% EtOAc gradient) to give 37 (0.101 g, 0.225 mmol, 90% yield) as a clear colorless oil.

TLC: R_{f}=0.57 (hexanes/EtOAc, 40% EtOAc, UV). ¹H NMR: (500 MHz, CDCl₃) δ 7.75 (dd, J=8.5, 1.1 Hz, 2H), 7.69-7.65 (m, 1H), 7.55-7.50 (m, 2H), 7.39-7.27 (m, 5H), 5.19 (d, J=12.3 Hz, 1H), 5.14 (d, J=12.3 Hz, 1H), 4.90 (d, J=13.2 Hz, 1H), 4.84 (d, J=13.2 Hz, 1H), 2.17 (s, 3H) ppm. ¹³C NMR: (126 MHz, CDCl₃) δ 173.86, 161.65, 158.74, 158.05, 135.99, 134.92, 134.48, 129.34, 128.78, 128.53, 128.30, 128.20, 117.83, 68.27, 61.86, 13.91 ppm. HRMS: Calc'd for C₂₀H₁₈ClN₃O₃S₂Na [M+Na⁺] 470.0370; found: 470.0373.

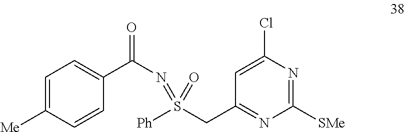 [SMILES: CSc1nc(Cl)cc(CS(=O)(=NC(=O)c2ccc(C)cc2)c2ccccc2)n1]

GP-4 was used (−78° C. for 25 minutes, warmed to room temperature then stirred for 15 minutes). Purified by silica gel column chromatography using hexanes/EtOAc (0% to 20% EtOAc gradient) to give 38 (1.92 g, 4.44 mmol, 79% yield) as a clear colorless oil.

TLC: R_{f}=0.28 (hexanes/EtOAc, 20% EtOAc, UV). ¹H NMR: (500 MHz, CDCl₃) δ 8.05 (d, J=8.2 Hz, 2H), 7.84 (dd, J=8.5, 1.2 Hz, 2H), 7.70 (ddt, J=8.6, 7.0, 1.2 Hz, 1H), 7.60-7.54 (m, 2H), 7.25-7.21 (m, 2H), 7.14 (s, 1H), 5.07 (d, J=13.0 Hz, 1H), 5.02 (d, J=13.0 Hz, 1H), 2.42 (s, 3H), 2.16 (s, 3H) ppm. ¹³C NMR: (126 MHz, CDCl₃) δ 174.61, 173.75, 161.56, 158.50, 143.21, 135.71, 134.31, 132.64, 129.58, 129.35, 128.91, 128.65, 117.97, 62.10, 21.68, 13.89 ppm. HRMS: Calc'd for C₂₀H₁₉ClN₃O₂S₂ [M+H⁺] 432.0602; found: 432.0602.

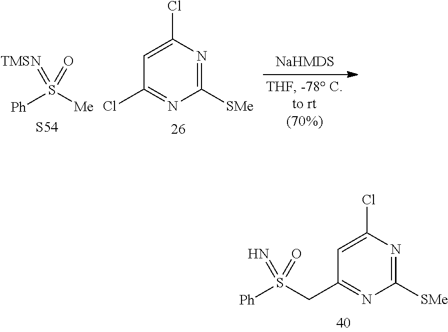

In a septum capped 2-dram reaction vial equipped with a stir bar and argon balloon was added S54 (39) (57.0 mg, 0.25 mmol, 1 eq.), 26 (49.0 mg, 0.25 mmol, 1 eq.) and THF (2.5 mL) then cooled to −78° C. NaHMDS (0.275 mL, 0.550 mmol, 2M in THF, 2.2 eq.) was added dropwise and stirred at −78° C. for 1 hour then gradually warmed to room temperature where it stirred for 30 minutes. Quenched with saturated NH₄Cl (8 mL) and H2O (5 mL), extracted with EtOAc (4×15 mL), combined organic layer dried over Na₂SO₄, filtered and concentrated. Further purification using hexanes/EtOAc (0% to 40%) provided 40 (55.0 mg, 0.175 mmol, 70% yield) as a clear colorless oil that solidified upon standing. Note: Full TMS deprotection occurs during work-up and column chromatography.

TLC: R_{f}=0.36 (hexanes/EtOAc, 50% EtOAc, UV). ¹H NMR: (500 MHz, CDCl₃) δ 7.85-7.81 (m, 2H), 7.67-7.62 (m, 1H), 7.56-7.51 (m, 2H), 7.10 (s, 1H), 4.68 (s, 1H), 4.45 (d, J=12.9 Hz, 1H), 4.42 (d, J=13.0 Hz, 1H), 3.69 (s, 1H), 2.59 (s, 1H), 2.29 (s, 3H) ppm. ¹³C NMR: (126 MHz, CDCl₃) δ 173.71, 161.34, 159.48, 139.89, 133.66, 129.14, 128.87, 117.65, 65.14, 14.05 ppm. HRMS: Calc'd for C₁₂H₁₃ClN₃OS₂ [M+H⁺] 314.0183; found: 314.0181.

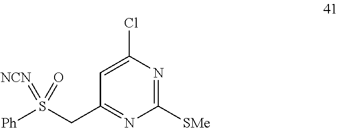

GP-4 was used (−78° C. for 1 h, warmed to room temperature then stirred for 30 minutes). Purified by silica gel column chromatography using hexanes/EtOAc (0% to 60% EtOAc gradient) to give 41 (0.073 g, 0.215 mmol, 86% yield) as a clear colorless oil.

TLC: R_{f}=0.76 (hexanes/EtOAc, 60% EtOAc, UV). ¹H NMR: (500 MHz, CDCl₃) δ 7.82-7.75 (m, 3H), 7.67-7.61 (m, 2H), 7.13 (s, 1H), 4.70 (s, 2H), 2.24 (s, 3H) ppm. ¹³C NMR: (126 MHz, CDCl₃) δ 174.44, 162.04, 156.42, 135.92, 133.08, 130.01, 129.19, 117.99, 111.22, 63.14, 14.03 ppm. HRMS: Calc'd for C₁₃H₁₁ClN₄OS₂ [M+H⁺] 339.0136; found: 339.0135.

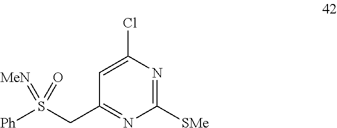

GP-4 was used with methyl(methylimino) phenyl sulfoximine²⁹ (−78° C. for 1.5 h, warmed to room temperature then stirred for 45 minutes). Purified by silica gel column chromatography using hexanes/EtOAc (0% to 40% EtOAc gradient) to give⁴² (0.078 g, 0.237 mmol, 95% yield) as a light-yellow oil.

TLC: R_{f}=0.28 (hexanes/EtOAc, 20% EtOAc, UV). ¹H NMR: (500 MHz, CDCl₃) δ 7.72-7.68 (m, 2H), 7.66-7.58 (m, 1H), 7.55-7.49 (m, 2H), 7.05 (s, 1H), 4.46 (s, 2H), 2.81 (s, 3H), 2.25 (s, 3H) ppm. ¹³C NMR: (126 MHz, CDCl₃) δ 173.52, 161.16, 159.49, 136.36, 133.48, 129.55, 129.27, 117.70, 62.69, 29.67, 14.00 ppm. HRMS: Calc'd for C₁₃H₁₅ClN₃OS₂ [M+H⁺] 328.0340; found: 328.0343.

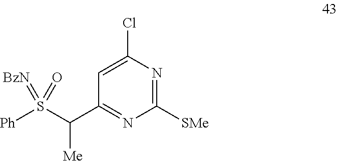

GP-4 was used (−78° C. for 1 h, warmed to −40° C. over 30 minutes then quenched). Purified by silica gel column chromatography using hexanes/EtOAc (0% to 30% EtOAc gradient) to give 43 (0.103 g, 0.238 mmol, 95% yield, d.r. 1:1 determined by LC-MS) as a clear colorless oil.

TLC: R_{f}=0.46 (hexanes/EtOAc, 40% EtOAc, UV) *Both diastereomers ¹H NMR: (500 MHz, CDCl₃) δ 8.18-8.15 (m, 1H), 8.13-8.07 (m, 1H), 7.74-7.65 (m, 3H), 7.59-7.50 (m, 3H), 7.47-7.39 (m, 2H), 7.34-7.11 (m, 1H), 5.36-5.27 (m, 1H), 2.22-2.12 (m, 3H), 1.85-1.68 (m, 3H) ppm. ¹³C NMR: (126 MHz, CDCl₃) δ 174.21, 173.38, 162.78, 161.29, 135.42, 134.26, 133.25, 132.44, 129.58, 129.43, 129.08, 128.15, 117.83, 65.98, 13.91, 12.90 ppm. *NMR analysis of both diastereomers combined. HRMS: Calc'd for C₂₀H₁₉ClN₃O₂S₂ [M+H⁺] 432.0602; found: 432.0605.

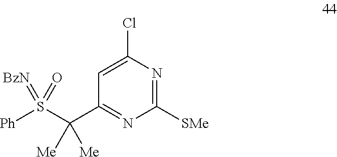

GP-4 was used (−78° C. for 30 minutes then quenched). Purified by silica gel column chromatography using hexanes/EtOAc (0% to 25% EtOAc gradient) to give 44 (0.101 g, 0.226 mmol, 91% yield) as a white solid. Note: 1.5 eq. of NaHMDS was used. When 2.2 eq. of NaHMDS was used, the yield was 77%.

TLC: R_{f}=0.53 (hexanes/EtOAc, 40% EtOAc, UV). ¹H NMR: (500 MHz, CDCl₃) δ 8.07 (d, J=8.1 Hz, 2H), 7.66-7.60 (m, 1H), 7.54-7.45 (m, 6H), 7.42 (t, J=7.7 Hz, 2H), 2.14 (s, 3H), 2.04 (s, 3H), 1.84 (s, 3H) ppm. ¹³C NMR: (126 MHz, CDCl₃) δ 173.62, 172.64, 165.99, 161.30, 135.72, 133.99, 132.80, 132.25, 130.01, 129.37, 129.02, 128.14, 116.91, 69.20, 21.02, 20.45, 13.96 ppm. HRMS: Calc'd for C₂₁H₂₁ClN₃O₂S₂ [M+H⁺] 446.0758; found: 446.0754.

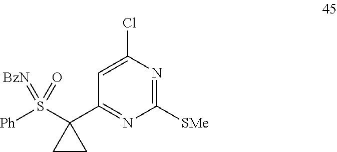

GP-4 was used (−78° C. for 15 minutes, warmed to 0° C. then stirred for 15 minutes). Purified by silica gel column chromatography using hexanes/EtOAc (0% to 25% EtOAc gradient) to give 45 (0.088 g, 0.198 mmol, 79% yield) as a white solid. Note: 1.3 eq. of NaHMDS was used.

TLC: R_{f}=0.29 (hexanes/EtOAc, 20% EtOAc, UV).

¹H NMR: (500 MHz, CDCl₃) δ 8.11-8.06 (m, 2H), 7.74 (dd, J=8.6, 1.3 Hz, 2H), 7.65 (ddt, J=7.9, 7.0, 1.2 Hz, 1H), 7.55-7.49 (m, 3H), 7.45-7.40 (m, 2H), 7.39 (s, 1H), 2.48 (ddd, J=10.6, 7.8, 5.6 Hz, 1H), 2.23 (s, 3H), 2.08 (ddd, J=10.6, 7.7, 5.5 Hz, 1H), 1.75 (ddd, J=9.6, 7.6, 5.7 Hz, 1H), 1.52 (ddd, J=9.6, 7.8, 5.5 Hz, 1H). ¹³C NMR: (126 MHz, CDCl₃) δ 173.51, 163.30, 161.22, 136.55, 135.53, 133.88, 132.35, 129.39, 129.27, 128.65, 128.15, 118.76, 47.58, 15.52, 13.99, 13.49. HRMS: Calc'd for C₂₁H₁₉ClN₃O₂S₂ [M+H⁺]444.0602; found: 444.0606.

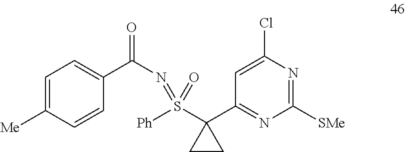 [SMILES: CSc1nc(Cl)cc(C2(S(=O)(=NC(=O)c3ccc(C)cc3)c3ccccc3)CC2)n1]

GP-4 was used (−78° C. for 45 minutes, removed from dry ice bath for 15 minutes then quenched). Purified by silica gel column chromatography using hexanes/EtOAc (0% to 30% EtOAc gradient) to give 46 (0.092 g, 0.201 mmol, 80% yield) as a white solid. Note: 1.3 eq. of NaHMDS was used.

TLC: R_{f}=0.44 (hexanes/EtOAc, 40% EtOAc, UV). ¹H NMR: (500 MHz, CDCl₃) δ 7.98 (d, J=8.2 Hz, 2H), 7.75-7.71 (m, 2H), 7.64 (t, J=7.5 Hz, 1H), 7.52 (t, J=7.9 Hz, 2H), 7.41 (s, 1H), 7.22 (d, J=7.9 Hz, 2H), 2.47 (ddd, J=10.7, 7.8, 5.7 Hz, 1H), 2.41 (s, 3H), 2.23 (s, 3H), 2.07 (ddd, J=10.7, 7.7, 5.6 Hz, 1H), 1.74 (ddd, J=9.5, 7.6, 5.6 Hz, 1H), 1.52 (ddd, J=9.6, 7.8, 5.5 Hz, 1H) ppm. ¹³C NMR: (126 MHz, CDCl₃) δ 173.53, 173.36, 163.39, 161.20, 142.95, 136.66, 133.83, 132.89, 129.49, 129.25, 128.89, 128.66, 118.82, 47.57, 21.65, 15.51, 14.00, 13.46 ppm. HRMS: Calc'd for C₂₂H₂₁ClN₃O₂S₂ [M+H⁺]458.0758; found: 458.0760.

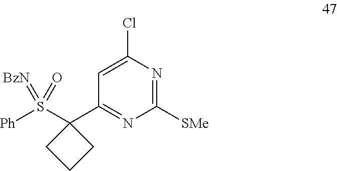

GP-4 was used (−78° C. for 1 hour, warmed to −40° C. then quenched). Purified by silica gel column chromatography using hexanes/EtOAc (0% to 20% EtOAc gradient) to give 47 (0.086 g, 0.178 mmol, 75% yield) as a white solid. Note: 1.2 eq. of NaHMDS was used.

TLC: R_{f}=0.39 (hexanes/EtOAc, 30% EtOAc, UV). ¹H NMR: (500 MHz, CDCl₃) δ 8.10-8.06 (m, 2H), 7.65-7.57 (m, 1H), 7.54-7.50 (m, 1H), 7.48-7.40 (m, 6H), 7.37 (s, 1H), 3.64-3.56 (m, 1H), 3.19 (dddt, J=12.6, 10.1, 7.8, 1.2 Hz, 1H), 2.84 (ddddd, J=13.1, 9.2, 5.2, 2.7, 1.1 Hz, 1H), 2.66 (ddddd, J=13.1, 9.3, 5.3, 2.7, 1.1 Hz, 1H), 2.29 (dddd, J=15.3, 11.3, 10.2, 5.3 Hz, 1H), 2.10 (s, 3H), 2.01-1.90 (m, 1H) ppm. ¹³C NMR: (126 MHz, CDCl₃) δ 174.01, 172.77, 166.38, 160.98, 135.62, 133.91, 133.16, 132.27, 129.36, 129.29, 129.04, 128.14, 71.10, 29.65, 27.78, 16.00, 13.88 ppm. HRMS: Calc'd for C₂₂H₂₁ClN₃O₂S₂[M+H⁺] 458.0758; found: 458.0752.

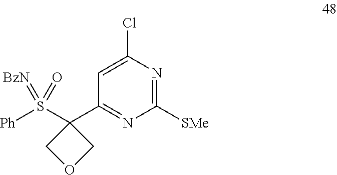

GP-4 was used (−78° C. for 1 hour, warmed to 0° C. then stirred for 30 minutes). Purified by silica gel column chromatography using hexanes/EtOAc (0% to 25% EtOAc gradient) to give 48 (0.082 g, 0.178 mmol, 71% yield) as an amorphous solid. Note: 1.2 eq. of NaHMDS was used. Longer reaction time may increase overall yield.

TLC: R_{f}=0.32 (hexanes/EtOAc, 30% EtOAc, UV). ¹H NMR: (500 MHz, CDCl₃) δ 8.07 (dd, J=8.3, 1.4 Hz, 2H), 7.73-7.68 (m, 1H), 7.62-7.57 (m, 2H), 7.57-7.51 (m, 3H), 7.42 (t, J=7.8 Hz, 2H), 7.24 (s, 1H), 5.95 (d, J=8.0 Hz, 1H), 5.42 (d, J=7.6 Hz, 1H), 5.17 (d, J=8.7 Hz, 1H), 4.94 (d, J=6.9 Hz, 1H), 2.10 (s, 3H) ppm. ¹³C NMR: (126 MHz, CDCl₃) δ 174.41, 173.40, 163.94, 161.71, 134.89, 134.65, 132.71, 132.42, 129.66, 129.47, 129.29, 128.25, 116.25, 75.31, 74.39, 71.39, 13.87 ppm. HRMS: Calc'd for C₂₁H₁₉ClN₃O₃S₂ [M+H⁺] 460.0551; found: 460.0550.

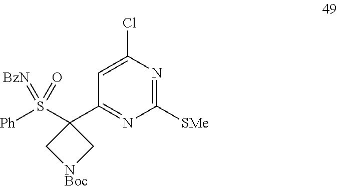

GP-4 was used (−78° C. for 1 hour then quenched). Purified by silica gel column chromatography using hexanes/EtOAc (0% to 25% EtOAc gradient) to give 49 (0.131 g, 0.234 mmol, 94% yield) as an amorphous solid. Note: 1.5 eq. of NaHMDS was used.

TLC: R_{f}=0.53 (hexanes/EtOAc, 40% EtOAc, UV). ¹H NMR: (500 MHz, CDCl₃) δ 8.07 (d, J=7.6 Hz, 2H), 7.72-7.65 (m, 1H), 7.60-7.49 (m, 5H), 7.42 (t, J=7.7 Hz, 2H), 7.26 (s, 1H), 5.27 (d, J=18.6 Hz, 1H), 4.80 (s, 1H), 4.54 (d, J=10.2 Hz, 1H), 4.33 (d, J=10.0 Hz, 1H), 2.10 (s, 3H), 1.47 (s, 9H) ppm. ¹³C NMR: (126 MHz, CDCl₃) δ 174.05, 173.39, 163.45, 161.52, 155.63, 134.92, 134.66, 132.67, 129.57, 129.48, 129.35, 128.24, 116.80, 107.93, 81.01, 67.68, 66.16, 28.32, 13.90, 13.90 ppm. HRMS: Calc'd for C₂₆H₂₈ClN₄O₄S₂ [M+H⁺] 559.1235; found: 559.1231.

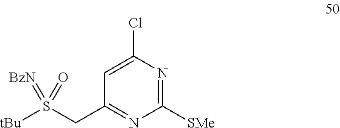

GP-4 was used (−78° C. for 1 hour, warmed to room temperature then stirred for 1 hour). Purified by silica gel column chromatography using hexanes/EtOAc (0% to 20% EtOAc gradient) to give 50 (0.077 g, 0.193 mmol, 77% yield) as a clear colorless oil.

TLC: R_{f}=0.289 (hexanes/EtOAc, 20% EtOAc, UV)¹H NMR: (500 MHz, CDCl₃) δ 8.13 (d, J=7.0 Hz, 2H), 7.55-7.49 (m, 1H), 7.42 (t, J=7.7 Hz, 2H), 5.27 (d, J=13.7 Hz, 1H), 4.80 (d, J=13.7 Hz, 1H), 2.54 (s, 3H), 1.59 (s, 9H) ppm. ¹³C NMR: (126 MHz, CDCl₃) δ 174.97, 173.47, 161.85, 159.91, 135.81, 132.30, 129.31, 128.10, 119.09, 64.06, 52.95, 23.57, 14.29 ppm. HRMS: Calc'd for C₂₀H₂₄ClN₃O₂S₂ [M-tBu+H₊] 398.0758; found: 398.0755.

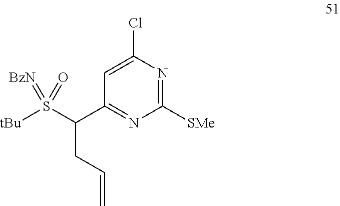

GP-4 was used (−78° C. for 1 hour, warmed to 0° C. over 1 hour then quenched). Purified by silica gel column chromatography using hexanes/EtOAc (0% to 20% EtOAc gradient) to give 51 (0.077 g, 0.176 mmol, 70% yield, d.r. 1.65:1 determined by LC-MS) as a clear colorless oil.

TLC: R_{f}=0.61 (hexanes/EtOAc, 30% EtOAc, UV) and R_{f}=0.44 (hexanes/EtOAc, 30% EtOAc, UV). Major diastereomer: ¹H NMR: (500 MHz, CDCl₃) δ 8.18-8.12 (m, 2H), 7.65 (s, 1H), 7.55-7.50 (m, 1H), 7.43 (t, J=7.7 Hz, 2H), 5.58-5.48 (m, 1H), 5.22 (dd, J=12.1, 3.5 Hz, 1H), 5.07-4.98 (m, 2H), 3.27-3.14 (m, 1H), 3.04-2.96 (m, 1H), 2.56 (s, 3H), 1.55 (s, 9H) ppm. ¹³C NMR: (126 MHz, CDCl₃) δ 173.97, 173.21, 163.47, 161.86, 135.79, 132.21, 131.94, 128.12, 119.49, 118.15, 66.91, 66.20, 33.96, 23.83, 14.35 ppm. HRMS: Calc'd for C₁₆H₁₆ClN₃O₂S₂Na [M-tBu+H⁺] 404.0265; found: 404.0262.

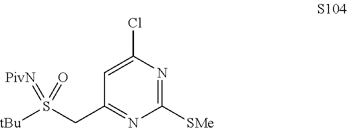

GP-4 was used (−78° C. for 1 hour, warmed to room temperature over 2 hours). Purified by silica gel column chromatography using hexanes/EtOAc (0% to 20% EtOAc gradient) to give S104 (0.081 g, 0.214 mmol, 86% yield) as a clear colorless oil that solidified upon standing.

TLC: R_{f}=0.35 (hexanes/EtOAc, 20% EtOAc, UV). ¹H NMR: (400 MHz, CDCl₃) δ 7.44 (s, 1H), 5.03 (d, J=13.6 Hz, 1H), 4.76 (d, J=13.6 Hz, 1H), 2.53 (s, 3H), 1.47 (s, 9H), 1.19 (s, 9H) ppm. ¹³C NMR: (101 MHz, CDCl₃) δ 189.23, 173.43, 161.77, 160.11, 117.08, 63.85, 52.93, 41.87, 27.66, 23.47, 14.28 ppm. HRMS: Calc'd for C₁₅H₂₄ClN₃O₂S₂Na [M+Na⁺] 400.0891; found: 400.0894. Chiral HPLC analysis: Chiralpak AD-H, hexanes/i-PrOH (97:3), flow rate=1 m/min, k=254 nm; retention time: (S)=9.32 min, (R)=18.97 min.

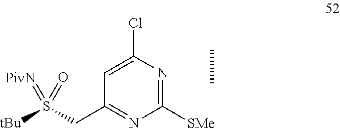

GP-4 was used. (−78° C. for 1 hour, warmed to room temperature over 2 hours). Purified by silica gel column chromatography using hexanes/EtOAc (0% to 20% EtOAc gradient) to give 52 (0.077 g, 0.203 mmol, 81% yield) as a clear colorless oil that solidified upon standing. Gram-scale (2.15 g, 6.31 mmol, 90% yield). Note: Scale-up does not affect reaction time.

TLC: R_{f}=0.34 (hexanes/EtOAc, 20% EtOAc, UV). ¹H NMR: (500 MHz, CDCl₃) δ 7.44 (s, 1H), 5.02 (d, J=13.6 Hz, 1H), 4.76 (d, J=13.6 Hz, 1H), 2.53 (s, 3H), 1.47 (s, 9H), 1.19 (s, 9H) ppm. ¹³C NMR: (126 MHz, CDCl₃) δ 189.24, 173.41, 161.76, 160.08, 118.87, 63.85, 52.92, 41.87, 27.67, 23.47, 14.29. ppm. Specific Rotation=−39.4 (c 1.00, CHCl₃) HRMS: Calc'd for C₁₅H₂₄ClN₃O₂S₂Na [M+Na⁺] 400.0891; found: 400.0892. Melting point: 87° C. Chiral HPLC analysis: Chiralpak AD-H, hexanes/i-PrOH (97:3), flow rate=1 mL/min, k=254 nm; retention time: (S)=9.27 min, (R)=not detected.

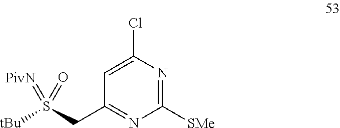

GP-4 was used. (−78° C. for 1 hour, warmed to room temperature over 2 hours). Purified by silica gel column chromatography using hexanes/EtOAc (0% to 20% EtOAc gradient) to give 53 (1.7 g, 4.6 mmol, 84% yield) as a white solid.

TLC: R_{f}=0.37 (hexanes/EtOAc, 20% EtOAc, UV). ¹H NMR: (500 MHz, CDCl₃) δ 7.44 (s, 1H), 5.03 (d, J=13.6 Hz, 1H), 4.77 (d, J=13.6 Hz, 1H), 2.54 (s, 3H), 1.48 (s, 9H), 1.20 (s, 9H) ppm. ¹³C NMR: (126 MHz, CDCl₃) δ 189.21, 173.42, 161.77, 160.09, 118.86, 63.86, 52.94, 41.87, 27.66, 23.48, 14.28 ppm. Specific Rotation=+40.7 (c 1.00, CHCl₃) HRMS: Calc'd for C₁₅H₂₄ClN₃O₂S₂Na [M+Na⁺] 400.0891; found: 400.0894.

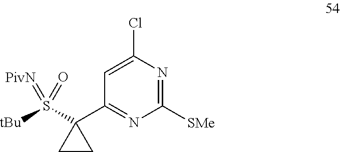

GP-4 was used (−78° C. for 1 hour, warmed to 0° C. then stirred for 30 minutes). Purified by silica gel column chromatography using hexanes/EtOAc (0% to 30% EtOAc gradient) to give 54 (0.076 g, 0.188 mmol, 75% yield) as a white solid.

TLC: R_{f}=0.67 (hexanes/EtOAc, 20% EtOAc, UV). ¹H NMR: (500 MHz, CDCl₃) δ 7.36 (s, 1H), 2.54 (s, 3H), 2.46 (ddd, J=10.5, 8.1, 5.9 Hz, 1H), 1.96 (ddd, J=9.7, 8.2, 5.6 Hz, 1H), 1.83 (ddd, J=10.5, 7.9, 5.5 Hz, 1H), 1.39 (s, 9H), 1.33 (ddd, J=9.7, 5.0, 2.4 Hz, 1H), 1.22 (d, J=0.8 Hz, 9H) ppm. ¹³C NMR: (126 MHz, CDCl₃) δ 187.93, 173.39, 165.49, 161.52, 119.03, 66.69, 42.20, 41.97, 27.78, 24.62, 16.57, 14.48, 14.35 ppm. Specific Rotation=−184.4 (c 1.00, CHCl₃) HRMS: Calc'd for C₁₇H₂₇ClIN₃O₂S [M+H⁺] 426.1047; found: 426.1050.

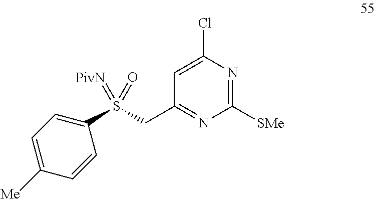

GP-4 was used (−78° C. for 1 hour, warmed to 0° C. then stirred for 30 minutes). Purified by silica gel column chromatography using hexanes/EtOAc (0% to 30% EtOAc gradient) to give 55 (0.088 g, 0.214 mmol, 85% yield) as a clear colorless oil that solidified upon standing.

TLC: R_{f}=0.47 (hexanes/EtOAc, 30% EtOAc, UV). ¹H NMR: (500 MHz, CDCl₃) δ 7.59 (d, J=7.7 Hz, 2H), 7.32 (d, J=8.7 Hz, 2H), 7.08 (s, 1H), 5.06 (d, J=11.9 Hz, 1H), 4.75 (d, J=12.0 Hz, 1H), 2.44 (s, 3H), 2.16 (s, 3H), 1.22 (s, 9H) ppm. ¹³C NMR: (126 MHz, CDCl₃) δ 188.95, 173.56, 161.40, 158.83, 145.42, 132.70, 129.86, 128.62, 117.85, 61.81, 41.87, 27.65, 21.63, 13.77 ppm. Specific Rotation=−100.2 (c 1.00, CHCl₃) HRMS: Calc'd for C₁₈H₂₃ClN₃O₂S₂ [M+H⁺] 412.0915; found: 412.0919.

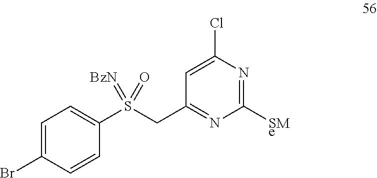

GP-4 was used. (−78° C. for 1 hour, warmed to 0° C. then stirred for 30 minutes). Purified by silica gel column chromatography using hexanes/EtOAc (0% to 20% EtOAc gradient) to give 56 (0.112 g, 0.225 mmol, 90% yield) as a light-yellow oil.

TLC: R_{f}=0.38 (hexanes/EtOAc, 20% EtOAc, UV). ¹H NMR: (500 MHz, Chloroform-d) δ 8.13 (dd, J=8.2, 1.5 Hz, 2H), 7.73-7.67 (m, 4H), 7.57-7.51 (m, 1H), 7.43 (t, J=7.8 Hz, 2H), 7.16 (s, 1H), 5.03 (d, J=13.1 Hz, 1H), 4.99 (d, J=13.1 Hz, 1H), 2.21 (s, 3H). ¹³C NMR: (126 MHz, CDCl₃) δ 174.46, 173.93, 161.75, 158.13, 135.03, 134.73, 132.72, 132.69, 130.15, 130.04, 129.50, 128.23, 118.03, 62.10, 13.82 ppm. HRMS: Calc'd for C₁₉H₁₆BrClN₃O₂S₂ [M+H⁺] 495.9550; found: 495.9554.

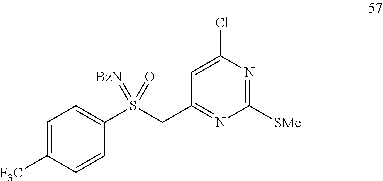

GP-4 was used (−78° C. for 1 hour, warmed to 0° C. then stirred for 1 hour). Purified by silica gel column chromatography using hexanes/EtOAc (0% to 15% EtOAc gradient) to give 57 (0.101 g, 0.208 mmol, 83% yield) as a light-yellow oil.

TLC: R_{f}=0.54 (hexanes/EtOAc, 30% EtOAc, UV). ¹H NMR: (500 MHz, CDCl₃) δ 8.14 (dd, J=8.4, 1.3 Hz, 2H), 7.98 (d, J=8.2 Hz, 2H), 7.84 (d, J=8.3 Hz, 2H), 7.58-7.52 (m, 1H), 7.47-7.41 (m, 2H), 7.19 (s, 1H), 5.03 (d, J=1.2 Hz, 2H), 2.17 (s, 3H) ppm. ¹³C NMR: (126 MHz, CDCl₃) δ 174.44, 174.02, 161.89, 157.86, 139.59, 139.58, 139.47, 136.41, 136.14, 135.92, 135.88, 135.61, 134.90, 134.85, 132.85, 129.54, 129.38, 128.28, 126.51, 126.48, 126.45, 126.42, 126.27, 124.09, 121.92, 119.75, 118.10, 62.03, 13.75 ppm. ¹⁹F NMR: (471 MHz, CDCl₃) δ −63.29 ppm. HRMS: Calc'd for C₂₀H₁₆ClF₃N₃O₂S₂[M+H⁺]486.0319; found: 486.0322.

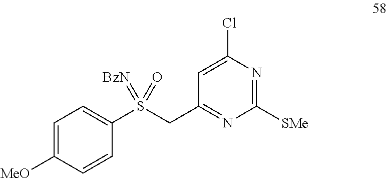

GP-4 was used. (−78° C. for 1 hour, warmed to room temperature over 1 hour). Purified by silica gel column chromatography using hexanes/EtOAc (0% to 25% EtOAc gradient) to give 58 (0.093 g, 0.208 mmol, 83% yield) as a clear colorless oil.

TLC: R_{f}=0.29 (hexanes/EtOAc, 30% EtOAc, UV). ¹H NMR: (500 MHz, CDCl₃) δ 8.16 (dd, J=8.3, 1.4 Hz, 2H), 7.75 (d, J=9.0 Hz, 2H), 7.56-7.51 (m, 1H), 7.45-7.41 (m, 2H), 7.11 (s, 1H), 7.02 (d, J=9.0 Hz, 2H), 5.06 (d, J=12.9 Hz, 1H), 4.99 (d, J=12.9 Hz, 1H), 3.90 (s, 3H), 2.21 (s, 3H) ppm. ¹³C NMR: (126 MHz, CDCl₃) δ 174.56, 173.72, 164.44, 161.50, 158.77, 135.49, 132.46, 130.89, 129.46, 128.15, 126.41, 117.93, 114.64, 62.34, 55.90, 13.87 ppm. HRMS: Calc'd for C₂₀H₁₉ClN₃O₃S₂ [M+H⁺] 448.0551; found: 448.0542.

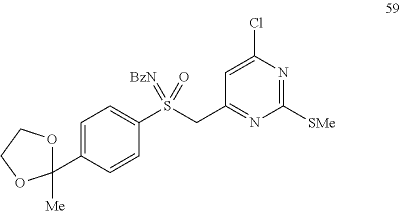

GP-4 was used (−78° C. for 1 hour, warmed to room temperature then stirred for 2 hours). Purified by silica gel column chromatography using hexanes/EtOAc (0% to 25% EtOAc gradient) to give 59 (111 mg, 0.220 mmol, 88% yield) clear colorless oil that solidified upon standing.

TLC: R_{f}=0.50 (hexanes/EtOAc, 40% EtOAc, UV). ¹H NMR: (500 MHz, CDCl₃) δ 8.15 (dd, J=8.3, 1.4 Hz, 2H), 7.83-7.76 (m, 2H), 7.70-7.65 (m, 2H), 7.57-7.50 (m, 1H), 7.46-7.41 (m, 2H), 7.11 (s, 1H), 5.05 (d, J=13.0 Hz, 1H), 5.01 (d, J=13.0 Hz, 1H), 4.09-4.05 (m, 2H), 3.78-3.74 (m, 2H), 2.20 (s, 3H), 1.65 (s, 3H) ppm. ¹³C NMR: (126 MHz, CDCl₃) δ 174.54, 173.68, 161.57, 158.48, 150.63, 135.30, 134.88, 132.59, 129.49, 128.73, 128.20, 126.45, 117.98, 108.13, 64.75, 62.19, 27.50, 13.95 ppm. HRMS: Calc'd for C₂₃H₂₂ClN₃O₄S₂Na [M+Na⁺] 526.0632; found: 526.0636.

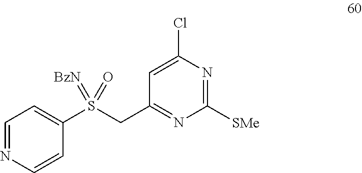

GP-4 was used (−78° C. for 30 minutes, warmed to room temperature then stirred for 1 hour). Purified by silica gel column chromatography using hexanes/EtOAc (0% to 40% EtOAc gradient) to give 60 (0.075 g, 0.179 mmol, 72% yield) as a light-yellow oil.

TLC: R_{f}=0.26 (hexanes/EtOAc, 40% EtOAc, UV). ¹H NMR: (500 MHz, CDCl₃) δ 8.95-8.88 (m, 2H), 8.13 (dd, J=8.4, 1.3 Hz, 2H), 7.75-7.70 (m, 2H), 7.58-7.54 (m, 1H), 7.45 (ddd, J=7.8, 7.0, 1.2 Hz, 2H), 7.20 (s, 1H), 5.02 (s, 2H), 2.20 (s, 3H) ppm. ¹³C NMR: (126 MHz, CDCl₃) δ 174.40, 174.15, 161.96, 157.53, 151.18, 144.96, 134.63, 132.94, 129.55, 128.30, 121.66, 118.07, 61.55, 13.87 ppm. HRMS: Calc'd for C₁₈H₁₆ClN₄O₂S₂ [M+H⁺]419.0398; found: 419.0399.

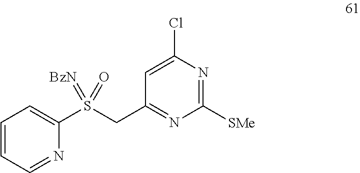

GP-4 was used (−78° C. for 30 minutes, warmed to 0° C. then stirred for 1 hour). Purified by silica gel column chromatography using hexanes/EtOAc (0% to 30% EtOAc gradient) to give 61 (0.086 g, 0.205 mmol, 82% yield) as an amorphous solid.

TLC: R_{f}=0.43 (hexanes/EtOAc, 40% EtOAc, UV). ¹H NMR: (500 MHz, CDCl₃) δ 8.80-8.77 (m, 1H), 8.18 (dt, J=7.9, 1.0 Hz, 1H), 8.12-8.08 (m, 2H), 7.99 (td, J=7.8, 1.7 Hz, 1H), 7.62-7.57 (m, 1H), 7.54-7.48 (m, 1H), 7.40 (t, J=7.7 Hz, 2H), 7.15 (s, 1H), 5.26 (d, J=13.4 Hz, 1H), 5.19 (d, J=13.4 Hz, 1H), 2.19 (s, 3H) ppm. ¹³C NMR: (126 MHz, CDCl₃) δ 174.26, 173.86, 161.57, 158.17, 155.11, 150.29, 138.22, 134.94, 132.52, 129.54, 128.11, 127.65, 124.52, 118.14, 57.78, 13.95 ppm. HRMS: Calc'd for C₁₈H₁₆ClN₄O₂S₂ [M+H⁺] 419.0398; found: 419.0396.

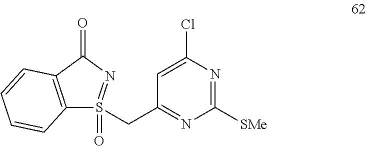 [SMILES: CSc1nc(Cl)cc(CS2(=O)=NC(=O)c3ccccc32)n1]

GP-5 was used (60° C. for 17 hours). Purified by silica gel column chromatography using hexanes/EtOAc (10% to 50% EtOAc gradient) to give 62 (0.069 g, 0.203 mmol, 81% yield) as a white-solid. Note: Solubility of sulfoximine proved problematic with GP-4.

TLC: R_{f}=0.33 (hexanes/EtOAc, 50% EtOAc, UV). ¹H NMR: (500 MHz, CDCl₃) δ 8.03-7.99 (m, 1H), 7.94 (dt, J=7.6, 0.8 Hz, 1H), 7.87 (td, J=7.5, 1.0 Hz, 1H), 7.80 (td, J=7.6, 1.1 Hz, 1H), 7.22 (s, 1H), 5.02 (d, J=14.0 Hz, 1H), 4.72 (d, J=13.9 Hz, 1H), 2.41 (s, 3H) ppm. ¹³C NMR: (126 MHz, CDCl₃) δ 174.46, 170.29, 162.43, 156.80, 139.10, 136.33, 136.01, 132.96, 125.71, 124.40, 118.01, 60.54, 14.16 ppm. HRMS: Calc'd for C₁₃H₁₀ClN₃O₂S₂Na [M+Na⁺] 361.9795; found: 361.9798.

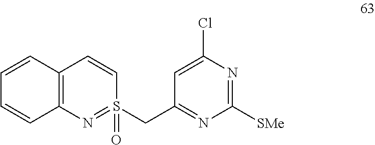 [SMILES: CSc1nc(Cl)cc(CS2(=O)=Nc3ccccc3C=C2)n1]

GP-4 was used (−78° C. for 1 hour, warmed to room temperature over 1.5 hours). Purified by silica gel column chromatography using hexanes/EtOAc (0% to 20% EtOAc gradient) to give 63 (0.057 g, 0.169 mmol, 67% yield) as a light-yellow oil that solidified upon standing. Note: Full consumption of starting materials was not achieved.

TLC: R_{f}=0.44 (hexanes/EtOAc, 30% EtOAc, UV). ¹H NMR: (500 MHz, CDCl₃) δ 7.63 (d, J=9.9 Hz, 1H), 7.42 (ddd, J=8.5, 7.1, 1.6 Hz, 1H), 7.27 (d, J=1.6 Hz, 2H), 7.25-7.19 (m, 1H), 7.13 (s, 1H), 6.98 (ddd, J=8.2, 7.1, 1.2 Hz, 1H), 6.51 (d, J=9.9 Hz, 1H), 4.72 (d, J=13.8 Hz, 1H), 4.68 (d, J=13.8 Hz, 1H), 2.55 (s, 3H) ppm. ¹³C NMR: (126 MHz, CDCl₃) δ 173.97, 161.82, 158.91, 145.50, 141.38, 132.46, 129.86, 123.64, 120.52, 117.81, 116.29, 106.95, 65.30, 14.36 ppm. HRMS: Calc'd for C₁₄H₁₃ClN₃OS₂ [M+H⁺]338.0183; found: 338.0187.

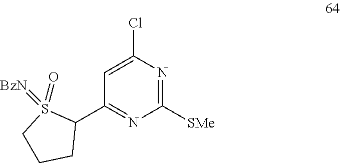

GP-4 was used (−78° C. for 1 hour then quenched). Purified by silica gel column chromatography using hexanes/EtOAc (0% to 20% EtOAc gradient) to give 64 (0.074 g, 0.194 mmol, 78% yield, d.r. 4:1 determined by ¹H NMR and LC-MS) as a clear colorless oil. Note: Fractions containing one of the diastereomers was contaminated with unreacted sulfoximine and were not included in the yield.

TLC: R_{f}=0.29 (hexanes/EtOAc, 30% EtOAc, UV) and R_{f}=0.18 (hexanes/EtOAc, 40% EtOAc, UV). Major diastereomer: ¹H NMR: (500 MHz, CDCl₃) δ 8.15 (dd, J=8.4, 1.4 Hz, 2H), 7.57-7.51 (m, 2H), 7.43 (dd, J=8.3, 7.0 Hz, 2H), 5.09 (t, J=7.7 Hz, 1H), 3.82-3.74 (m, 1H), 3.31 (ddd, J=13.0, 9.4, 7.3 Hz, 1H), 3.14-3.03 (m, 1H), 2.62-2.50 (m, 5H), 2.38-2.28 (m, 1H) ppm. ¹³C NMR: (126 MHz, CDCl₃) δ 175.18, 173.74, 161.77, 161.68, 134.97, 132.50, 129.42, 128.17, 118.60, 64.78, 52.86, 27.80, 21.16, 14.40 ppm. HRMS: Calc'd for C₁₆H₁₆ClN₃O₂S₂ [M+H⁺] 382.0445; found: 382.0446.

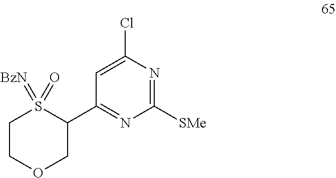

GP-4 was used (−78° C. for 1 hour, warmed to room temperature then stirred for 1 hour). Purified by silica gel column chromatography using hexanes/EtOAc (0% to 40% EtOAc gradient) to give 65 (0.068 g, 0.171 mmol, 68% yield, d.r. 1:1 determined by LC-MS) as a clear colorless oil. Note: Full consumption of starting materials was not achieved

TLC: R_{f}=0.42 (hexanes/EtOAc, 40% EtOAc, UV)¹H NMR: (500 MHz, CDCl₃) δ 7.99 (dd, J=8.3, 1.3 Hz, 2H), 7.54-7.47 (m, 1H), 7.39 (t, J=7.7 Hz, 2H), 7.35 (s, 1H), 4.65-4.45 (m, 4H), 4.34 (dt, J=13.1, 4.6 Hz, 1H), 4.07 (ddd, J=12.8, 10.1, 2.3 Hz, 1H), 3.56 (ddd, J=13.9, 10.1, 3.8 Hz, 1H), 2.57 (s, 3H) ppm. ¹³C NMR: (126 MHz, CDCl₃) δ 174.13, 174.00, 161.44, 158.31, 134.92, 132.56, 129.48, 128.12, 117.51, 69.28, 66.77, 65.94, 50.95, 14.36 ppm. HRMS: Calc'd for C₁₆H₁₇ClN₃O₃S₂ [M+H⁺] 398.0394; found: 398.0393.

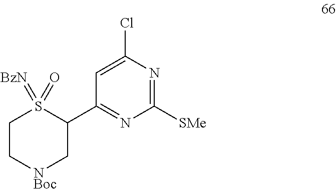

GP-4 was used (−78° C. for 1 hour, warmed to 0° C. then quenched). Purified by silica gel column chromatography using hexanes/EtOAc (0% to 25% EtOAc gradient) to give 66 (0.120 g, 0.241 mmol, 97% yield, d.r. 1.6:1 determined by LC-MS) as a clear colorless oil.

TLC: R_{f}=0.41 (hexanes/EtOAc, 30% EtOAc, UV). *NMR analysis of combined diastereomers. ¹H NMR: (500 MHz, CDCl₃) δ 8.11 (d, J=7.6 Hz, 2H), 7.53 (t, J=7.4 Hz, 1H), 7.42 (t, J=7.6 Hz, 2H), 7.11 (s, 1H), 5.20 (d, J=64.1 Hz, 1H), 4.32 (s, 2H), 4.22-3.87 (m, 3H), 3.84-3.75 (m, 1H), 2.52 (s, 3H), 1.41 (d, J=73.6 Hz, 9H) ppm. ¹³C NMR: (126 MHz, CDCl₃) δ 174.29, 161.66, 160.78, 153.42, 135.02, 132.63, 129.43, 128.19, 117.24, 115.74, 81.71, 62.51, 50.14, 46.81, 41.66, 28.17, 15.71 ppm. HRMS: Calc'd for C₂₁H₂₆ClN₄O₄S₂ [M+H⁺] 497.1079; found: 497.1074.

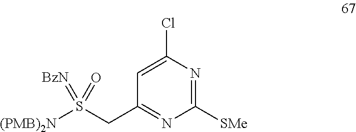

GP-4 was used (−78° C. for 1 hour, warmed to 0° C. then stirred for 1 hour). Purified by silica gel column chromatography using hexanes/EtOAc (0% to 20% EtOAc gradient) to give 67 (1.52 g, 2.55 mmol, 82% yield) as an amber oil.

TLC: R_{f}=0.31 (hexanes/EtOAc, 20% EtOAc, UV). ¹H NMR: (600 MHz, CDCl₃) δ 8.10 (dd, J=8.3, 1.4 Hz, 2H), 7.54-7.49 (m, 1H), 7.43-7.38 (m, 2H), 7.22-7.17 (m, 4H), 7.03 (s, 1H), 6.87-6.82 (m, 4H), 4.79 (d, J=13.4 Hz, 1H), 4.72 (d, J=13.4 Hz, 1H), 4.35 (s, 4H), 3.81 (s, 6H), 2.45 (s, 3H) ppm. ¹³C NMR: (151 MHz, CDCl₃) δ 173.83, 172.99, 161.51, 159.45, 159.05, 135.62, 132.36, 130.35, 129.46, 128.15, 127.00, 117.95, 114.07, 60.22, 55.30, 50.32, 14.16 ppm. HRMS: Calc'd for C₂₉H₃₀ClN₄O₄S₂Na [M+H⁺] 597.1392; found: 597.1387.

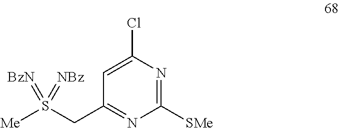

GP-4 was used (−78° C. for 1 hour, warmed to room temperature then stirred for 1 hour). Purified by silica gel column chromatography using hexanes/EtOAc (0% to 25% EtOAc gradient) to give 68 (0.074 g, 0.161 mmol, 65% yield) as a clear colorless oil that solidified upon standing.

GP-5 (50° C. for 14 hours) provided 68 (0.105 g, 0.229 mmol, 92% yield) as a clear colorless oil that solidified upon standing. Gram-scale using GP-5 provided 68 (2.18 g, 4.75 mmol, 88% yield)

TLC: R_{f}=0.47 (hexanes/EtOAc, 40% EtOAc, UV). ¹H NMR: (600 MHz, CDCl₃) δ 8.09 (dd, J=8.3, 1.4 Hz, 4H), 7.53-7.49 (m, 2H), 7.43-7.36 (m, 5H), 7.21 (s, 1H), 5.44 (s, 2H), 3.81 (s, 3H), 2.43 (s, 3H) ppm. ¹³C NMR: (151 MHz, CDCl₃) δ 174.70, 174.36, 162.05, 157.75, 134.97, 132.55, 129.43, 128.11, 118.74, 57.65, 38.61, 14.21 ppm. HRMS: Calc'd for C₂₁H₁₉ClN₄O₂S₂Na [M+Na⁺] 481.0530; found: 481.0534.

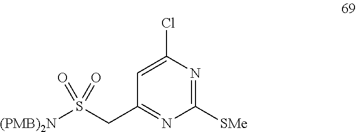

GP-4 was used with N,N-bis(4-methoxybenzyl)methanesulfonamide (−78° C. for 1.5 hours, warmed to room temperature then quenched). Purified by silica gel column chromatography using hexanes/EtOAc (0% to 30% EtOAc gradient) to give 69 (2.15 g, 4.35 mmol, 89% yield) as a golden oil.

TLC: R_{f}=0.31 (hexanes/EtOAc, 20% EtOAc, UV). ¹H NMR: (600 MHz, CDCl₃) δ 7.17 (d, J=8.6 Hz, 4H), 7.08 (s, 1H), 6.85 (d, J=8.6 Hz, 4H), 4.20 (s, 2H), 4.19 (s, 4H), 3.81 (s, 6H), 2.53 (s, 3H) ppm. ¹³C NMR: (151 MHz, CDCl₃) δ 173.88, 161.48, 159.72, 159.41, 130.17, 127.21, 117.31, 114.07, 59.94, 55.30, 50.00, 14.28 ppm. HRMS: Calc'd for C₂₂H₂₅ClN₃O₄S₂ [M+H⁺] 494.0970; found: 494.0970.

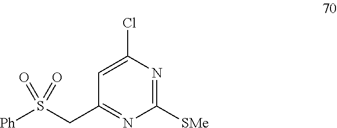 [SMILES: CSc1nc(Cl)cc(CS(=O)(=O)c2ccccc2)n1]

GP-4 was used with commercially available methyl phenyl sulfone (−78° C. for 1 hour, warmed to 0° C. then quenched). Purified by silica gel column chromatography using hexanes/EtOAc (0% to 30% EtOAc gradient) to give 70 (0.059 g, 0.181 mmol, 75% yield) as a white solid.

TLC: R_{f}=0.32 (hexanes/EtOAc, 30% EtOAc, UV). ¹H NMR: (600 MHz, CDCl₃) δ 7.72 (d, J=6.8 Hz, 2H), 7.67 (t, J=7.5 Hz, 1H), 7.54 (t, J=7.8 Hz, 2H), 7.14 (s, 1H), 4.39 (s, 2H), 2.24 (s, 3H) ppm. ¹³C NMR: (126 MHz, CDCl₃) δ 173.72, 161.18, 160.23, 142.15, 131.70, 129.28, 124.11, 117.19, 63.61, 14.17. HRMS: Calc'd for C₁₂H₁₁ClN₂O₂S₂ [M+H⁺]315.0023; found: 315.0021.

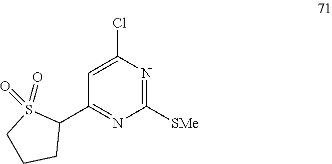 [SMILES: CSc1nc(Cl)cc(C2CCCS2(=O)=O)n1]

GP-4 was used with commercially available sulfolane (−78° C. for 20 minutes, warmed to 0° C. then quenched). Purified by silica gel column chromatography using hexanes/EtOAc (0% to 40% EtOAc gradient) to give 71 (602 mg, 2.16 mmol, 99% yield) as an off-white solid.

TLC: R_{f}=0.29 (hexanes/EtOAc, 40% EtOAc, UV). ¹H NMR: (500 MHz, CDCl₃) δ 7.05 (s, 1H), 4.17 (t, J=8.1 Hz, 1H), 3.27-3.20 (m, 1H), 3.20-3.12 (m, 1H), 2.80-2.68 (m, 1H), 2.59-2.51 (m, 4H), 2.49-2.40 (m, 1H), 2.30-2.19 (m, 1H). ¹³C NMR: (126 MHz, CDCl₃) δ 174.11, 162.33, 161.44, 116.84, 66.23, 51.07, 26.96, 20.13, 14.39 ppm. HRMS: Calc'd for C₉H₁₂ClN₂O₂S₂ [M+H⁺] 279.0023; found: 279.0025.

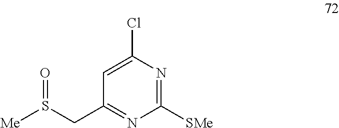 [SMILES: CSc1nc(Cl)cc(CS(C)=O)n1]

GP-4 was used with commercially available DMSO (−78° C. for 1 hour, warmed to room temperature then stirred for 30 minutes). Purified by silica gel column chromatography using hexanes/EtOAc (0% to 85% EtOAc gradient) to give 72 (49.0 mg, 0.207 mmol, 83% yield) as a light-yellow oil.

TLC: R_{f}=0.19 (hexanes/EtOAc, 80% EtOAc, UV). ¹H NMR: (500 MHz, CDCl₃) δ 7.05 (s, 1H), 4.04 (d, J=12.6 Hz, 1H), 3.89 (d, J=12.7 Hz, 1H), 2.64 (s, 3H), 2.56 (s, 3H) ppm. ¹³C NMR: (126 MHz, CDCl₃) δ 174.08, 161.65, 160.60, 117.26, 59.87, 38.48, 14.34 ppm. HRMS: Calc'd for C₇H₁₀ClN₂OS₂ [M+H⁺] 236.9918; found: 236.9921.

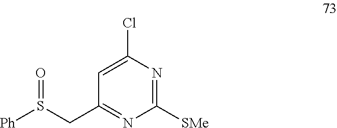 [SMILES: CSc1nc(Cl)cc(CS(=O)c2ccccc2)n1]

GP-4 was used with commercially available (methylsulfinyl)benzene (−78° C. for 1 hour, warmed to room temperature then stirred for 30 minutes). Purified by silica gel column chromatography using hexanes/EtOAc (0% to 85% EtOAc gradient) to give 73 (68.0 mg, 0.227 mmol, 91% yield) as a white solid.

TLC: R_{f}=0.64 (hexanes/EtOAc, 40% EtOAc, UV). ¹H NMR: (500 MHz, CDCl₃) δ 7.39-7.33 (m, 5H), 6.75 (s, 1H), 3.92 (d, J=12.4 Hz, 1H), 3.86 (d, J=12.4 Hz, 1H), 2.25 (s, 3H) ppm. ¹³C NMR: (126 MHz, CDCl₃) δ 173.71, 161.17, 160.23, 142.12, 131.71, 129.28, 124.12, 117.20, 108.60, 63.58, 14.17 ppm. HRMS: Calc'd for C₁₂H₁₂ClN₂OS₂ [M+H⁺]299.0074; found: 299.0077.

### Electrophile Scope:

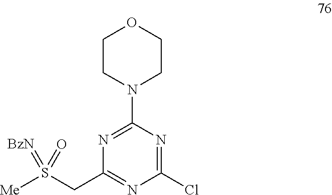

GP-4 was used with previously prepared

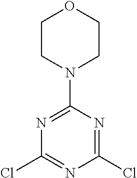 [SMILES: Clc1nc(Cl)nc(N2CCOCC2)n1]

(−78° C. for 1.5 hours then quenched). Purified by silica gel column chromatography using hexanes/EtOAc (0% to 40% EtOAc gradient) to give 76 (91.0 mg, 0.230 mmol, 92% yield) as a white solid.

TLC: R_{f}=0.15 (hexanes/EtOAc, 40% EtOAc, UV). ¹H NMR: (500 MHz, CDCl₃) δ 8.13-8.05 (m, 2H), 7.53-7.48 (m, 1H), 7.42-7.37 (m, 2H), 5.02 (d, J=14.1 Hz, 1H), 4.71 (d, J=14.0 Hz, 1H), 3.85-3.74 (m, 2H), 3.72-3.65 (m, 2H), 3.65-3.58 (m, 2H), 3.51 (ddd, J=12.1, 6.3, 3.4 Hz, 1H), 3.47 (s, 3H), 3.38-3.33 (m, 1H) ppm. ¹³C NMR: (126 MHz, CDCl₃) δ 173.93, 170.71, 168.23, 164.04, 135.27, 132.37, 129.30, 128.09, 66.27, 66.16, 60.58, 44.19, 44.00, 41.02 ppm. *NMR spectra slightly contaminated with sulfoximine starting material. HRMS: Calc'd for C₁₆H₁₉ClN₅O₃S [M+H⁺] 396.0892; found: 396.0888.

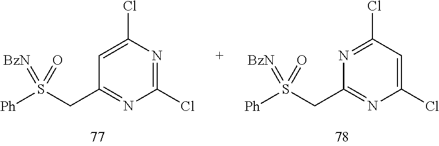

GP-4 was used with commercially available 2,4,6-trichloropyrimidine (−78° C. for 1 hour then quenched). Purified by silica gel column chromatography using hexanes/EtOAc (0% to 40% EtOAc gradient) to give 77 and 78 (79.0 mg, 0.194 mmol, 78% yield) as a light-yellow oil and mixture of regioisomers (1:2, determined by LC-MS). Note: Minor isomer (77) was contaminated with the major isomer. Characterization data was not obtained for 77.

TLC: R_{f}=0.32 (hexanes/EtOAc, 30% EtOAc, UV). ¹H NMR: (500 MHz, CDCl₃, major isomer) δ 8.19-8.14 (m, 2H), 7.92-7.86 (m, 2H), 7.72-7.67 (m, 1H), 7.60-7.55 (m, 2H), 7.55-7.50 (m, 1H), 7.45-7.40 (m, 2H), 7.28 (s, 1H), 5.31 (d, J=13.0 Hz, 1H), 5.13 (d, J=13.0 Hz, 1H) ppm. ¹³C NMR: (126 MHz, CDCl₃, major isomer) δ 174.18, 162.15, 160.34, 136.21, 135.50, 134.32, 132.31, 129.55, 129.38, 128.56, 128.06, 120.66, 63.42 ppm. HRMS: Calc'd for C₁₈H₁₃C₂₁N₃O₂SNa [M+Na⁺] 427.9998; found: 427.9995.

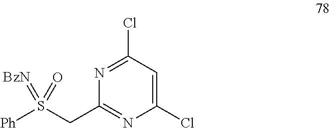

GP-4 was used with previously prepared

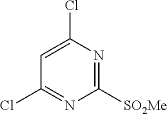 [SMILES: CS(=O)(=O)c1nc(Cl)cc(Cl)n1]

(−78° C. for 1 hour then quenched). Purified by silica gel column chromatography using hexanes/EtOAc (0% to 40% EtOAc gradient) to give 78 (94.0 mg, 0.231 mmol, 93% yield) as a clear colorless oil.

TLC: R_{f}=0.30 (hexanes/EtOAc, 30% EtOAc, UV). ¹H NMR: (500 MHz, CDCl₃) δ 8.18-8.14 (m, 2H), 7.92-7.86 (m, 2H), 7.71-7.67 (m, 1H), 7.61-7.53 (m, 2H), 7.54-7.49 (m, 1H), 7.42 (t, J=7.7 Hz, 2H), 7.27 (s, 1H), 5.30 (d, J=13.0 Hz, 1H), 5.13 (d, J=13.0 Hz, 1H) ppm. ¹³C NMR: (126 MHz, CDCl₃) δ 174.16, 162.14, 160.35, 136.23, 135.53, 134.31, 132.29, 129.54, 129.37, 128.56, 128.05, 120.66, 63.43 ppm. HRMS: Calc'd for C₁₈H₁₃Cl₂N₃O₂SNa [M+Na⁺] 427.9998; found: 427.9997.

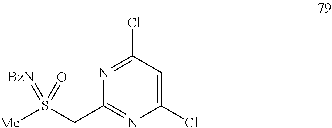

GP-4 was used with previously prepared

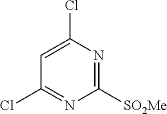 [SMILES: CS(=O)(=O)c1nc(Cl)cc(Cl)n1]

(−78° C. for 1 hour then quenched). Purified by silica gel column chromatography using hexanes/EtOAc (0% to 40% EtOAc gradient) to give 79 (77.0 mg, 0.223 mmol, 89% yield) as a clear colorless oil.

TLC: R_{f}=0.39 (hexanes/EtOAc, 40% EtOAc, UV). ¹H NMR: (500 MHz, CDCl₃) δ 8.07 (dd, J=8.4, 1.4 Hz, 2H), 7.53-7.47 (m, 1H), 7.42-7.36 (m, 3H), 5.16 (d, J=14.6 Hz, 1H), 4.97 (d, J=14.6 Hz, 1H), 3.56 (d, J=0.9 Hz, 3H) ppm. ¹³C NMR: (126 MHz, CDCl₃) δ 173.91, 162.64, 160.39, 135.19, 132.31, 129.37, 128.04, 121.15, 61.67, 40.59. HRMS: Calc'd for C₁₃H₁₂Cl₂N₃O₂S [M+H⁺] 344.0022; found: 344.0019.

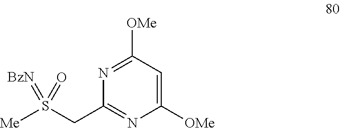

GP-4 was used with commercially available 2-Chloro-4,6-dimethoxypyrimidine (−78° C. for 3 hours then warmed to room temperature and stirred for 12 hours). Purified by silica gel column chromatography using hexanes/EtOAc (0% to 40% EtOAc gradient) to give 80 (55.0 mg, 0.165 mmol, 66% yield) as a clear colorless oil.

TLC: R_{f}=0.26 (hexanes/EtOAc, 40% EtOAc, UV). ¹H NMR: (500 MHz, CDCl₃) δ 8.09 (dd, J=8.4, 1.4 Hz, 2H), 7.51-7.46 (m, 1H), 7.38 (t, J=7.6 Hz, 2H), 6.00 (s, 1H), 5.13 (d, J=14.4 Hz, 1H), 4.80 (d, J=14.4 Hz, 1H), 3.87 (s, 6H), 3.52 (s, 3H) ppm. ¹³C NMR: (126 MHz, CDCl₃) δ 173.97, 171.58, 157.99, 135.58, 132.10, 129.29, 127.98, 89.81, 61.66, 54.37, 40.37 ppm. HRMS: Calc'd for C₁₅H₁₈N₃O₄S [M+H⁺] 336.1013; found: 336.1018.

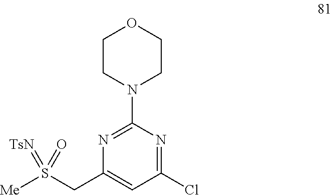

GP-5 was used with previously prepared

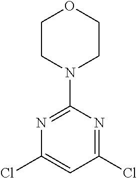 [SMILES: Clc1cc(Cl)nc(N2CCOCC2)n1]

(80° C. for 15 hours). Purified by silica gel column chromatography using hexanes/EtOAc (0% to 50% EtOAc gradient) to give 81 (91.0 mg, 0.205 mmol, 82% yield) as a white solid.

TLC: R_{f}=0.27 (hexanes/EtOAc, 50% EtOAc, UV). ¹H NMR: (500 MHz, CDCl₃) δ 7.89-7.81 (m, 2H), 7.31-7.26 (m, 2H), 6.82 (s, 1H), 4.73 (d, J=14.5 Hz, 1H), 4.69 (d, J=14.5 Hz, 1H), 3.81-3.77 (m, 4H), 3.76-3.72 (m, 4H), 3.28 (s, 3H), 2.41 (s, 3H) ppm. ¹³C NMR: (126 MHz, CDCl₃) δ 162.96, 161.12, 158.13, 143.18, 140.33, 129.39, 126.64, 111.52, 66.54, 62.44, 44.28, 42.00, 29.71, 21.57 ppm. HRMS: Calc'd for C₁₇H₂₁ClN₄O₄S₂Na [M+Na⁺] 467.0585; found: 467.0586.

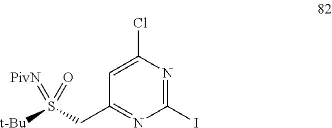

GP-4 was used with previously prepared

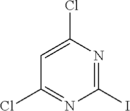

(−78° C. for 1.5 hours, removed from dry ice bath for 5 minutes then quenched). Purified by silica gel column chromatography using hexanes/EtOAc (0% to 20% EtOAc gradient) to give 82 (2.92 g, 6.38 mmol, 86% yield) as a yellow oil that solidified upon standing.

TLC: R_{f}=0.70 (hexanes/EtOAc, 40% EtOAc, UV). ¹H NMR: (500 MHz, CDCl₃) δ 7.85 (s, 1H), 5.04 (d, J=13.6 Hz, 1H), 4.70 (d, J=13.6 Hz, 1H), 1.49 (s, 9H), 1.19 (s, 9H) ppm. ¹³C NMR: (126 MHz, CDCl₃) δ 189.33, 161.49, 161.22, 126.54, 123.72, 64.00, 52.09, 41.85, 27.63, 23.35 ppm. Specific Rotation=+4.3 (c 1.13, CHCl₃). HRMS: Calc'd for C₁₄H₂₁ClIN₃O₂SNa [M+Na⁺] 479.9980; found: 479.9978.

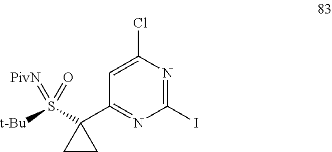

GP-4 was used with previously prepared

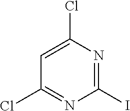

(−78° C. for 2.5 hours, warmed to 0° C. then quenched). Purified by silica gel column chromatography using hexanes/EtOAc (0% to 30% EtOAc gradient) to give 83 (1.48 g, 3.06 mmol, 75% yield) as a yellow oil. Note: 1.2 eq. of NaHMDS was used.

TLC: R_{f}=0.48 (hexanes/EtOAc, 20% EtOAc, UV)¹H NMR: (500 MHz, CDCl₃) δ 7.72 (s, 1H), 2.47 (ddd, J=10.6, 8.2, 6.0 Hz, 1H), 1.98 (ddd, J=9.6, 8.2, 5.7 Hz, 1H), 1.87 (ddd, J=10.6, 8.0, 5.7 Hz, 1H), 1.40 (s, 9H), 1.20 (s, 9H) ppm. ¹³C NMR: (126 MHz, CDCl₃) δ 188.03, 167.08, 161.00, 126.69, 123.60, 67.07, 42.24, 41.70, 27.73, 24.66, 17.04, 15.00 ppm. Specific rotation=−123.9 (c 1.00, CHCl₃). HRMS: Calc'd for C₁₆H₂₃ClIN₃O₂SNa [M+Na⁺] 506.0136; found: 506.0135.

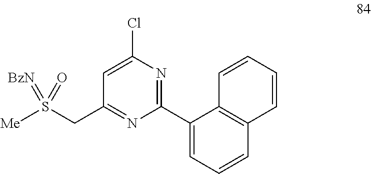

GP-4 was used with previously prepared

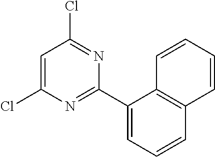 [SMILES: Clc1cc(Cl)nc(-c2cccc3ccccc23)n1]

(−78° C. for 2.5 hours, warmed to room temperature then quenched). Purified by silica gel column chromatography using hexanes/EtOAc (0% to 30% EtOAc gradient) to give 84 (92.0 mg, 0.211 mmol, 84% yield) as an amorphous solid.

TLC: R_{f}=0.21 (hexanes/EtOAc, 30% EtOAc, UV). ¹H NMR: (500 MHz, CDCl₃) δ 8.67-8.62 (m, 1H), 8.12-8.07 (m, 3H), 8.01 (d, J=8.2 Hz, 1H), 7.94-7.91 (m, 1H), 7.58-7.54 (m, 3H), 7.54 (s, 1H), 7.53-7.49 (m, 1H), 7.40 (t, J=7.7 Hz, 2H), 5.14 (d, J=14.5 Hz, 1H), 5.05 (d, J=14.4 Hz, 1H), 3.43 (s, 3H) ppm. ¹³C NMR: (126 MHz, CDCl₃) δ 174.43, 167.70, 162.53, 158.69, 134.97, 134.05, 133.42, 132.54, 131.87, 130.73, 130.32, 129.35, 128.75, 128.17, 127.42, 126.28, 125.30, 125.12, 120.92, 60.13, 41.04 ppm. HRMS: Calc'd for C₂₃H₁₁₈ClN₃O₂SNa [M+Na⁺] 458.0700; found: 458.0703.

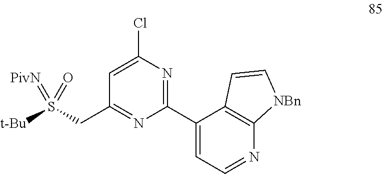

GP-4 was used with previously prepared

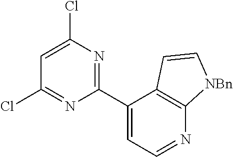 [SMILES: Clc1cc(Cl)nc(-c2ccnc3c2ccn3Cc2ccccc2)n1]

(−78° C. for 2 hours, warmed to room temperature then stirred for 1 hour). Purified by silica gel column chromatography using hexanes/EtOAc (0% to 30% EtOAc gradient) to give 85 (111 mg, 206 mmol, 83% yield) as a yellow oil. Note: Gram-scale reaction gave 85 (3.20 g, 5.95 mmol, 89% yield) as a yellow solid.

TLC: 0.67 (hexanes/EtOAc, 40% EtOAc, UV)¹H NMR: (500 MHz, CDCl₃) δ 8.49 (d, J=5.1 Hz, 1H), 8.11 (d, J=5.0 Hz, 1H), 7.78 (s, 1H), 7.44 (d, J=3.5 Hz, 1H), 7.36 (d, J=3.5 Hz, 1H), 7.33-7.26 (m, 3H), 7.24-7.21 (m, 2H), 5.58 (s, 2H), 5.26 (d, J=13.7 Hz, 1H), 4.99 (d, J=13.7 Hz, 1H), 1.52 (s, 9H), 1.23 (s, 9H) ppm. ¹³C NMR: (126 MHz, CDCl₃) δ 189.32, 165.01, 162.25, 160.39, 149.40, 142.79, 137.54, 134.80, 130.09, 128.76, 127.72, 127.46, 122.33, 118.81, 115.58, 102.12, 63.92, 53.29, 48.15, 41.94, 27.70, 23.56 ppm. Specific rotation=−5.46 (c 0.93, CHCl₃). HRMS: Calc'd for C₂₈H₃₃ClN₅O₂S [M+H⁺] 538.2038; found: 538.2034.

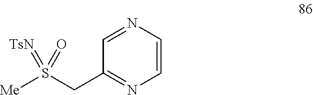

GP-5 was used with commercially available 2-chloropyrazine (80° C. for 18 hours). Purified by silica gel column chromatography using hexanes/EtOAc (0% to 40% EtOAc gradient) to give 86 (58.0 mg, 0.178 mmol, 71% yield) as a yellow oil.

TLC: R_{f}=0.16 (hexanes/EtOAc, 60% EtOAc, UV). ¹H NMR: (500 MHz, CDCl₃) δ 8.92 (d, J=1.5 Hz, 1H), 8.67 (d, J=2.4 Hz, 1H), 8.59 (dd, J=2.5, 1.4 Hz, 1H), 7.85 (d, J=8.3 Hz, 2H), 7.28 (d, J=8.1 Hz, 2H), 5.01 (s, 2H), 3.25 (s, 3H), 2.41 (s, 3H). ¹³C NMR: (126 MHz, CDCl₃) δ 147.67, 145.41, 144.53, 144.37, 143.18, 140.31, 129.39, 126.68, 60.94, 41.56, 21.57. HRMS: Calc'd for C₁₃H₁₆N₃O₃S₂[M+H⁺] 326.0628; found: 326.0625.

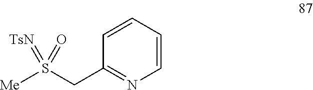

GP-5 was used with commercially available 2-fluoropyridine (80° C. for 17 hours). Purified by silica gel column chromatography using hexanes/EtOAc (0% to 70% EtOAc gradient) to give 87 (66.0 mg, 0.203 mmol, 81% yield) as a white solid.

TLC: R_{f}=0.28 (hexanes/EtOAc, 70% EtOAc, UV). ¹H NMR: (500 MHz, CDCl₃) δ 8.60 (d, J=4.8 Hz, 1H), 7.86 (d, J=8.4 Hz, 2H), 7.81 (td, J=7.7, 1.8 Hz, 1H), 7.71 (d, J=7.8 Hz, 1H), 7.36 (ddd, J=7.6, 4.9, 1.2 Hz, 1H), 7.27 (d, J=9.3 Hz, 2H), 5.05 (d, J=13.8 Hz, 1H), 4.90 (d, J=13.8 Hz, 1H), 3.19 (s, 3H), 2.40 (s, 3H) ppm. ¹³C NMR: (126 MHz, CDCl₃) δ 149.95, 148.30, 142.94, 140.62, 137.68, 129.31, 127.13, 126.66, 124.31, 63.53, 41.13, 21.55 ppm. HRMS: Calc'd for C₁₄H₁₆N₂O₃S₂Na [M+Na⁺] 347.0495; found: 347.0496.

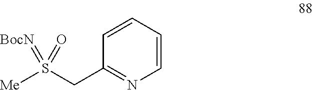

GP-5 was used with commercially available 2-fluoropyridine (80° C. for 15 hours). Purified by silica gel column chromatography using hexanes/EtOAc (0% to 70% EtOAc gradient) to give 88 (47.0 mg, 0.174 mmol, 70% yield) as a light-yellow oil.

TLC: R_{f}=0.3 (hexanes/EtOAc, 80% EtOAc, UV). ¹H NMR: (500 MHz, CDCl₃) δ 8.67-8.52 (m, 1H), 7.74 (td, J=7.7, 1.8 Hz, 1H), 7.51 (d, J=7.8 Hz, 1H), 7.32 (ddd, J=7.6, 4.9, 1.0 Hz, 1H), 5.02 (d, J=13.7 Hz, 1H), 4.89 (d, J=13.7 Hz, 1H), 3.13 (s, 3H), 1.49 (s, 9H) ppm. ¹³C NMR: (126 MHz, CDCl₃) δ 158.52, 149.94, 149.07, 137.42, 126.43, 124.01, 80.57, 60.55, 39.79, 28.15 ppm. HRMS: Calc'd for C₁₂H₁₈N₂O₃S [M+Na⁺] 293.0930; found: 293.0933.

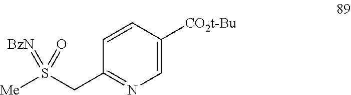

GP-4 was used with previously prepared

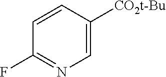 [SMILES: CC(C)(C)OC(=O)c1ccc(F)nc1]

(−78° C. to room temperature then stirred for 10 hours). Purified by silica gel column chromatography using hexanes/EtOAc (0% to 40% EtOAc gradient) to give 89 (67.0 mg, 0.179 mmol, 72% yield) as a clear colorless oil. Note: Claisen condensation side-product was observed.

TLC: R^{f}=0.12 (hexanes/EtOAc, 30% EtOAc, UV). ¹H NMR: (600 MHz, CDCl₃) δ 9.15 (d, J=2.2 Hz, 1H), 8.25 (dd, J=8.1, 2.2 Hz, 1H), 8.07 (dd, J=8.2, 1.4 Hz, 2H), 7.60 (d, J=8.1 Hz, 1H), 7.53-7.48 (m, 1H), 7.40 (t, J=7.7 Hz, 2H), 5.20 (d, J=13.5 Hz, 1H), 5.00 (d, J=13.5 Hz, 1H), 3.32 (s, 3H), 1.60 (s, 9H) ppm. ¹³C NMR: (126 MHz, CDCl₃) δ 174.44, 163.67, 152.21, 150.95, 138.28, 135.28, 132.27, 129.32, 128.05, 127.86, 126.00, 82.59, 61.11, 39.87, 28.13 ppm. HRMS: Calc'd for C₁₉H₂₃N₂O₄S [M+H⁺] 375.1373; found: 375.1373.

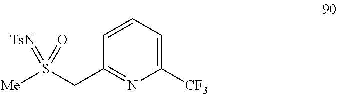

GP-5 was used with commercially available 2-chloro-6-(trifluoromethyl)pyridine (80° C. for 12 hours). Purified by silica gel column chromatography using hexanes/EtOAc (0% to 30% EtOAc gradient) to give 90 (22.0 mg, 0.056 mmol, 22% yield) as a light-yellow oil.

TLC: R_{f}=0.36 (hexanes/EtOAc, 40% EtOAc, UV). ¹H NMR: (500 MHz, CDCl₃) δ 8.04 (t, J=7.8 Hz, 1H), 7.97 (d, J=7.8 Hz, 1H), 7.85 (d, J=8.3 Hz, 2H), 7.76 (dd, J=7.8, 1.0 Hz, 1H), 7.28 (d, J=8.1 Hz, 2H), 5.20 (d, J=13.7 Hz, 1H), 4.99 (d, J=13.8 Hz, 1H), 3.21 (s, 3H), 2.41 (s, 3H) ppm. ¹³C NMR: (126 MHz, CDCl₃) δ 148.41 (q, J=35.2 Hz), 148.83, 148.55, 148.27, 147.99, 143.15, 140.36, 139.45, 129.87, 129.36, 126.66, 120.92 (t, J=2.8 Hz), 62.74, 41.27, 21.56 ppm. 19F NMR: (471 MHz, CDCl₃) δ −68.19 ppm. HRMS: Calc'd for C₁₅H₁₆F₃N₂O₃S₂ [M+H⁺] 393.0549; found: 393.0546.

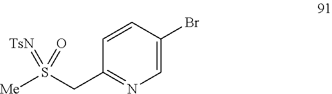

GP-5 was used with commercially available 2-fluoro-5-bromopyridine (80° C. for 15 hours). Purified by silica gel column chromatography using hexanes/EtOAc (0% to 40% EtOAc gradient) to give 91 (88.0 mg, 0.220 mmol, 88% yield) as a light-yellow oil.

TLC: R_{f}=0.49 (hexanes/EtOAc, 50% EtOAc, UV). ¹H NMR: (500 MHz, CDCl₃) δ 8.66 (d, J=2.4 Hz, 1H), 7.94 (dd, J=8.3, 2.4 Hz, 1H), 7.84 (d, J=8.3 Hz, 2H), 7.63 (d, J=8.2 Hz, 1H), 7.27 (d, J=8.2 Hz, 2H), 5.03 (d, J=14.8 Hz, 1H), 4.86 (d, J=13.8 Hz, 1H), 3.18 (s, 3H), 2.41 (s, 3H) ppm. ¹³C NMR: (126 MHz, CDCl₃) δ 151.16, 146.83, 143.06, 140.44, 140.34, 129.35, 128.27, 126.64, 122.06, 62.84, 41.17, 21.56 ppm. HRMS: Calc'd for C₁₄H₁₆BrN₂O₃S₂ [M+H⁺] 402.9780; found: 402.9778.

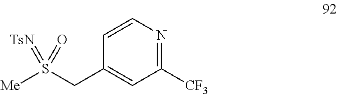

GP-5 was used with commercially available 2-chloro-4-fluoropyridine (80° C. for 3 hours). Purified by silica gel column chromatography using hexanes/EtOAc (0% to 40% EtOAc gradient) to give 92 (65.0 mg, 0.183 mmol, 73% yield) as an off-white solid.

TLC: R_{f}=0.33 (hexanes/EtOAc, 60% EtOAc, UV). ¹H NMR: (500 MHz, Chloroform-d) δ 8.48 (d, J=5.0 Hz, 1H), 7.82 (d, J=8.4 Hz, 2H), 7.47 (s, 1H), 7.40 (dd, J=5.1, 1.5 Hz, 1H), 7.29 (d, J=8.0 Hz, 2H), 4.81 (d, J=14.0 Hz, 1H), 4.74 (d, J=14.1 Hz, 1H), 3.14 (s, 3H), 2.42 (s, 3H). ¹³C NMR: (126 MHz, CDCl₃) δ 152.59, 150.64, 143.39, 140.07, 138.69, 129.45, 126.56, 126.25, 124.31, 60.39, 40.79, 21.58. HRMS: Calc'd for C₁₄H₁₆ClN₂O₃S₂ [M+H⁺] 359.0285; found: 359.0281.

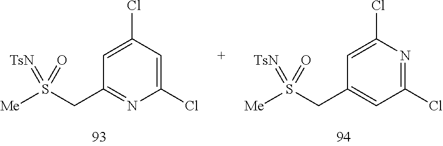

GP-5 was used with commercially available 2,4,6-trichloropyridine (80° C. for 2 hours). Purified by silica gel column chromatography using hexanes/EtOAc (0% to 40% EtOAc gradient) to give 93 (66.0 mg, 0.165 mmol, 66% yield) as an amorphous solid and 94 (10 mg, 0.025 mmol, 10% yield) as a white solid. Note: 93 eluted first (27% EtOAc) followed by 94 (40% EtOAc)

(93) TLC: R_{f}=0.44 (hexanes/EtOAc, 40% EtOAc, UV). ¹H NMR: (500 MHz, CDCl₃) δ 7.86-7.80 (m, 2H), 7.65 (d, J=1.6 Hz, 1H), 7.41 (d, J=1.6 Hz, 1H), 7.30-7.25 (m, 3H), 4.97 (d, J=14.0 Hz, 1H), 4.90 (d, J=13.9 Hz, 1H), 3.25 (s, 3H), 2.40 (s, 3H) ppm. ¹³C NMR: (126 MHz, CDCl₃) δ 152.05, 149.76, 147.42, 143.20, 140.28, 129.40, 126.63, 126.27, 125.02, 62.35, 41.59, 21.57 ppm. HRMS: Calc'd for C₁₄H₁₅Cl₂N₂O₃S₂[M+H⁺]392.9896; found: 392.9893.

(94) TLC: R_{f}=0.21 (hexanes/EtOAc, 40% EtOAc, UV). ¹H NMR: (500 MHz, CDCl³) δ 7.84-7.78 (m, 2H), 7.42 (s, 2H), 7.32-7.27 (m, 2H), 4.85 (d, J=13.9 Hz, 1H), 4.71 (d, J=13.9 Hz, 1H), 3.21 (s, 3H), 2.42 (s, 3H) ppm. ¹³C NMR: (126 MHz, CDCl₃) δ 151.59, 143.56, 141.00, 139.90, 129.52, 126.52, 124.82, 59.75, 41.17, 21.59. HRMS: Calc'd for C₁₄H₁₅Cl₂N₂O₃S₂[M+H⁺] 392.9896; found: 392.9897.

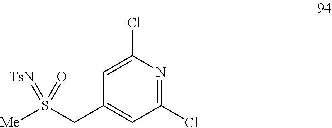

GP-5 was used with previously prepared

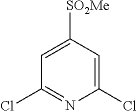 [SMILES: CS(=O)(=O)c1cc(Cl)nc(Cl)c1]

(80° C. for 15 hours). Purified by silica gel column chromatography using hexanes/EtOAc (0% to 50% EtOAc gradient) to give 94 (78.0 mg, 0.198 mmol, 79% yield) as a white solid.

TLC: R_{f}=0.21 (hexanes/EtOAc, 40% EtOAc, UV). ¹H NMR: (500 MHz, CDCl₃) δ 7.84-7.77 (m, 2H), 7.43 (s, 2H), 7.32-7.26 (m, 2H), 4.86 (d, J=14.0 Hz, 1H), 4.72 (d, J=13.9 Hz, 1H), 3.21 (s, 3H), 2.42 (s, 3H) ppm. ¹³C NMR: (126 MHz, CDCl₃) δ 151.55, 143.56, 141.02, 139.88, 129.52, 126.49, 124.84, 59.69, 41.15, 21.59 ppm. HRMS: Calc'd for C₁₄H₁₅Cl₂N₂O₃S₂[M+H⁺] 392.9896; found: 392.9898.

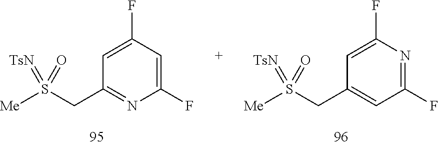

GP-5 was used with commercially available 2,4,6-trifluoropyridine (80° C. for 18 hours). Purified by silica gel column chromatography using hexanes/EtOAc (0% to 50% EtOAc gradient) to give 95 (45.0 mg, 0.124 mmol, 50% yield) as a clear colorless oil and 96 (24 mg, 0.067 mmol, 27% yield) as a clear colorless oil. Note: 95 eluted first (25% EtOAc) followed by 96 (38% EtOAc).

(95) TLC: R_{f}=0.75 (hexanes/EtOAc, 60% EtOAc, UV). ¹H NMR: (500 MHz, CDCl₃) δ 7.88-7.82 (m, 2H), 7.41 (dd, J=7.6, 1.8 Hz, 1H), 7.31-7.27 (m, 2H), 6.74 (dt, J=7.8, 1.9 Hz, 1H), 4.99 (d, J=13.9 Hz, 1H), 4.87 (d, J=13.9 Hz, 1H), 3.25 (s, 3H), 2.41 (s, 3H) ppm. ¹³C NMR: (126 MHz, CDCl₃) δ 171.76 (dd, J=267.0, 12.3 Hz), 164.26 (dd, J=242.5, 13.6 Hz), 148.88 (dd, J=16.8, 10.0 Hz), 143.23, 140.27, 129.40, 126.67, 113.91 (dd, J=20.0, 5.0 Hz), 98.87 (dd, J=40.6, 22.0 Hz), 62.39 (d, J=3.2 Hz), 41.73, 21.56 ppm. ¹⁹F NMR: (471 MHz, CDCl₃) δ −62.06 (d, J=22.5 Hz), −92.75 (d, J=23.4 Hz) ppm. HRMS: Calc'd for C₁₄H₁₅F₂N₂O₃S₂ [M+H⁺] 361.0487; found: 361.0484.

(96) TLC: R_{f}=0.5 (hexanes/EtOAc, 60% EtOAc, UV). ¹H NMR: (500 MHz, CDCl₃) δ 7.82 (d, J=8.4 Hz, 2H), 7.29 (d, J=7.9 Hz, 2H), 7.02 (s, 2H), 4.93 (d, J=13.9 Hz, 1H), 4.84 (d, J=14.0 Hz, 1H), 3.20 (s, 3H), 2.42 (s, 3H) ppm. ¹³C NMR: (126 MHz, CDCl₃) δ 162.01 (dd, J=249.1, 15.7 Hz), 145.27 (t, J=7.9 Hz), 143.57, 139.93, 129.50, 126.55, 108.58 (dd, J=16.1, 12.7 Hz), 60.08 (t, J=3.2 Hz), 41.03, 21.58 ppm. ¹⁹F NMR: (471 MHz, CDCl₃) δ −65.36 ppm. HRMS: Calc'd for C₁₄H₁₅F₂N₂O₃S₂ [M+H⁺] 361.0487; found: 361.0486.

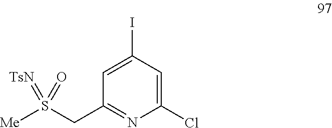

GP-5 was used with commercially available 2,6-dichloro-4-iodopyridine (80° C. for 15 hours). Purified by silica gel column chromatography using hexanes/EtOAc (0% to 50% EtOAc gradient) to give 97 (72.0 mg, 0.154 mmol, 62% yield) as a light-yellow oil. Note: regioselectivity for the 2-position was determined to be 19:1 by HPLC.

TLC: R_{f}=0.64 (hexanes/EtOAc, 50% EtOAc, UV). ¹H NMR: (500 MHz, CDCl₃) δ 7.99 (d, J=1.2 Hz, 1H), 7.87-7.83 (m, 2H), 7.80 (d, J=1.2 Hz, 1H), 7.31-7.27 (m, 2H), 4.92 (d, J=14.0 Hz, 1H), 4.87 (d, J=13.9 Hz, 1H), 3.24 (s, 3H), 2.42 (s, 3H) ppm. ¹³C NMR: (126 MHz, CDCl₃) δ 151.42, 148.99, 143.19, 140.27, 134.68, 133.70, 129.41, 126.65, 108.23, 62.12, 41.55, 21.58 ppm. HRMS: Calc'd for C₁₄H₁₄ClIN₂O₃S₂Na [M+Na⁺]506.9071; found: 506.9068.

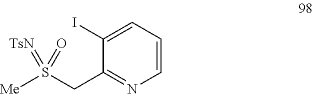

GP-5 was used with commercially available 2-chloro-3-iodopyridine (70° C. for 8 hours). Purified by silica gel column chromatography using hexanes/EtOAc (0% to 50% EtOAc gradient) to give 98 (49.0 mg, 0.109 mmol, 44% yield) as a clear colorless oil that solidified upon standing.

TLC: R_{f}=0.14 (hexanes/EtOAc, 4% EtOAc, UV). ¹H NMR: (500 MHz, CDCl₃) δ 8.56 (dd, J=4.6, 1.5 Hz, 1H), 8.21 (dd, J=8.1, 1.5 Hz, 1H), 7.91-7.87 (m, 2H), 7.29-7.25 (m, 2H), 7.04 (dd, J=8.1, 4.6 Hz, 1H), 5.62 (d, J=14.4 Hz, 1H), 4.88 (d, J=14.4 Hz, 1H), 3.40 (s, 3H), 2.40 (s, 3H) ppm. ¹³C NMR: (126 MHz, CDCl₃) δ 150.73, 148.91, 148.22, 142.87, 140.64, 129.29, 126.75, 125.08, 98.61, 64.95, 42.15, 21.56. ppm HRMS: Calc'd for C₁₄H₁₅IN₂O₃S₂Na [M+Na⁺] 472.9461; found: 472.9463.

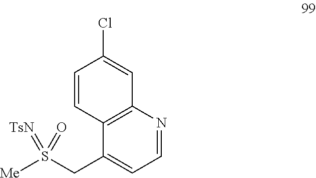

GP-5 was used with commercially available 4,7-dichloroquinoline (80° C. for 3 hours). Purified by silica gel column chromatography using hexanes/EtOAc (0% to 60% EtOAc gradient) to give 99 (75.0 mg, 0.183 mmol, 73% yield) as an off-white amorphous solid.

TLC: R_{f}=0.31 (DCM/MeOH, 5% MeOH, UV). ¹H NMR: (500 MHz, CDCl₃) δ 8.94 (d, J=4.4 Hz, 1H), 8.15 (d, J=2.0 Hz, 1H), 8.13 (d, J=9.2 Hz, 1H), 7.80 (d, J=8.2 Hz, 2H), 7.61-7.57 (m, 2H), 7.24 (d, J=8.1 Hz, 2H), 5.57 (d, J=14.5 Hz, 1H), 5.15 (d, J=14.3 Hz, 1H), 3.15 (s, 3H), 2.39 (s, 3H) ppm. ¹³C NMR: (126 MHz, CDCl₃) δ 150.98, 149.24, 143.33, 140.20, 136.29, 133.16, 129.45, 129.36, 129.05, 126.50, 125.53, 125.25, 124.88, 58.00, 40.90, 21.56 ppm. HRMS: Calc'd for C₁₈H₁₇N₂O₃S₂Na [M+Na⁺] 431.0261; found: 431.0266.

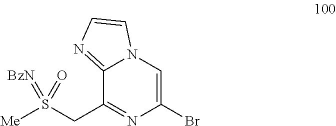

GP-4 with commercially available 6,8-dibromoimidazo[1,2-a]pyrazine (−78° C. for 1 hour, warmed to room temperature over 1.5 hours). Purified by silica gel column chromatography using hexanes/EtOAc (0% to 60% EtOAc gradient) to give 100 (83.0 mg, 0.211 mmol, 84% yield) as a light-yellow oil.

TLC: R_{f}=0.22 (hexanes/EtOAc, 60% EtOAc, UV). ¹H NMR: (500 MHz, CDCl₃) δ 8.31 (s, 1H), 8.09-8.03 (m, 2H), 7.89 (d, J=1.1 Hz, 1H), 7.76 (d, J=1.1 Hz, 1H), 7.51-7.47 (m, 1H), 7.38 (t, J=7.8 Hz, 2H), 5.50 (d, J=14.7 Hz, 1H), 5.39 (d, J=13.7 Hz, 1H), 3.61 (s, 3H) ppm. ¹³C NMR: (126 MHz, CDCl₃) δ 174.01, 142.36, 139.95, 137.43, 135.33, 132.19, 129.42, 127.99, 122.14, 120.01, 114.88, 56.65, 40.94 ppm. HRMS: Calc'd for C₁₅H₁₃BrN₄O₂SNa [M+Na⁺] 414.9835; found: 414.9836.

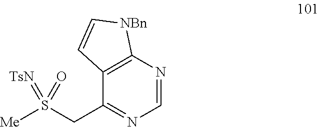

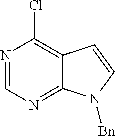 [SMILES: Clc1ncnc2c1ccn2Cc1ccccc1]

GP-5 was used with previously prepared (80° C. for 3 hours). Purified by silica gel column chromatography using hexanes/EtOAc (0% to 50% EtOAc gradient) to give 101 (89.7 mg, 0.197 mmol, 79% yield) as a clear colorless oil.

TLC: R_{f}=0.42 (hexanes/EtOAc, 60% EtOAc, UV). ¹H NMR: (500 MHz, CDCl₃) δ 8.88 (s, 1H), 7.87-7.81 (m, 2H), 7.35-7.29 (m, 4H), 7.26-7.23 (m, 4H), 6.90 (d, J=3.7 Hz, 1H), 5.51-5.43 (m, 2H), 5.42-5.37 (m, 1H), 5.12 (d, J=13.8 Hz, 1H), 3.35 (s, 3H), 2.39 (s, 3H) ppm. ¹³C NMR: (126 MHz, CDCl₃) δ 151.53, 151.16, 147.53, 142.98, 140.57, 136.20, 130.47, 129.32, 128.99, 128.24, 127.80, 126.66, 119.94, 100.25, 61.10, 48.24, 41.95, 21.55 ppm. *NMR spectra contaminated with unreacted sulfoximine starting material. HRMS: Calc'd for C₂₂H₂₃N₄O₃S₂[M+H⁺] 455.1206; found: 455.1199.

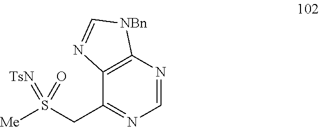

GP-5 was used with previously prepared

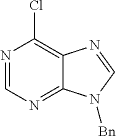 [SMILES: Clc1ncnc2c1ncn2Cc1ccccc1]

(60° C. for 24 hours) Purified using silica gel column chromatography hexanes/EtOAc (0% to 60% EtOAc gradient) to give 102 (89.0 mg, 0.195 mmol, 78% yield) as a yellow oil.

TLC: R_{f}=0.33 (DCM/MeOH, 5% MeOH, UV). ¹H NMR: (500 MHz, CDCl₃) δ 9.01 (s, 1H), 8.18 (s, 1H), 7.85-7.82 (m, 2H), 7.39-7.31 (m, 5H), 7.24-7.20 (m, 2H), 5.54 (d, J=14.0 Hz, 1H), 5.45 (d, J=2.8 Hz, 2H), 5.17 (d, J=14.0 Hz, 1H), 3.52 (s, 3H), 2.37 (s, 3H) ppm. ¹³C NMR: (126 MHz, CDCl₃) δ 152.59, 152.29, 147.11, 146.15, 142.89, 134.59, 134.01, 129.34, 129.28, 128.87, 128.14, 126.69, 126.61, 58.18, 47.70, 42.99, 21.53 ppm. *NMR spectra contaminated with unreacted sulfoximine starting material. HRMS: Calc'd for C₂₁H₂₂N₅O₃S₂ [M+H⁺] 456.1159; found: 456.1153.

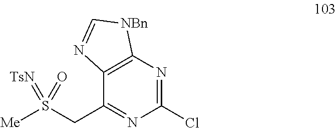

GP-5 was used with previously prepared

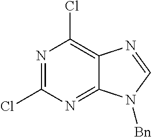 [SMILES: Clc1nc(Cl)c2ncn(Cc3ccccc3)c2n1]

(80° C. for 3 hours). Purified using silica gel column chromatography hexanes/EtOAc (0% to 60% EtOAc gradient) to give 103 (91.0 mg, 0.188 mmol, 74% yield) as a yellow amorphous solid.

TLC: R_{f}=0.24 (hexanes/EtOAc, 60% EtOAc, UV). ¹H NMR: (500 MHz, CDCl₃) δ 8.10 (s, 1H), 7.86-7.81 (m, 2H), 7.42-7.36 (m, 3H), 7.35-7.31 (m, 2H), 7.25-7.22 (m, 2H), 5.52 (d, J=14.1 Hz, 1H), 5.43 (d, J=14.9 Hz, 1H), 5.39 (d, J=15.0 Hz, 1H), 5.09 (d, J=14.1 Hz, 1H), 3.55 (s, 3H), 2.39 (s, 3H) ppm. ¹³C NMR: (126 MHz, CDCl₃) δ 154.19, 153.99, 149.09, 146.71, 143.01, 140.38, 133.95, 133.24, 129.41, 129.32, 129.11, 128.27, 126.70, 57.80, 47.87, 43.13, 21.54 ppm. HRMS: Calc'd for C₂₁H₂₁ClN₅O₃S₂ [M+H⁺]490.0769; found: 490.0767.

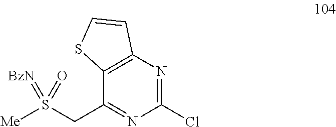

GP-4 was used with commercially available 2,4-dichlorothieno[3,2-d]pyrimidine (−78° C. for 1 hour, warmed to room temperature over 1.5 hours then quenched). Purified by silica gel column chromatography using hexanes/EtOAc (0% to 40% EtOAc gradient) to give 104 (77.0 mg, 0.211 mmol, 84% yield) as a light-yellow oil.

TLC: R_{f}=0.54 (hexanes/EtOAc, 30% EtOAc, UV). ¹H NMR: (500 MHz, CDCl₃) δ 8.19 (d, J=5.5 Hz, 1H), 8.06 (dd, J=8.3, 1.4 Hz, 2H), 7.55 (d, J=5.5 Hz, 1H), 7.53-7.48 (m, 1H), 7.41-7.36 (m, 2H), 5.39 (d, J=14.6 Hz, 1H), 5.16 (d, J=14.4 Hz, 1H), 3.52 (s, 3H) ppm. ¹³C NMR: (126 MHz, CDCl₃) δ 174.53, 164.38, 157.15, 154.06, 140.11, 134.97, 132.51, 131.60, 129.42, 128.11, 123.98, 59.53, 40.64 ppm. HRMS: Calc'd for C₁₅H₁₂ClN₃O₂S₂ [M+Na⁺] 387.9952; found: 387.9954.

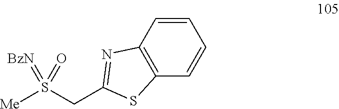

GP-5 was used with commercially available 2-chlorobenzo[d]thiazole was used (60° C. for 28 hours). Purified by silica gel column chromatography using hexanes/EtOAc (0% to 40% EtOAc gradient) to give 105 (58.0 mg, 0.176 mmol, 71% yield) as a yellow oil.

TLC: R_{f}=0.29 (hexanes/EtOAc, 40% EtOAc, UV). ¹H NMR: (500 MHz, CDCl₃) δ 8.13 (dd, J=8.4, 1.4 Hz, 2H), 8.07 (dt, J=8.3, 0.9 Hz, 1H), 7.89 (dt, J=8.0, 1.0 Hz, 1H), 7.57-7.49 (m, 2H), 7.45 (ddd, J=8.3, 7.2, 1.2 Hz, 1H), 7.41 (dd, J=8.2, 7.0 Hz, 2H), 5.54 (d, J=15.1 Hz, 1H), 5.24 (d, J=14.9 Hz, 1H), 3.40 (s, 3H) ppm. ¹³C NMR: (126 MHz, CDCl₃) δ 174.55, 156.63, 152.86, 136.39, 135.15, 132.40, 129.47, 128.09, 126.73, 126.26, 123.57, 121.90, 56.98, 39.63 ppm. HRMS: Calc'd for C₁₆H₁₅N₂O₂S₂[M+H⁺] 331.0569; found: 331.0574.

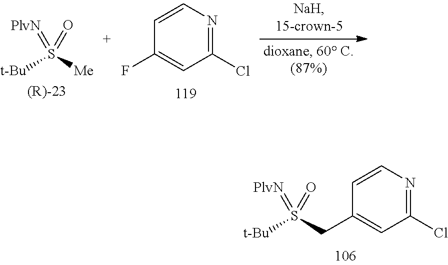

In a septum capped 40 mL vial equipped with a stir bar and argon balloon was added (R)-23 (0.440 g, 2.01 mmol, 1 eq.), 119 (0.263 g, 2.01 mmol, 1 eq.) and dioxane (16 mL). 15-Crown-5 ether (0.477 mL, 2.41 mmol, 1.2 eq.) was added followed by NaH (0.176 g, 4.41 mmol, 2.2 eq., 60% wt), stirred at room temperature for 3 minutes, then heated to 60° C. for 22 hours. The reaction was quenched with saturated aqueous NH₄Cl (15 mL), aqueous layer extracted with EtOAc (4×20 mL), combined organic layers dried over Na₂SO₄, filtered and concentrated. Purification using silica gel column chromatography using hexanes/EtOAc (0% to 20% EtOAc gradient) provided 106 (0.577 g, 1.74 mmol, 87% yield) as a white solid.

TLC: 0.24 (hexanes/EtOAc, 40% EtOAc, UV) ¹H NMR: (500 MHz, CDCl₃) δ 8.40 (dd, J=5.1, 0.7 Hz, 1H), 7.46 (d, J=0.8 Hz, 1H), 7.39 (dd, J=5.2, 1.5 Hz, 1H), 5.07 (d, J=13.5 Hz, 1H), 4.49 (d, J=13.5 Hz, 1H), 1.48 (s, 9H), 1.20-1.16 (m, 9H) ppm. ¹³C NMR: (126 MHz, CDCl₃) δ 188.01, 151.96, 149.86, 140.78, 126.43, 124.71, 64.36, 50.10, 41.90, 27.68, 23.86 ppm. Specific rotation=+23.1 (c 1.00, CHCl₃) HRMS: Calc'd for C₁₅H₂₄ClN₂O₂S [M+H⁺] 331.1242; found: 331.1242.

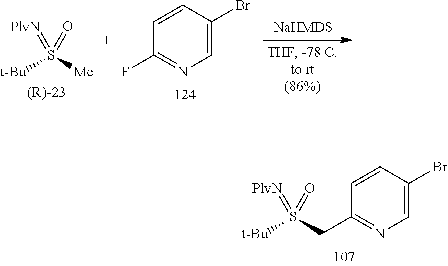

In a septum capped 25 mL round-bottomed flask equipped with a stir bar and argon balloon was added (R)-23 (250 mg, 1.14 mmol, 1.1 eq.) and THF (8 mL) then cooled to −78° C. NaHMDS (1.07 mL, 1.14 mmol, 2M in THF, 1.1 eq.) was added dropwise then stirred at −78° C. for 30 minutes. A solution of 124 (182 mg, 1.04 mmol, 1 eq.) in THF (2 mL) was added dropwise and continued stirring at −78° C. for 45 minutes. Another portion of NaHMDS (1.07 mml, 1.14 mmol, 2M in THF, 1.1 eq.) was added and the resulting reaction mixture stirred at −78° C. for 2 hours before warming to room temperature where it stirred for another 12 hours. Quenched with saturated aqueous NH₄Cl (15 mL), aqueous layer extracted with EtOAc (4×25 mL), combined organic layers dried over Na₂SO₄, filtered and concentrated. Further purification by silica gel column chromatography using hexanes/EtOAc (0% to 40% EtOAc gradient) provided 107 (335 mg, 0.893 mmol, 86% yield) as a light-yellow oil. Note: When method A was used without modifications, 73% yield was obtained.

TLC: 0.6 (hexanes/EtOAc, 40% EtOAc, UV)¹H NMR: (500 MHz, CDCl₃) δ 8.59 (dd, J=2.4, 0.6 Hz, 1H), 7.83 (dd, J=8.4, 2.4 Hz, 1H), 7.66 (d, J=8.4 Hz, 1H), 5.15 (d, J=13.9 Hz, 1H), 4.88 (d, J=13.9 Hz, 1H), 1.43 (s, 9H), 1.19 (s, 9H) ppm. ¹³C NMR: (126 MHz, CDCl₃) δ 189.09, 150.33, 148.79, 139.39, 128.41, 120.98, 63.28, 53.70, 41.80, 27.76, 23.44 ppm. Specific rotation=+67.6 (c 1.00, CHCl₃) HRMS: Calc'd for C₁₅H₂₃BrN₂O₂SNa [M+Na⁺] 397.0056; found: 397.0059.

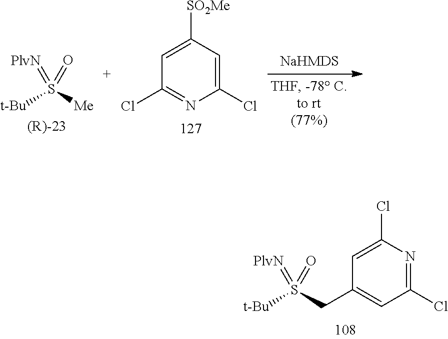

In a septum capped 100 mL round-bottomed flask equipped with a stir bar and argon balloon was added (R)-23 (615 mg, 2.80 mmol, 1.7 eq.), 127 (373 mg, 1.65 mmol, 1 eq.) and THF (28 mL) then cooled to −78° C. NaHMDS (1.81 mL, 3.63 mmol, 2M in THF, 2.2 eq.) was added dropwise over 30 minutes. The reaction mixture stirred at −78° C. for 2 hours before warming to room temperature where it stirred for 12 hours. Quenched with saturated aqueous NH₄Cl (20 mL), extracted with EtOAc (4×30 mL), combined organic layers dried over Na₂SO₄, filtered and concentrated. Further purification by silica gel column chromatography using hexanes/EtOAc (0% to 40% EtOAc gradient) provided 108 (466 mg, 1.28 mmol, 77% yield) as a white solid. Note: Slow addition of NaHMDS decreased side-product formation.

TLC: 0.45 (hexanes/EtOAc, 40% EtOAc, UV)¹H NMR: (500 MHz, CDCl₃) δ 7.41 (s, 2H), 5.04 (d, J=13.4 Hz, 1H), 4.43 (d, J=13.4 Hz, 1H), 1.49 (s, 9H), 1.18 (s, 9H) ppm. ¹³C NMR: (126 MHz, CDCl₃) δ 188.02, 150.86, 143.10, 125.10, 64.58, 49.53, 41.94, 27.65, 23.83 ppm. Specific rotation=−1.5 (c 1.00, CHCl₃) Melting point: 139° C. HRMS: Calc'd for C₁₅H₂₂C₁₂N₂O₂SNa [M+Na⁺] 387.0671; found: 387.0684.

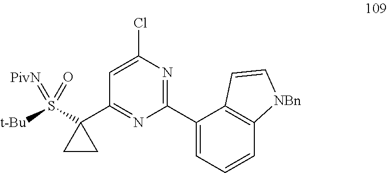

GP-4 was used with previously prepared

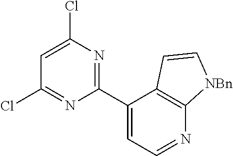 [SMILES: Clc1cc(Cl)nc(-c2ccnc3c2ccn3Cc2ccccc2)n1]

(−78° C. for 7 hours, removed from dry ice bath for 5 minutes then quenched). Purified by silica gel column chromatography using hexanes/EtOAc (0% to 30% EtOAc gradient) to give 109 (4.28 g, 7.59 mmol, 79% yield) as a yellow oil. Note: 1.5 eq. of NaHMDS was used.

TLC: R_{f}=0.19 (20% EtOAc in hexanes, UV)¹H NMR: (500 MHz, CDCl₃) δ 8.51 (d, J=5.1 Hz, 1H), 8.17 (d, J=5.0 Hz, 1H), 7.71 (s, 1H), 7.44 (d, J=3.5 Hz, 1H), 7.38 (d, J=3.5 Hz, 1H), 7.34-7.26 (m, 3H), 7.27-7.21 (m, 4H), 5.58 (d, J=3.8 Hz, 2H), 2.58 (ddd, J=10.5, 8.1, 5.9 Hz, 1H), 2.20-2.12 (m, 1H), 1.99 (ddd, J=10.5, 7.9, 5.5 Hz, 1H), 1.47-1.40 (m, 10H), 1.27 (s, 9H) ppm. ¹³C NMR: (126 MHz, CDCl₃) δ 188.03, 165.87, 164.87, 162.03, 149.36, 142.79, 137.45, 134.68, 130.19, 128.77, 127.76, 127.51, 122.69, 118.79, 115.68, 102.12, 66.73, 48.20, 42.32, 42.28, 27.81, 24.66, 16.64, 14.62 ppm. Specific rotation=−116.6 (c 1.00, CHCl₃) HRMS: Calc'd for C₃₀H₃₄ClN₅O₂SNa [M+Na⁺]586.2014; found: 586.2016.

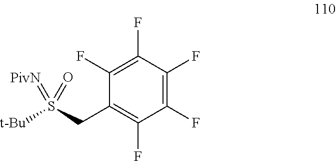

GP-4 was used with commercially available hexafluorobenzene (−78° C. for 1 hour, warmed to room temperature then stirred for 15 hours). Purified by silica gel column chromatography using hexanes/EtOAc (0% to 20% EtOAc gradient) to give 110 (86.0 mg, 0.223 mmol, 89% yield) as a colorless solid.

TLC: R_{f}=0.53 (hexanes/EtOAc, 40% EtOAc, UV). ¹H NMR: (500 MHz, CDCl₃) δ 5.02 (d, J=13.6 Hz, 1H), 4.51 (d, J=13.6 Hz, 1H), 1.55 (s, 9H), 1.14 (s, 9H) ppm. ¹³C NMR: (126 MHz, CDCl₃) δ 187.30, 147.85-144.78 (m), 143.59-140.45 (m), 139.33-136.19 (m), 103.72 (td, J=17.4, 4.1 Hz), 64.73, 41.88, 40.96, 27.63, 23.71 ppm. ¹⁹F NMR: (471 MHz, CDCl₃) δ −137.77-−137.95 (m), −151.46 (t, J=20.9 Hz), −161.28 (td, J=20.4, 6.1 Hz) ppm. Specific rotation=+2.33 (c 1.00, CHCl₃). HRMS: Calc'd for C₁₆H₁₅N₂O₂S₂ [M+H⁺] 386.1208; found: 386.1211.

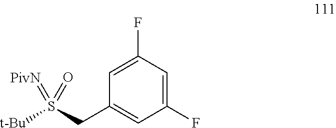

GP-5 was used with commercially available 1,3,5-trifluorobenzene (60° C. for 15 hours). Purified by silica gel column chromatography using hexanes/EtOAc (0% to 40% EtOAc gradient) to give 111 (64.0 mg, 0.193 mmol, 77% yield) as a white solid.

TLC: R_{f}=0.24 (hexanes/EtOAc, 40% EtOAc, UV). ¹H NMR: (500 MHz, CDCl₃) δ 7.04 (dd, J=7.8, 2.2 Hz, 2H), 6.81 (tt, J=8.8, 2.3 Hz, 1H), 5.05 (d, J=13.7 Hz, 1H), 4.57 (d, J=13.7 Hz, 1H), 1.45 (s, 9H), 1.18 (s, 9H) ppm. ¹³C NMR: (126 MHz, CDCl₃) δ 188.02, 163.83 (d, J=12.8 Hz), 161.84 (d, J=12.7 Hz), 131.97 (t, J=10.0 Hz), 114.32 (d, J=13.6 Hz), 114.32 (d, J=26.4 Hz), 104.57 (t, J=25.0 Hz), 64.03, 51.28 (t, J=2.1 Hz), 41.86, 27.71, 23.89 ppm. ¹⁹F NMR: (471 MHz, CDCl₃) δ −108.92 ppm. Specific rotation=+49.6 (c 0.66, CHCl₃). HRMS: Calc'd for C₁₆H₂₄NO₂S [M+H⁺] 332.1490; found: 332.1490.

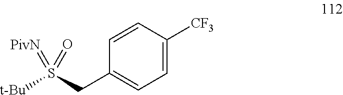

GP-5 was used with commercially available 1-fluoro-4-(trifluoromethyl)benzene (60° C. for 20 hours). Purified by silica gel column chromatography using hexanes/EtOAc (0% to 40% EtOAc gradient) to give 112 (46.0 mg, 0.127 mmol, 51% yield) as a white solid.

TLC: R_{f}=0.22 (hexanes/EtOAc, 20% EtOAc, UV). ¹H NMR: (500 MHz, CDCl₃) δ 7.62 (s, 4H), 5.15 (d, J=13.7 Hz, 1H), 4.63 (d, J=13.7 Hz, 1H), 1.45 (s, 9H), 1.18 (s, 9H) ppm. ¹³C NMR: (126 MHz, CDCl₃) δ 188.00, 132.68 (q, J=1.2 Hz), 131.70, 131.00 (q, J=32.6 Hz), 125.54 (q, J=3.7 Hz), 123.87 (q, J=271.9 Hz), 63.96, 51.47, 41.84, 27.75, 23.89 ppm. ¹⁹F NMR: (471 MHz, CDCl₃) δ −62.77 ppm. Specific rotation=+30.9 (c 0.54, CHCl₃) Melting point: 150-152° C. HRMS: Calc'd for C₁₇H₂₅F₃NO₂S [M+H⁺] 364.1553; found: 364.1553.

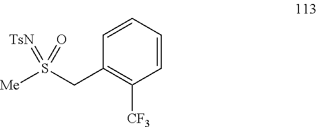

GP-5 was used with commercially available 1-fluoro-2-(trifluoromethyl)benzene (60° C. for 28 hours). Purified by silica gel column chromatography using hexanes/EtOAc (0% to 40% EtOAc gradient) to give 113 (52.0 mg, 0.133 mmol, 53% yield) as a colorless oil.

TLC: R_{f}=0.37 (hexanes/EtOAc, 40% EtOAc, UV). ¹H NMR: (500 MHz, CDCl₃) δ 7.94 (d, J=7.8 Hz, 1H), 7.89 (d, J=8.3 Hz, 2H), 7.77 (d, J=7.8 Hz, 1H), 7.67 (t, J=7.4 Hz, 1H), 7.57 (t, J=7.7 Hz, 1H), 7.32-7.27 (m, 2H), 5.04 (d, J=15.0 Hz, 1H), 4.98 (d, J=15.0 Hz, 1H), 2.97 (s, 3H), 2.42 (s, 3H). ¹³C NMR: (126 MHz, CDCl₃) δ 143.12, 140.52, 133.77, 132.77, 130.02, 129.71 (q, J=30.1 Hz), 129.38, 126.86 (q, J=5.5 Hz), 126.70, 125.11 (q, J=1.5 Hz), 123.83 (q, J=275.1 Hz), 58.21 (q, J=2.8 Hz), 40.50 (q, J=2.3 Hz), 21.57. ¹⁹F NMR: (471 MHz, CDCl₃) δ −57.52 ppm. HRMS: Calc'd for C₁₆H₁₇F₃NO₃S₂ [M+H⁺] 392.0596; found: 392.0596.

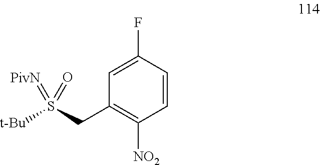

Modified GP-4: (R)-23 (0.066 g, 0.30 mmol, 1.2 eq.) in THF (2.0 mL) was cooled to −78° C. then NaHMDS (0.275 mL, 0.550 mmol, 2M in THF, 2.2 eq.) was added dropwise and stirred at −78° C. for 35 minutes. A solution of commercially available 2,4-difluoro-1-nitrobenzene (0.039 g, 0.250 mmol, 1 eq.) in THF (0.5 mL) was added slowly over 10 minutes. The resulting reaction mixture was stirred at −78° C. for 5 hours before warming to room temperature where it stirred for 10 hours. The reaction was quenched with saturated NH₄Cl (8 mL) and water (5 mL), extracted with EtOAc (4×15 mL), combined organic layers dried over Na₂SO₄, filtered and concentrated. Purified by silica gel column chromatography using hexanes/EtOAc (0% to 20% EtOAc gradient) to give 114 (61.0 mg, 0.170 mmol, 68% yield) as a white solid.

TLC: R_{f}=0.64 (hexanes/EtOAc, 40% EtOAc, UV). ¹H NMR: (500 MHz, CDCl₃) δ 8.12 (dd, J=9.1, 5.1 Hz, 1H), 7.53 (dd, J=8.9, 2.8 Hz, 1H), 7.23-7.17 (m, 1H), 5.45 (d, J=13.2 Hz, 1H), 5.33 (d, J=13.2 Hz, 1H), 1.50 (s, 9H), 1.13 (s, 9H) ppm. ¹³C NMR: (126 MHz, CDCl₃) δ 187.32, 164.25 (d, J=257.9 Hz), 145.98 (d, J=2.7 Hz), 128.23 (d, J=9.8 Hz), 126.47 (d, J=9.9 Hz), 121.80 (d, J=24.9 Hz), 116.89 (d, J=23.0 Hz), 64.66, 46.42, 41.89, 27.64, 23.67 ppm. ¹⁹F NMR: (471 MHz, CDCl₃) δ −102.54 ppm. Specific rotation=−233.1 (c 0.67, CHCl₃). HRMS: Calc'd for C₁₆H₂₃FN₂O₄SNa [M+Na⁺] 381.1255; found: 381.1265.

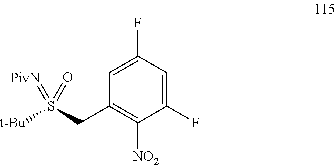

Modified GP-4 used from directly above. Commercially available 2,4,6-trifluoro-1-nitrobenzene was used. Purified by silica gel column chromatography using hexanes/EtOAc (0% to 20% EtOAc gradient) to give 115 (51.0 mg, 0.135 mmol, 54% yield) as a white solid.

TLC: R_{f}=0.63 (hexanes/EtOAc, 40% EtOAc, UV). ¹H NMR: (500 MHz, CDCl₃) δ 7.44 (dt, J=8.8, 2.2 Hz, 1H), 7.04 (ddd, J=10.2, 7.7, 2.7 Hz, 1H), 5.27 (d, J=13.7 Hz, 1H), 4.88 (d, J=13.7 Hz, 1H), 1.47 (s, 9H), 1.16 (s, 9H) ppm. ¹³C NMR: (126 MHz, CDCl₃) δ 187.49, 162.88 (dd, J=257.9, 12.2 Hz), 155.70 (dd, J=262.7, 13.4 Hz), 137.00 (dd, J=11.9, 4.1 Hz), 126.47 (d, J=11.0 Hz), 116.68 (dd, J=24.8, 3.7 Hz), 106.49 (dd, J=26.6, 23.8 Hz), 64.65, 45.47, 41.95, 27.62, 23.56 ppm. ¹⁹F NMR: (471 MHz, CDCl₃) δ −100.56 (d, J=10.7 Hz), −114.71 (d, J=10.8 Hz) ppm. Specific rotation=−153.8 (c 0.73, CHCl₃) HRMS: Calc'd for C₁₆H₂₂F₂N₂O₄SNa [M+Na⁺] 399.1161; found: 399.1168.

### REFERENCES

Each of the below references is incorporated by reference herein in its entirety for at least the material for which it is cited.
(1) Ilardi, E. A.; Vitaku, E.; Njardarson, J. T., Data-Mining for Sulfur and Fluorine: An Evaluation of Pharmaceuticals To Reveal Opportunities for Drug Design and Discovery. J. Med. Chem. 2014, 57 (7), 2832-2842.
(2) Zhao, C.; Rakesh, K. P.; Ravidar, L.; Fang, W.-Y.; Qin, H.-L., Pharmaceutical and medicinal significance of sulfur (S(VI))-Containing motifs for drug discovery: A critical review. Eur. J. Med. Chem. 2019, 162, 679-734.
(3) Tang, K.-X.; Wang, C.-M.; Gao, T.-H.; Chen, L.; Fan, L.; Sun, L.-P., Transition Metal-Catalyzed C—H Bond Functionalizations by Use of Sulfur-Containing Directing Groups. Adv. Synth. Catal. 2019, 361 (1), 26-38.
(4) Wang, J.; Lakraychi, A. E.; Liu, X.; Sieuw, L.; Morari, C.; Poizot, P.; Vlad, A., Conjugated sulfonamides as a class of organic lithium-ion positive electrodes. Nat. Mater. 2021, 20 (5), 665-673.
(5) Scott, K. A.; Njardarson, J. T., Analysis of US FDA-Approved Drugs Containing Sulfur Atoms. Top Curr Chem. 2018, 376, 5.
(6) Mäder, P.; Kattner, L., Sulfoximines as Rising Stars in Modern Drug Discovery? Current Status and Perspective on an Emerging Functional Group in Medicinal Chemistry. J. Med. Chem. 2020, 63 (23), 14243-14275.
(7) Lücking, U., Sulfoximines: A Neglected Opportunity in Medicinal Chemistry. Angew. Chem. Int. Ed. 2013, 52 (36), 9399-9408.
(8) Sirvent, J. A.; Lücking, U., Novel Pieces for the Emerging Picture of Sulfoximines in Drug Discovery: Synthesis and Evaluation of Sulfoximine Analogues of Marketed Drugs and Advanced Clinical Candidates. ChemMedChem 2017, 12 (7), 487-501.
(9) Lücking, U., Neglected sulfur(VI) pharmacophores in drug discovery:
exploration of novel chemical space by the interplay of drug design and method development. Org. Chem. Front. 2019, 6 (8), 1319-1324.
(10) Devendar, P.; Yang, G. F., Sulfur-Containing Agrochemicals. Top Curr Chem. 2017, 375 (6), 82.
(11) Zasukha, S. V.; Timoshenko, V. M.; Tolmachev, A. A.; Pivnytska, V. O.; Gavrylenko, O.; Zhersh, S.; Shermolovich, Y.; Grygorenko, O. O., Sulfonimidamides and Imidosulfuric Diamides: Compounds from an Underexplored Part of Biologically Relevant Chemical Space. Chem.—Eur. J. 2019, 25 (28), 6928-6940.
(12) Chinthakindi, P. K.; Naicker, T.; Thota, N.; Govender, T.; Kruger, H. G.; Arvidsson, P. I., Sulfonimidamides in Medicinal and Agricultural Chemistry. Angew. Chem. Int. Ed. 2017, 56 (15), 4100-4109.
(13) Foote, K. M.; Nissink, J. W. M.; McGuire, T.; Turner, P.; Guichard, S.; Yates, J. W. T.; Lau, A.; Blades, K.; Heathcote, D.; Odedra, R.; Wilkinson, G.; Wilson, Z.; Wood, C. M.; Jewsbury, P. J., Discovery and Characterization of AZD6738, a Potent Inhibitor of Ataxia Telangiectasia Mutated and Rad3 Related (ATR) Kinase with Application as an Anticancer Agent. J. Med. Chem. 2018, 61 (22), 9889-9907.
(14) Luecking, U.; Hog, D.; Geisler, J.; Scholz, A.; Petersen, K.; Lienau, P.; Stegmann, C.; Andres, D.; Siemeister, G.; Werbeck, N. Preparation of modified macrocyclic compounds for the treatment of diseases. PCT Int. Patent Appl. WO 2017/060167 A1, Apr. 13, 2017.
(15) Luecking, U.; Geisler, J.; Hog, D.; Scholz, A.; Petersen, K.; Lienau, P.; Stegmann, C.; Andres, D.; Zheng, K.; Gao, P.; Chen, G.; Xi, J.; Herbert, S.; Siemeister, G.; Werbeck, N. Preparation of macrocyclic sulfondiimine compounds as CDK9 inhibitor for treatment of hyperproliferative disorder, viral infection, and cardiovascular diseases. PCT Int. Patent Appl. WO 2017/055196 A1, Jun. 4, 2017.
(16) Zhu, Y.; Loso, M. R.; Watson, G. B.; Sparks, T. C.; Rogers, R. B.; Huang, J. X.; Gerwick, B. C.; Babcock, J. M.; Kelley, D.; Hegde, V. B.; Nugent, B. M.; Renga, J. M.; Denholm, I.; Gorman, K.; DeBoer, G. J.; Hasler, J.; Meade, T.; Thomas, J. D., Discovery and Characterization of Sulfoxaflor, a Novel Insecticide Targeting Sap-Feeding Pests. J. Agric. Food Chem. 2011, 59 (7), 2950-2957.
(17) Johnson, T.; Vairagoundar, R.; Ewin, R. A. Preparation of novel phenicol derivatives useful as antibacterial agents. PCT Int. Patent Appl. WO 2014/172443 A1, Oct. 23, 2014.
(18) Lücking, U.; Kosemund, D.; Böhnke, N.; Lienau, P.; Siemeister, G.; Denner, K.; Bohlmann, R.; Briem, H.; Terebesi, I.; Bömer, U.; Schafer, M.; Ince, S.; Mumberg, D.; Scholz, A.; Izumi, R.; Hwang, S.; von Nussbaum, F., Changing for the Better: Discovery of the Highly Potent and Selective CDK9 Inhibitor VIP152 Suitable for Once Weekly Intravenous Dosing for the Treatment of Cancer. J. Med. Chem. 2021, 64 (15), 11651-11674.
(19) Frings, M.; Bolm, C.; Blum, A.; Gnamm, C., Sulfoximines from a Medicinal Chemist's Perspective: Physicochemical and in vitro Parameters Relevant for Drug Discovery. Eur. J. Med. Chem. 2017, 126, 225-245.
(20) Lücking, U.; Jautelat, R.; Kruger, M.; Brumby, T.; Lienau, P.; Schafer, M.; Briem, H.; Schulze, J.; Hillisch, A.; Reichel, A.; Wengner, A. A. M.; Siemeister, G., The Lab Oddity Prevails: Discovery of Pan-CDK Inhibitor (R)—S-Cyclopropyl-S-(4-{[4-{[(1R,2R)-2-hydroxy-1-methylpropyl]oxy}-5-(trifluoromethyl)pyrimidin-2-yl]amino}phenyl)sulfoximide (BAY 1000394) for the Treatment of Cancer. ChemMedChem 2013, 8 (7), 1067-1085.
(21) Izzo, F.; Schafer, M.; Lienau, P.; Ganzer, U.; Stockman, R.; Lücking, U., Exploration of Novel Chemical Space: Synthesis and in vitro Evaluation of N-Functionalized Tertiary Sulfonimidamides. Chem.—Eur. J. 2018, 24 (37), 9295-9304.
(22) Clinical Trials Using ATR Kinase Inhibitor AZD6738. https://www.cancer.gov/about-cancer/treatment/clinical-trials/intervention/atr-kinase-inhibitor-azd6738 (accessed May 26, 2021).
(23) Luecking, U.; Hog, D.; Christ, C.; Sack, U.; Siegel, F.; Lienau, P.; Werbeck, N. Preparation of novel PTEFB inhibiting macrocyclic compounds. PCT Int. Patent Appl. WO 2018/177899 A1, Mar. 22, 2018.
(24) Walker, D. P.; Zawistoski, M. P.; McGlynn, M. A.; Li, J. C.; Kung, D. W.; Bonnette, P. C.; Baumann, A.; Buckbinder, L.; Houser, J. J. A.; Boer, J.; Mistry, A.; Han, S.; Xing, L.; Guzman-Perez, A., Sulfoximine-substituted trifluoromethylpyrimidine analogs as inhibitors of proline-rich tyrosine kinase 2 (PYK2) show reduced hERG activity. Bioorg. Med. Chem. Lett. 2009, 19 (12), 3253-8.
(25) Mc Bride, C.; Trzoss, L. L.; Boloor, A.; Sokolova, N.; Pastor, R. M.; Staben, S. T.; Stivala, C.; Volgraf, M.; Bronner, S. M. Preparation of sulfonimidamide compounds as inhibitors of interleukin-1 activity. PCT Int. Patent Appl. WO 2020/018975 A1, Jan. 23, 2020.
(26) Chowdhury, S.; Dehnhardt, C. M.; Focken, T.; Grimwood, M. E.; Hemeon, I. W.; McKerrall, S.; Sutherlin, D. Preparation of substituted benzamides as sodium channel inhibitors and therapeutic uses thereof. PCT Int. Patent Appl. WO 2017/172802 A1, May 10, 2017.
(27) Bartels, B.; Dolente, C.; Guba, W.; Haap, W.; Kuglstatter, A.; Obst Sander, U.; Peters, J.-U.; Rogers-Evans, M.; Woltering, T.; Schnider, C.; Wermuth, R. Preparation of pyridyl-triazabicycles having BACE1 inhibitory activity. PCT Int. Patent Appl. WO 2016/012422 A1, Jan. 28, 2016.
(28) Katz, J.; Roush, W.; Seidel, H. M.; Shen, D.-M.; Venkatraman, S. Sulfonimidamide compounds and compositions for treating conditions associated with NLRP activity. PCT Int. Patent Appl. WO 2020/154499 A1, Jul. 30, 2020.
(29) Ouvry, G.; Bihl, F.; Bouix-Peter, C.; Christin, O.; Defoin-Platel, C.; Deret, S.; Feret, C.; Froude, D.; Hacini-Rachinel, F.; Harris, C. S.; Hervouet, C.; Lafitte, G.; Luzy, A.-P.; Musicki, B.; Orfila, D.; Parnet, V.; Pascau, C.; Pascau, J.; Pierre, R.; Raffin, C.; Rossio, P.; Spiesse, D.; Taquet, N.; Thoreau, E.; Vatinel, R.; Vial, E.; Hennequin, L. F., Sulfoximines as potent RORγ inverse agonists. Bioorg. Med. Chem. Lett. 2018, 28 (8), 1269-1273.
(30) Loso, M. R.; Benko, Z.; Buysse, A.; Johnson, T. C.; Nugent, B. M.; Rogers, R. B.; Sparks, T. C.; Wang, N. X.; Watson, G. B.; Zhu, Y., SAR studies directed toward the pyridine moiety of the sap-feeding insecticide sulfoxaflor (Isoclast active). Bioorg. Med. Chem. 2016, 24 (3), 378-382.
(31) SciFinder; Chemical Abstracts Service: Columbus, OH; Patent search for sulfoximine-containing compounds: structure search using general sulfoximine; https://scifinder-n.cas.org/search/reference/60b0d9dde91ebe7b813ae2f8/1 (accessed May 28).
(32) SciFinder; Chemical Abstracts Service: Columbus, OH; Patent search for sulfonimidamide-containing compounds: structure search using general sulfonimidamide; https://scifinder-n.cas.org/search/reference/60b0da8be91ebe7b813ae3af/1 (accessed May 28).
(33) SciFinder; Chemical Abstracts Service: Columbus, OH; Patent search for sulfondiimine-containing compounds: structure search using general sulfondiimine; https://scifinder-n.cas.org/search/reference/60b0dae9e91ebe7b813ae41a/1 (accessed May 28).
(34) Okamura, H.; Bolm, C., Rhodium-Catalyzed Imination of Sulfoxides and Sulfides: Efficient Preparation of N-Unsubstituted Sulfoximines and Sulfilimines. Org. Lett. 2004, 6 (8), 1305-1307.
(35) Correa, A.; Bolm, C., Ligand-free copper-catalyzed N-arylation of nitrogen nucleophiles. Adv. Synth. Catal. 2007, 349 (17+18), 2673-2676.
(36) Cheng, Y.; Bolm, C., Regioselective Syntheses of 1,2-Benzothiazines by Rhodium-Catalyzed Annulation Reactions. Angew. Chem. Int. Ed. 2015, 54 (42), 12349-12352.
(37) Zenzola, M.; Doran, R.; Luisi, R.; Bull, J. A., Synthesis of Sulfoximine Carbamates by Rhodium-Catalyzed Nitrene Transfer of Carbamates to Sulfoxides. J. Org. Chem. 2015, 80 (12), 6391-6399.
(38) Zenzola, M.; Doran, R.; Degennaro, L.; Luisi, R.; Bull, J. A., Transfer of Electrophilic NH Using Convenient Sources of Ammonia: Direct Synthesis of NH Sulfoximines from Sulfoxides. Angew. Chem., Int. Ed. 2016, 55 (25), 7203-7207.
(39) Tota, A.; Zenzola, M.; Chawner, S. J.; John-Campbell, S. S.; Carlucci, C.; Romanazzi, G.; Degennaro, L.; Bull, J. A.; Luisi, R., Synthesis of NH-sulfoximines from sulfides by chemoselective one-pot N- and O-transfers. Chem. Commun. 2017, 53 (2), 348-351.
(40) Johnson, C. R., Utilization of sulfoximines and derivatives as reagents for organic synthesis. Acc. Chem. Res. 1973, 6 (10), 341-347
(41) Sirvent, J. A.; Bierer, D.; Webster, R.; Lücking, U., Palladium-Catalyzed Direct α-Arylation of p-Methoxybenzyl-Protected S,S-Dimethylsulfoximine. Synthesis 2017, 49 (05), 1024-1036.
(42) Greed, S.; Briggs, E. L.; Idiris, F. I. M.; White, A. J. P.; Luecking, U.; Bull, J. A., Synthesis of Highly Enantioenriched Sulfonimidoyl Fluorides and Sulfonimidamides by Stereospecific Sulfur-Fluorine Exchange (SuFEx) Reaction. Chem.—Eur. J. 2020, 26 (55), 12533-12538.
(43) Izzo, F.; Schaefer, M.; Stockman, R.; Luecking, U., A New, Practical One-Pot Synthesis of Unprotected Sulfonimidamides by Transfer of Electrophilic NH to Sulfinamides. Chem.—Eur. J. 2017, 23 (60), 15189-15193.
(44) Aota, Y.; Kano, T.; Maruoka, K., Asymmetric Synthesis of Chiral Sulfoximines through the S-Alkylation of Sulfinamides. Angew. Chem. Int. Ed. 2019, 58 (49), 17661-17665.
(45) Aota, Y.; Kano, T.; Maruoka, K., Asymmetric Synthesis of Chiral Sulfoximines via the S-Arylation of Sulfinamides. J. Am. Chem. Soc. 2019, 141 (49), 19263-19268.
(46) Gao, B.; Li, S.; Wu, P.; Moses, J. E.; Sharpless, K. B., SuFEx Chemistry of Thionyl Tetrafluoride (SOF4) with Organolithium Nucleophiles: Synthesis of Sulfonimidoyl Fluorides, Sulfoximines, Sulfonimidamides, and Sulfonimidates. Angew. Chem., Int. Ed. 2018, 57 (7), 1939-1943.
(47) Zheng, Q.; Woehl, J. L.; Kitamura, S.; Santos-Martins, D.; Smedley, C. J.; Li, G.; Forli, S.; Moses, J. E.; Wolan, D. W.; Sharpless, K. B., SuFEx-enabled, agnostic discovery of covalent inhibitors of human neutrophil elastase. Proc. Natl. Acad. Sci. U.S.A. 2019, 116 (38), 18808.
(48) Kitamura, S.; Zheng, Q.; Woehl, J. L.; Solania, A.; Chen, E.; Dillon, N.; Hull, M. V.; Kotaniguchi, M.; Cappiello, J. R.; Kitamura, S.; Nizet, V.; Sharpless, K. B.; Wolan, D. W., Sulfur(VI) Fluoride Exchange (SuFEx)-Enabled High-Throughput Medicinal Chemistry. J. Am. Chem. Soc. 2020, 142 (25), 10899-10904.
(49) Davies, T. Q.; Hall, A.; Willis, M. C., One-Pot, Three-Component Sulfonimidamide Synthesis Exploiting the Sulfinylamine Reagent N-Sulfinyltritylamine, TrNSO. Angew. Chem. Int. Ed. 2017, 56 (47), 14937-14941.
(50) Zhang, Z.-X.; Davies, T. Q.; Willis, M. C., Modular Sulfondiimine Synthesis Using a Stable Sulfinylamine Reagent. J. Am. Chem. Soc. 2019, 141 (33), 13022-13027.
(51) Davies, T. Q.; Tilby, M. J.; Ren, J.; Parker, N. A.; Skolc, D.; Hall, A.; Duarte, F.; Willis, M. C., Harnessing Sulfinyl Nitrenes: A Unified One-Pot Synthesis of Sulfoximines and Sulfonimidamides. J. Am. Chem. Soc. 2020, 142 (36), 15445-15453.
(52) Pyne, S. G.; Dong, Z.; Skelton, B. W.; White, A. H., Cyclopropanation reactions of enones with lithiated sulfoximines: application to the asymmetric synthesis of chiral cyclopropanes. J. Org. Chem. 1997, 62 (8), 2337-2343.
(53) Zhang, W.; Wang, F.; Hu, J., N-Tosyl-S-difluoromethyl-S-phenylsulfoximine: A New Difluoromethylation Reagent for S-, N-, and C-Nucleophiles. Org. Lett. 2009, 11 (10), 2109-2112.
(54) Shen, X.; Liu, Q.; Zhang, W.; Hu, J., Stereoselective Synthesis of (Sulfonimidoyl)cyclopropanes with (R)-PhSO(NTs)CH2Cl and α,β-Unsaturated Weinreb Amides: Tuning the of Selectivity between C—Cl and C—S Bond Cleavage. Eur. J. Org. Chem. 2016, 2016 (5), 906-909.
(55) Cho, G. Y.; Bolm, C., Palladium-catalyzed α-arylation of sulfoximines. Org. Lett. 2005, 7 (7), 1351-1354.
(56) Leftheris, K.; Zhuang, L.; Tice, C. M.; Singh, S. B.; Ye, Y.; Xu, Z.; Himmelsbach, F.; EcKhardt, M. Substituted 5-,6- and 7-membered heterocycles as 110-HSD1 inhibitors and their preparation, medicaments containing such compounds, and their use. PCT Int. Patent Appl. WO 2011/159760 A1, Dec. 22, 2011
(57) Liu, Y.; Xia, Q.; Fang, L., Design and synthesis of alkyl substituted pyridino[2,3-D]pyrimidine compounds as PI3Kα/mTOR dual inhibitors with improved pharmacokinetic properties and potent in vivo antitumor activity. Bioorg. Med. Chem. 2018, 26 (14), 3992-4000.
(58) Blades, K.; Demeritt, J.; Fillery, S.; Foote, K. M.; Greenwood, R.; Gregson, C.; Hassall, L. A.; McGuire, T. M.; Pike, K. G.; Williams, E., Expedient synthesis of biologically important sulfonylmethyl pyrimidines. Tetrahedron Lett. 2014, 55 (29), 3851-3855.
(59) Haake, M., Ein neues verfahren zur darstellung von S,S-dialkylschwefeldiimiden. Tetrahedron Lett. 1970, 11 (51), 4449-4450.
(60) Park, S. J.; Baars, H.; Mersmann, S.; Buschmann, H.; Baron, J. M.; Amann, P. M.; Czaja, K.; Hollert, H.; Bluhm, K.; Redelstein, R.; Bolm, C., N-cyano sulfoximines: COX inhibition, anticancer activity, cellular toxicity, and mutagenicity. ChemMedChem 2013, 8 (2), 217-20.
(61) Murray, J. M.; Sweeney, Z. K.; Chan, B. K.; Balazs, M.; Bradley, E.; Castanedo, G.; Chabot, C.; Chantry, D.; Flagella, M.; Goldstein, D. M.; Kondru, R.; Lesnick, J.; Li, J.; Lucas, M. C.; Nonomiya, J.; Pang, J.; Price, S.; Salphati, L.; Safina, B.; Savy, P. P. A.; Seward, E. M.; Ultsch, M.; Sutherlin, D. P., Potent and Highly Selective Benzimidazole Inhibitors of PI3-Kinase Delta. J. Med. Chem. 2012, 55 (17), 7686-7695.
(62) Kang, D.; Fang, Z.; Huang, B.; Lu, X.; Zhang, H.; Xu, H.; Huo, Z.; Zhou, Z.; Yu, Z.; Meng, Q.; Wu, G.; Ding, X.; Tian, Y.; Daelemans, D.; De Clercq, E.; Pannecouque, C.; Zhan, P.; Liu, X., Structure-Based Optimization of Thiophene[3,2-d]pyrimidine Derivatives as Potent HIV-1 Non-nucleoside Reverse Transcriptase Inhibitors with Improved Potency against Resistance-Associated Variants. J. Med. Chem. 2017, 60 (10), 4424-4443.
(63) Goundry, W. R. F.; Dai, K.; Gonzalez, M.; Legg, D.; O'Kearney-McMullan, A.; Morrison, J.; Stark, A.; Siedlecki, P.; Tomlin, P.; Yang, J., Development and Scale-up of a Route to ATR Inhibitor AZD6738. Org. Process Res. Dev. 2019, 23 (7), 1333-1342.
(64) Graham, M. A.; Askey, H.; Campbell, A. D.; Chan, L.; Cooper, K. G.; Cui, Z.; Dalgleish, A.; Dave, D.; Ensor, G.; Galan Espinosa, M. R.; Hamilton, P.; Heffernan, C.; Jackson, L. V.; Jing, D.; Jones, M. F.; Liu, P.; Mulholland, K. R.; Pervez, M.; Popadynec, M.; Randles, E.; Tomasi, S.; Wang, S., Development and Scale-Up of an Improved Manufacturing Route to the ATR Inhibitor Ceralasertib. Org. Process Res. Dev. 2021, 25 (1), 43-56.
(65) Graham, M. A.; Noonan, G.; Cherryman, J. H.; Douglas, J. J.; Gonzalez, M.; Jackson, L. V.; Leslie, K.; Liu, Z.-q.; McKinney, D.; Munday, R. H.; Parsons, C. D.; Whittaker, D. T. E.; Zhang, E.-x.; Zhang, J.-w., Development and Proof of Concept for a Large-Scale Photoredox Additive-Free Minisci Reaction. Org. Process Res. Dev. 2021, 25 (1), 57-67.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention with departing from the scope thereof, it is to be understood that all matter herein set forth or shown in the accompanying drawings is to be interpreted as illustrative and not in a limiting sense.

1. A process for the preparation of a compound of Formula I 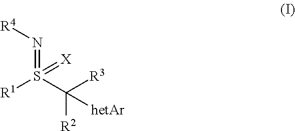
comprising reacting a compound of Formula II 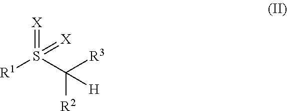
with a compound of Formula III 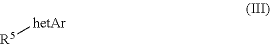
in the presence of a base;
wherein:
X is independently selected at each occurrence from O or NR⁴;
R¹ is selected from C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), (5- to 10-membered monocyclic or bicyclic heteroaryl)(C₀-C₃alkyl), or NR⁶R⁷, each of which may be optionally substituted as allowed by valence with one or more Y or Z groups;
R² and R³ are independently selected from hydrogen, C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), or (5- to 10-membered monocyclic or bicyclic heteroaryl)(C₀-C₃alkyl), each of which may be optionally substituted as allowed by valence with one or more Y or Z groups;
R⁴ is selected at each occurrence from hydrogen, C₁-C₆alkyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), (5- to 10-membered monocyclic or bicyclic heteroaryl)(C₀-C₃alkyl), C(═O)R⁸, or Si(R⁹)(R¹⁰)(R¹¹), each of which may be optionally substituted as allowed by valence with one or more Y or Z groups;
or R⁴ is a nitrogen protecting group;
or R¹ and R² may be brought together with the atoms to which they are attached to form a cycloalkyl or heterocycle ring, each of which may be optionally substituted as allowed by valence with one or more Y or Z groups;
or R² and R³ may be brought together with the carbon to which they are attached to form a cycloalkyl or heterocycle ring, each of which may be optionally substituted as allowed by valence with one or more Y or Z groups;
or R¹ and R⁴ may be brought together with the atoms to which they are attached to form a monocyclic or bicyclic heterocycle or heteroaryl ring, each of which may be optionally substituted as allowed by valence with one or more Y or Z groups;
R⁵ is a leaving group, for example a halo, a sulfonate, or a sulfone group;
hetAr is a monocyclic or bicyclic 6- to 10-membered heteroaryl group having one or more ring heteroatoms selected from N, O, and S, wherein at least one of the ring heteroatoms is N, and wherein hetAr may be optionally substituted as allowed by valence with one or more Y groups;
R⁶ and R⁷ are each selected from hydrogen, C₁-C₆alkyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), and (5- to 10-membered monocyclic or bicyclic heteroaryl)(C₀-C₃alkyl), each of which may be optionally substituted with one or more Y or Z groups;
R⁸ is selected from hydrogen, C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), (5- to 10-membered monocyclic or bicyclic heteroaryl)(C₀-C₃alkyl), —OR^{a}, or —NR^{a}R^{b}, each of which may be optionally substituted as allowed by valence with one or more Y or Z groups;
R⁹, R¹⁰, and R¹¹ are independently selected from hydrogen, C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), and (5- to 10-membered monocyclic or bicyclic heteroaryl)(C₀-C₃alkyl), each of which may be optionally substituted as allowed by valence with one or more Y or Z groups;
Y is selected at each occurrence from halo, nitro, cyano, azido, oxo, C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), (5- to 10-membered monocyclic or bicyclic heteroaryl)(C₀-C₃alkyl), —OR^{a}, —SR^{a}, —NR^{a}R^{b}, —S(O)R^{c}, and —S(O)₂R^{c}, each of which may be optionally substituted with one or more Z groups as allowed by valence;
R^{a} and R^{b} are independently selected at each occurrence from hydrogen, C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), and (5- to 10-membered monocyclic or bicyclic heteroaryl)(C₀-C₃alkyl), each of which may be optionally substituted as allowed by valence with one or more Z groups;
R^{c} is independently selected at each occurrence from hydrogen, C₁-C₆alkyl, C₂-C₆alkenyl, C₂-C₆alkynyl, (C₃-C₇cycloalkyl)(C₀-C₃alkyl), (C₃-C₇heterocycle)(C₀-C₃alkyl), (6- to 10-membered monocyclic or bicyclic aryl)(C₀-C₃alkyl), (5- to 10-membered monocyclic or bicyclic heteroaryl)(C₀-C₃alkyl), —OR^{a}, —SR^{a}, and —NR^{a}R^{b}, each of which may be optionally substituted as allowed by valency with one or more Z groups; and
Z is independently selected at each occurrence from alkyl, haloalkyl, alkoxy, alkenyl, alkynyl, aryl, heteroaryl, aldehyde, amino, carboxylic ester, ether, halo, hydroxyl, ketone, cyano, silyl, sulfo-oxo, sulfonyl, sulfoxide, sulfonamide, thiol, or combinations thereof.

2. The process of claim 1, wherein the base comprises an alkali metal or alkaline earth metal salt of an amide base.

3. The process of claim 1, wherein the base comprises sodium bis(trimethylsilyl)amide, lithium bis(trimethylsilyl)amide, or potassium bis(trimethylsilyl)amide.

4. (canceled)

5. The process of claim 1, wherein the base comprises a metal hydride salt.

6. The process of claim 1, wherein the base comprises sodium hydride, potassium hydride, or cesium hydride.

7. (canceled)

8. The process of claim 1, wherein the reaction is performed further in the presence of a crown ether.

9. The process of claim 8, wherein the crown ether is selected from the group consisting of 15-crown-5, 18-crown-6, and 21-crown-7.

10. (canceled)

11. The process of claim 1, wherein the reaction is performed in the presence of a solvent.

12. The process of claim 11, wherein the solvent comprises an ethereal solvent.

13. The process of claim 12, wherein the ethereal solvent is selected from tetrahydrofuran or diethyl ether.

14. The process of claim 12, wherein the ethereal solvent is 1,4-dioxane.

15. The process of claim 1, wherein the base is sodium bis(trimethylsilyl)amide, and wherein the reaction is performed in the presence of tetrahydrofuran.

16. The process of claim 15, wherein the reaction is performed at a temperature ranging from about −78° C. to about 25° C.

17. The process of claim 1, wherein the base is sodium hydride, wherein the reaction is performed in the presence of 1,4-dioxane and 15-crown-5.

18. The process of claim 17, wherein the reaction is performed at a temperature ranging from about 25° C. to about 80° C.

19. The process of claim 1, wherein the compound of Formula II is selected from the group consisting of:

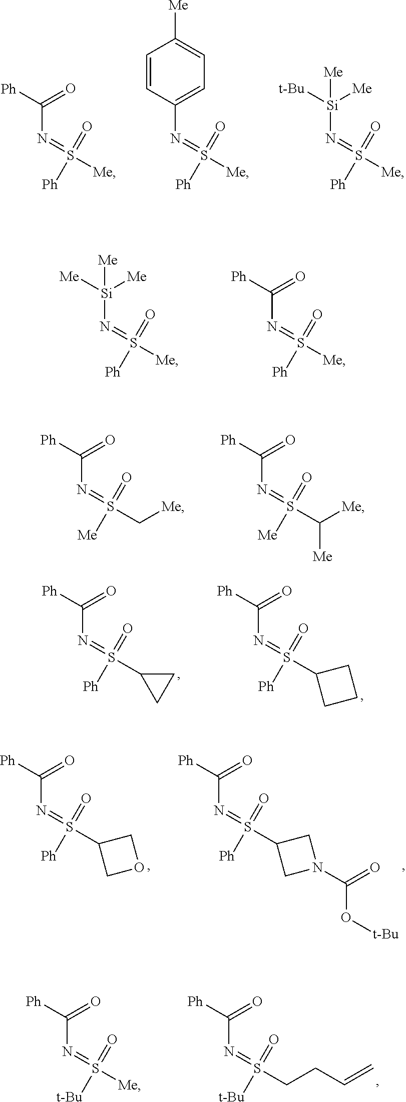

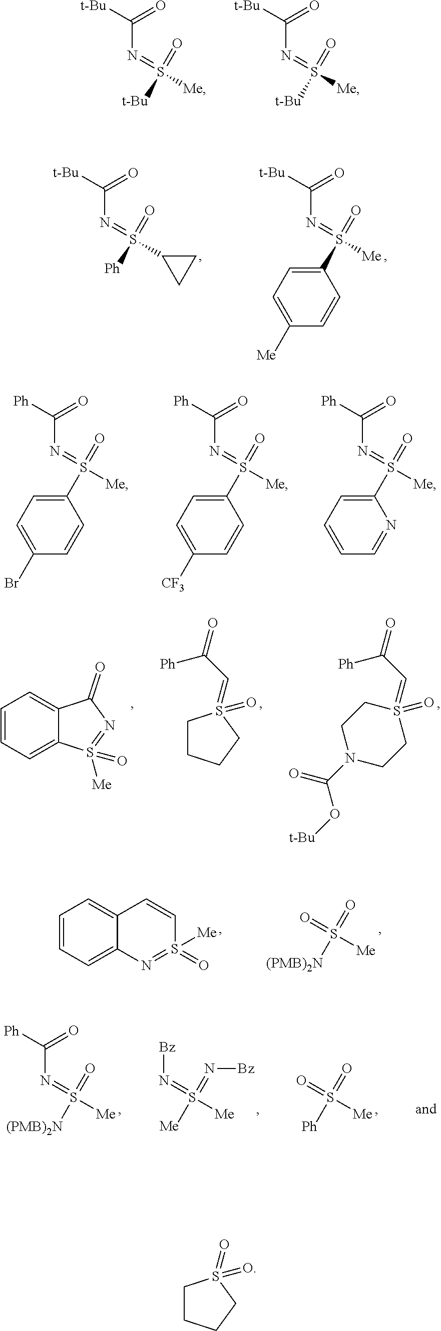

20. The process of claim 1, wherein the compound of Formula II is selected from the group consisting of:

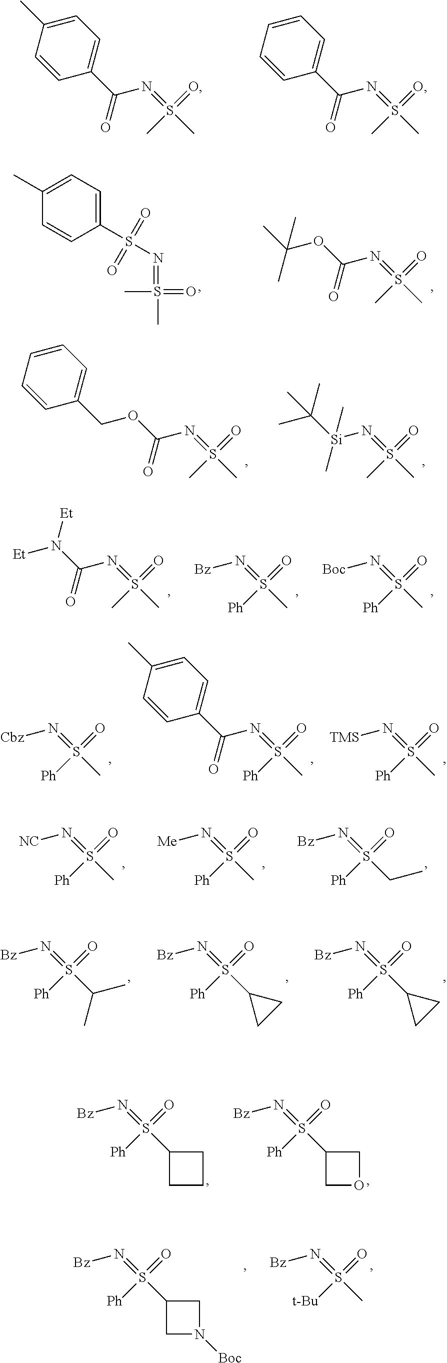

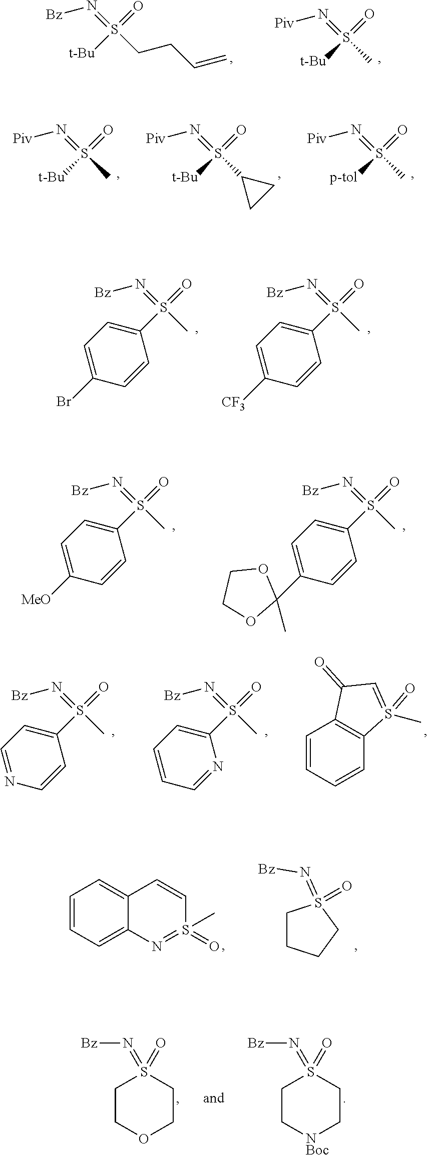

21. The process of claim 1, wherein the compound of Formula III is selected from the group consisting of:

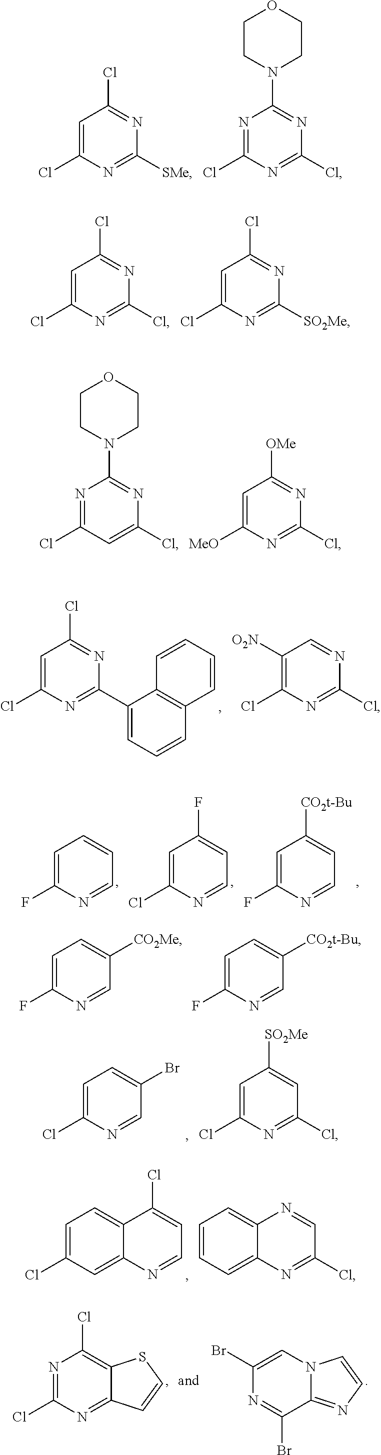

22. The process of claim 1, wherein the compound of Formula III is selected from the group consisting of:

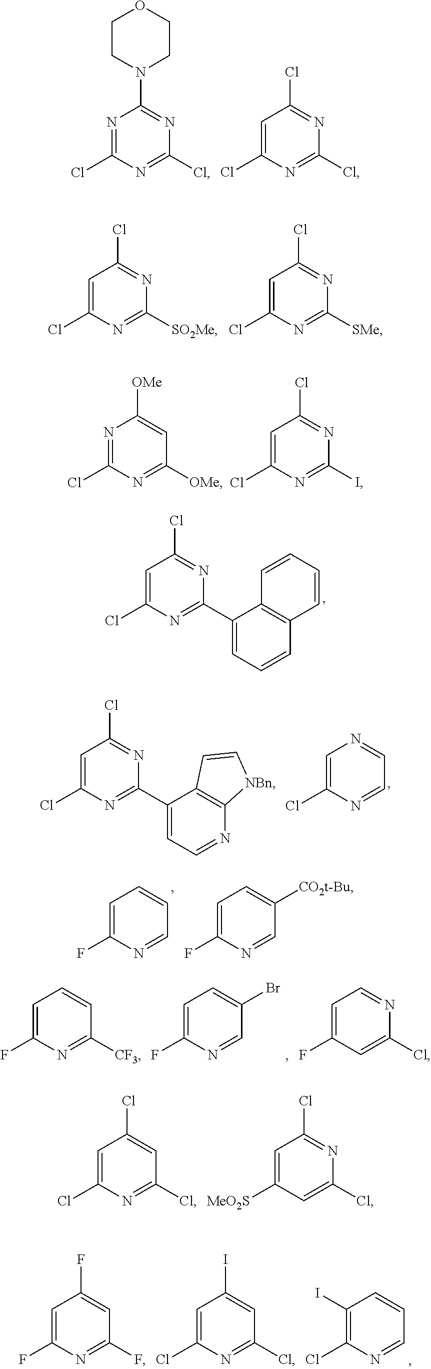

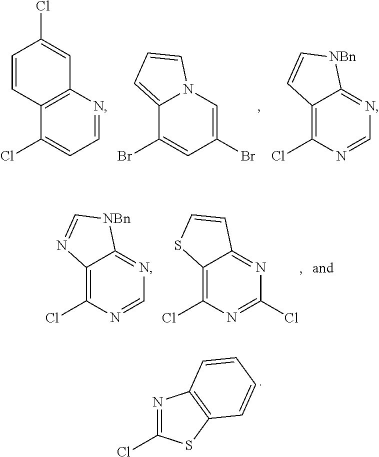

23. A compound of Formula I prepared according to the process of claim 1.
